(19) United States
(12) Patent Application Publication
(10) Pub. No.: US 2010/0063135 A1
(43) Pub. Date: Mar. 11, 2010

(54) POLYETHYLENE GLYCOL LIPID CONJUGATES AND USES THEREOF

(75) Inventors: Prasad A. Dande, Evanston, IL (US); Todd M. Hansen, Grayslake, IL (US); Robert D. Hubbard, Lindenhurst, IL (US); William E. Kohlbrenner, Libertyville, IL (US); Leiming Li, Buffalo Grove, IL (US); Aparna V. Sarthy, Waukegan, IL (US); Yu Shen, Gurnee, IL (US); Lu Tian, Blue Bell, PA (US); Carol K. Wada, Evanston, IL (US); Xiaobin Zhao, Potomac, MD (US)

Correspondence Address: PAUL D. YASGER;ABBOTT LABORATORIES, 100 ABBOTT PARK ROAD, DEPT. 377/AP6A, ABBOTT PARK, IL, 60064-6008, US

(73) Assignee: ABBOTT LABORATORIES, Abbott Park, IL (US)

(21) Appl. No.: 12/556,886

(22) Filed: Sep. 10, 2009

Related U.S. Application Data
(60) Provisional application No. 61/095,769, filed on Sep. 10, 2008.
(60) Provisional application No. 61/098,577, filed on Sep. 19, 2008.
(60) Provisional application No. 61/099,263, filed on Sep. 23, 2008.
(60) Provisional application No. 61/169,986, filed on Apr. 16, 2009.
(60) Provisional application No. 61/170,023, filed on Apr. 16, 2009.
(60) Provisional application No. 61/170,015, filed on Apr. 16, 2009.

(51) Int. Cl. A61K 31/7105; C07C 229/00; C07F 9/02; C07C 233/01; C07H 21/02; C07K 17/00; A61K 31/56; A61K 31/225; A61K 31/40

(52) U.S. Cl. 514/44 R; 554/213; 554/103; 554/79; 564/201; 536/23.1; 530/402; 514/182; 514/547; 514/408

(57) ABSTRACT

Polyethylene glycol (PEG)-lipid conjugates, polyethylene glycol (PEG)-lipid conjugate based drug delivery systems, ways to make them and methods of treating diseases using them are disclosed.

This application claims priority to U.S. Provisional Application Ser. No. 61/095,769 filed Sep. 10, 2008, U.S. Provisional Application Ser. No. 61/098,577 filed Sep. 19, 2008, U.S. Provisional Application Ser. No. 61/099,263 filed Sep. 23, 2008, U.S. Provisional Application Ser. No. 61/169,986 filed Apr. 16, 2009, U.S. Provisional Application Ser. No. 61/170,023 filed Apr. 16, 2009, and U.S. Provisional Application Ser. No. 61/170,015 filed Apr. 16, 2009, which are incorporated by reference in their entirety.

### FIELD OF THE INVENTION

This invention pertains to polyethylene glycol (PEG)-lipid conjugates, polyethylene glycol (PEG)-lipid conjugate based drug delivery systems, ways to make them, and methods of treating diseases using them.

### BACKGROUND OF THE INVENTION

Through the development of novel delivery formulations, research is now able to focus more on improving efficacy on the therapeutic and clinical efficacious of therapeutic agents such as nucleic acids, RNA, antisense oligonucleotide, a DNA, a plasmid, a ribosomal RNA (rRNA), a micro RNA (miRNA), transfer RNA (tRNA), a small inhibitory RNA (siRNA), and small nuclear RNA (snRNA). Such novel delivery formulations will need, for example, to allow for appropriate internalization of the therapeutic agent into the cell, agents sufficient absorption from the site of administration, distribution to various tissues, sufficient residence time, concentration at the sites of action to elicit effective biologic response, while minimizing toxicity, in addition to also maintaining it's stability, and size. To this end, many efforts have been made to develop liposome or cationic polymer complexes with polyethylene glycol (PEG) or other neutral or targeting moieties. Ogris et al., Gene Ther. 6, 595-605 (1999).

However, many of the agents to date have not been found to successfully deliver therapeutic agents or to successfully deliver therapeutic agents while minimizing toxicity. As such, there is a clear need in the art to develop a novel delivery system with an improved toxicity profile as well as enhanced therapeutic agent efficacy.

### SUMMARY OF THE INVENTION

One embodiment of this invention pertains to polyethylene glycol (PEG)-lipid conjugates, or mixtures thereof, having Formula I

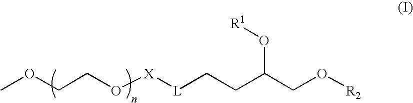

wherein
R¹ and R² are independently R³, or C(O)R³; or
R¹ and R² together are C(R³)₂;
R³ is C₈-C₂₄ alkyl;
L is C(OCH₃)₂, NHC(O), C(O)NH, OC(O)NH, NHC(O)O, NHC(O)NH, N(N)C(O), C(O)N(N), SS, NHC(O)L²C(O)O, NHC(O)L²C(O)NH, OC(O)L²C(O)O, OC(O)L²C(O)NH, C(O)O, OC(O), S, O, CH₂CH(═N)NHR⁴C(O), or C(═NNHCH₃)R⁴;
R⁴ is aryl or heteroaryl;
L² is C₁-C₆ alkyl;
X is a bond or C₁-C₆ alkyl; and
n is 10-200.

A further embodiment pertains to Cationic-Based Lipid Encapsulation Systems (CaBLES) comprising one or more non-cationic lipids, one or more polyethylene glycol (PEG)-lipid conjugates having Formula I and one or more cationic lipids.

In still a further embodiment, Lipid-Based Particles of the present invention are defined as CaBLES which further comprise one or more therapeutic agent(s). Such Lipid-Based Particles can be used to deliver any of a variety of therapeutic agent(s), preferably said therapeutic agent is a nucleic acid encoded with a product of interest, including but not limited to, RNA, antisense oligonucleotide, a DNA, a plasmid, a ribosomal RNA (rRNA), a micro RNA (miRNA), transfer RNA (tRNA), a small inhibitory RNA (siRNA), small nuclear RNA (snRNA), antigens, fragments thereof, proteins, peptides, vaccines and small-molecules or mixtures thereof.

A further embodiment pertains to pharmaceutical compositions comprising a Lipid-Based Particle and a pharmaceutically acceptable carrier.

A further embodiment pertains to a method of treating cancer in a mammal comprising administering thereto a therapeutically acceptable amount of a Lipid-Based Particle. Yet another embodiment pertains to a method of decreasing tumor volume in a mammal comprising administering thereto a therapeutically acceptable amount of a Lipid-Based Particle.

A further embodiment pertains to a method of making CaBLES or Lipid-Based Particles, comprising: (a) mixing the cationic lipid(s), the non-cationic lipid(s) and the PEG-lipid conjugate(s); (b) adding the mixture of step (a) to one or more therapeutic agents; and (c) separating and purifying resulting suspension of step (b).

### DESCRIPTION OF THE DRAWINGS

FIG. 1 illustrates in vivo activity of Lipid-Based Particle 1 (LPB1) and Lipid-Based Particle 2 (LPB2) versus a non-targeted composition (NTC).

FIG. 2 illustrates in vivo activity of Lipid-Based Particle 3 (LBP3) and Lipid-Based Particle 4 (LBP4) versus a non-targeted composition (NTC).

FIG. 3 illustrates in vivo activity of Lipid-Based Particles (LBP4-LBP12) versus a non-targeted composition (NTC).

FIG. 4 illustrates in vivo activity of a Lipid-Based Particle (LBP13) versus a non-targeted composition (NTC).

FIG. 5 illustrates in vivo activity of Lipid-Based Particles (LBP14, LBP15, and LBP16) versus a non-targeted composition (NTC).

FIG. 6 illustrates in vivo activity of Lipid-Based Particles (LBP17, LBP18) versus a non-targeted composition (NTC).

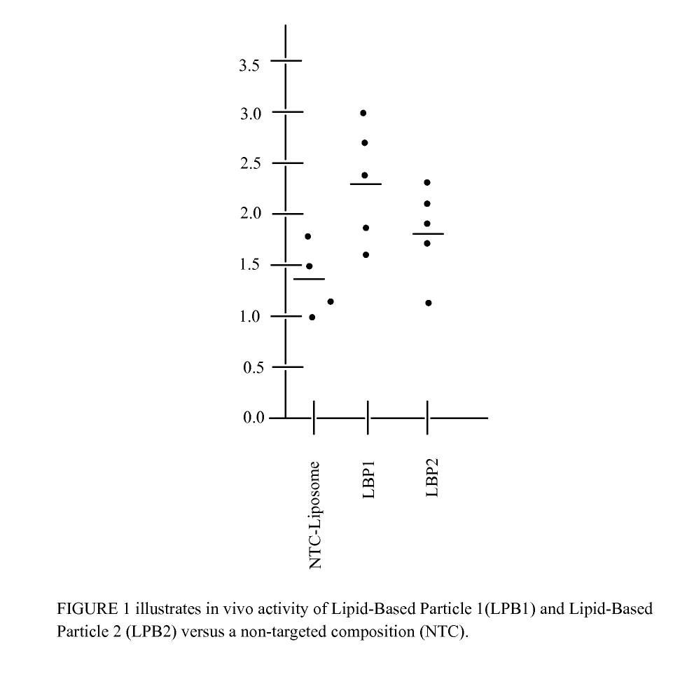

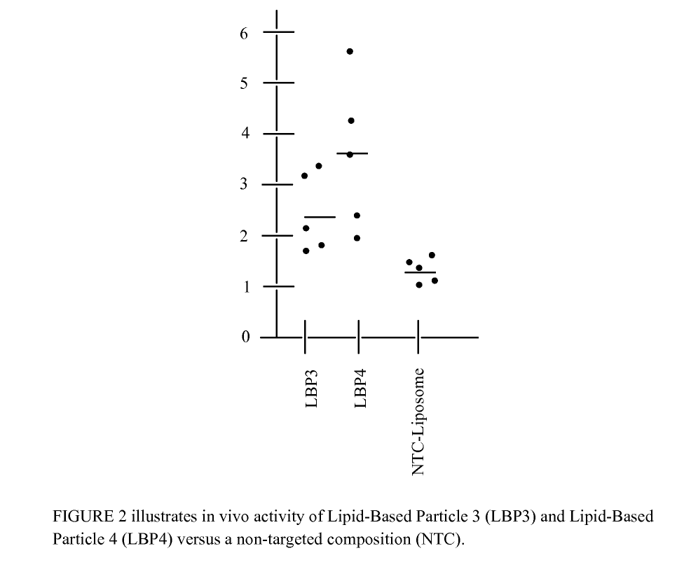

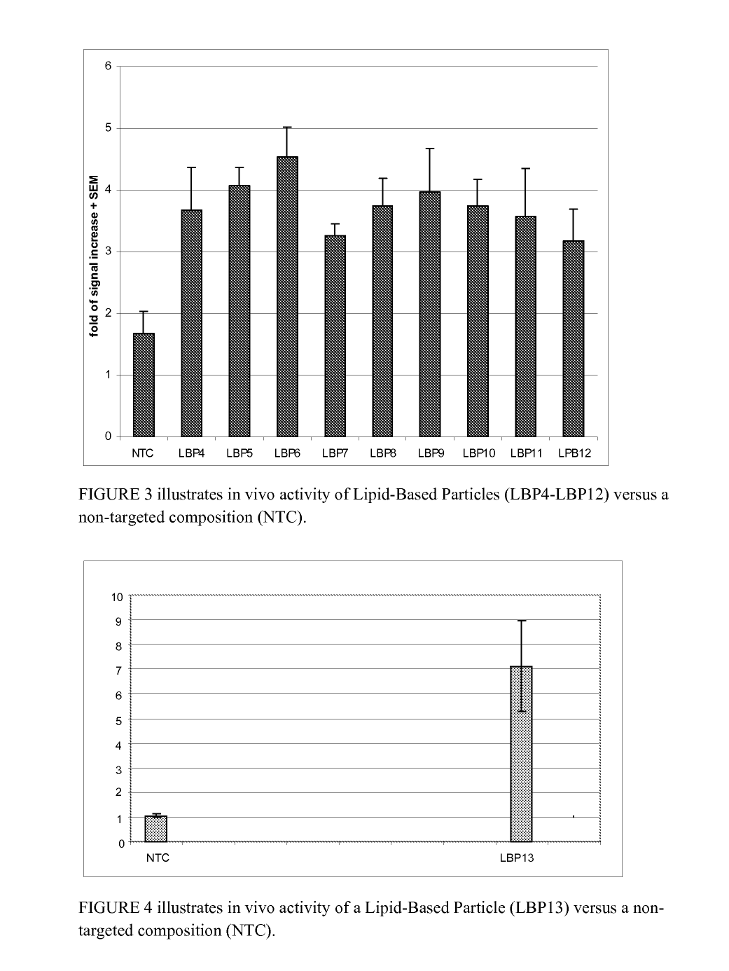

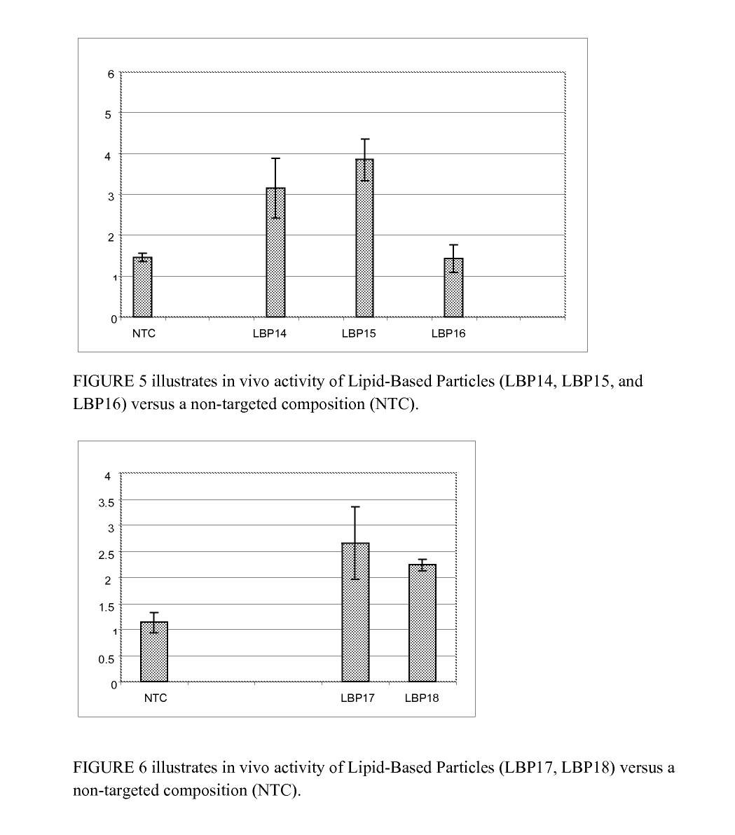

### DETAILED DESCRIPTION OF THE INVENTION

This invention pertains to in vitro and in vivo delivery of therapeutic agents. In particular, the invention pertains to compositions that allow for delivery of nucleic acids, including but not limited to RNA, antisense oligonucleotide, a DNA, a plasmid, a ribosomal RNA (rRNA), a micro RNA (miRNA), transfer RNA (tRNA), a small inhibitory RNA (siRNA), small nuclear RNA (snRNA), antigens, fragments thereof, proteins, peptides, and small molecules.

Variable moieties of compounds herein are represented by identifiers (capital letters with numerical and/or alphabetical superscripts) and may be specifically embodied.

It is also meant to be understood that a specific embodiment of a variable moiety may be the same or different as another specific embodiment having the same identifier and that asymmetric divalent moieties are drawn from left to right.

As used in the specification and the appended claims, unless specified to the contrary, the following terms have the meaning indicated:

The term “alkenyl,” as used herein, means monovalent, straight or branched chain hydrocarbon moieties having one or more than one carbon-carbon double bonds, such as C₂-alkenyl, C₃-alkenyl, C₄-alkenyl, C₅-alkenyl, C₆-alkenyl and the like.

The term “C₁-C₆-alkylene,” as used herein, means divalent, saturated, straight or branched chain hydrocarbon moieties bonds, such as C₁-alkylene, C₂-alkylene, C₃-alkylene, C₄-alkylene, C₅-alkylene, and C₆-alkylene.

The terms “alkyl,” as used herein, means monovalent, straight or branched chain hydrocarbon moieties such as C₁-alkyl, C₂-alkyl, C₃-alkyl, C₄-alkyl, C₅-alkyl, C₆-alkyl and the like.

The term “alkynyl,” as used herein, means monovalent, straight or branched chain hydrocarbon moieties having one or more than one carbon-carbon triple bonds, such as C₂-alkynyl, C₃-alkynyl, C₄-alkynyl, C₅-alkynyl, C₆-alkynyl and the like.

The term “C₁-C₆-alkyl” as used herein, means C₁-alkyl, C₂-alkyl, C₃-alkyl, C₄-alkyl, C₅-alkyl, and C₆-alkyl.

The term “C₈-C₂₄-alkenyl,” as used herein, means C₈-alkenyl, C₉-alkenyl, C₁₀-alkenyl, C₁₁-alkenyl, C₁₂-alkenyl, C₁₃-alkenyl, C₁₄-alkenyl, C₁₅-alkenyl, C₁₆-alkenyl, C₁₇-alkenyl, C₁₈-alkenyl, C₁₉-alkenyl, C₂₀-alkenyl C₂₁-alkenyl, C₂₂-alkenyl, C₂₃-alkenyl, and C₂₄-alkenyl.

The term “C₈-C₂₄-alkyl,” as used herein, means C₈-alkyl, C₉-alkyl, C₁₀-alkyl, C₁₁-alkyl, C₁₂-alkyl, C₁₃-alkyl, C₁₄-alkyl, C₁₅-alkyl, C₁₆-alkyl, C₁₇-alkyl, C₁₈-alkyl, C₁₉-alkyl, C₂₀-alkyl C₂₁-alkyl, C₂₂-alkyl, C₂₃-alkyl, and C₂₄-alkyl.

The term “aryl,” as used herein, means phenyl, a bicyclic aryl or a tricyclic aryl. The bicyclic aryl is naphthyl, a phenyl fused to a cycloalkyl, or a phenyl fused to a cycloalkenyl. The bicyclic aryl is attached to the parent molecular moiety through any carbon atom contained within the bicyclic aryl. Representative examples of the bicyclic aryl include, but are not limited to, dihydroindenyl, indenyl, naphthyl, dihydronaphthalenyl, and tetrahydronaphthalenyl. The tricyclic aryl is anthracene or phenanthrene, or a bicyclic aryl fused to a cycloalkyl, or a bicyclic aryl fused to a cycloalkenyl, or a bicyclic aryl fused to a phenyl. The tricyclic aryl is attached to the parent molecular moiety through any carbon atom contained within the tricyclic aryl. Representative examples of tricyclic aryl ring include, but are not limited to, azulenyl, dihydroanthracenyl, fluorenyl, and tetrahydrophenanthrenyl.

The term “cycloalkane,” as used herein, means saturated cyclic or bicyclic hydrocarbon moieties, such as C₃-cycloalkane, C₄-cycloalkane, C₅-cycloalkane, C₆-cycloalkane and the like.

The term “cycloalkyl,” as used herein, means monovalent, saturated cyclic and bicyclic hydrocarbon moieties, such as C₃-cycloalkyl, C₄-cycloalkyl, C₅-cycloalkyl, C₆-cycloalkyl and the like.

The term “cycloalkene,” as used herein, means cyclic and bicyclic hydrocarbon moieties having one or more than one carbon-carbon double bonds, such as C₅-cycloalkene, C₆-cycloalkene and the like.

The term “cycloalkenyl,” as used herein, means monovalent, cyclic hydrocarbon moieties having one or more than one carbon-carbon double bonds, such as C₄-cycloalkenyl, C₅-cycloalkenyl, C₆-cycloalkenyl and the like.

The term “heteroarene,” as used herein, means a five-membered or six-membered aromatic ring having at least one carbon atom and one or more than one independently selected nitrogen, oxygen or sulfur atom. The heteroarenes of this invention are connected through any adjacent atoms in the ring, provided that proper valences are maintained. Examples of heteroarenes include, but are not limited to furan, imidazole, isothiazole, isoxazole, oxadiazole, oxazole, pyrazine, pyrazole, pyridazine, pyridine, pyrimidine, pyrrole, thiazole, thiadiazole thiophene, tetrazine, tetrazole, triazine, triazole and the like.

The term “heteroaryl,” as used herein, means a monocyclic heteroaryl or a bicyclic heteroaryl. The monocyclic heteroaryl is a 5 or 6 membered ring. The 5 membered ring contains two double bonds and one, two, three or four nitrogen atoms and optionally one oxygen or sulfur atom. The 6 membered ring contains three double bonds and one, two, three or four nitrogen atoms. The 5 or 6 membered heteroaryl is connected to the parent molecular moiety through any carbon atom or any substitutable nitrogen atom contained within the heteroaryl, provided that proper valance is maintained. Representative examples of monocyclic heteroaryl include, but are not limited to, furyl, imidazolyl, isoxazolyl, isothiazolyl, oxadiazolyl, oxazolyl, pyridinyl, pyridazinyl, pyrimidinyl, pyrazinyl, pyrazolyl, pyrrolyl, tetrazolyl, thiadiazolyl, thiazolyl, thienyl, triazolyl, and triazinyl. The bicyclic heteroaryl consists of a monocyclic heteroaryl fused to a phenyl, or a monocyclic heteroaryl fused to a cycloalkyl, or a monocyclic heteroaryl fused to a cycloalkenyl, or a monocyclic heteroaryl fused to a monocyclic heteroaryl. The bicyclic heteroaryl is connected to the parent molecular moiety through any carbon atom or any substitutable nitrogen atom contained within the bicyclic heteroaryl, provided that proper valance is maintained. Representative examples of bicyclic heteroaryl include, but are not limited to, benzofuranyl, benzoxadiazolyl, benzoisoxazole, benzoisothiazole, benzooxazole, 1,3-benzothiazolyl, benzothiophenyl, cinnolinyl, furopyridine, indolyl, indazolyl, isobenzofuran, isoindolyl, isoquinolinyl, naphthyridinyl, oxazolopyridine, quinolinyl, quinoxalinyl and thienopyridinyl.

The term “heterocycloalkane,” as used herein, means cycloalkane having one or two or three CH₂ moieties replaced with independently selected O, S, S(O), SO₂ or NH and one or two CH moieties unreplaced or replaced with N and also means cycloalkane having one or two or three CH₂ moieties unreplaced or replaced with independently selected O, S, S(O), SO₂ or NH and one or two CH moieties replaced with N.

The term “heterocycloalkene,” as used herein, means cycloalkene having one or two or three CH₂ moieties replaced with independently selected O, S, S(O), SO₂ or NH and one or two CH moieties unreplaced or replaced with N and also means cycloalkene having one or two or three CH₂ moieties unreplaced or replaced with independently selected O, S, S(O), SO₂ or NH and one or two CH moieties replaced with N.

The term “heterocycloalkyl,” as used herein, means cycloalkyl having one or two or three CH₂ moieties replaced with independently selected O, S, S(O), SO₂ or NH and one or two CH moieties unreplaced or replaced with N and also means cycloalkyl having one or two or three CH₂ moieties unreplaced or replaced with independently selected O, S, S(O), SO₂ or NH and one or two CH moieties replaced with N.

The term “heterocycloalkenyl,” as used herein, means cycloalkenyl having one or two or three CH₂ moieties replaced with independently selected O, S, S(O), SO₂ or NH and one or two CH moieties unreplaced or replaced with N and also means cycloalkenyl having one or two or three CH₂ moieties unreplaced or replaced with independently selected O, S, S(O), SO₂ or NH and one or two CH moieties replaced with N.

The term “cyclic moiety,” as used herein, means benzene, cycloalkane, cycloalkyl, cycloalkene, cycloalkenyl, heteroarene, heteroaryl, heterocycloalkane, heterocycloalkyl, heterocycloalkene, heterocycloalkenyl and phenyl.

The term “DSPC,” as used herein, means 1,2-distearoyl-sn-glycero-3-phosphocholine.

The term, “Chol,” as used herein, means cholesterol.

The term, “PEG-Chol,” as used herein, means poly(oxy-1,2-ethanediyl)-2000-α-(3β)-cholest-5-en-3-yl-omega-hydroxy.

The term, “Pal-PEG-Cera,” as used herein, means N-palmitoyl-sphingosine-1-[succinyl(methoxypolyethylene glycol)-2000].

The term, “PEG-DMPE,” as used herein, means N-(carbonyl-methoxypolyethyleneglycol-2000)-1,2-dimyristoyl-sn-glycero-3-phosphoethanolamine.

The term, “PEG-DPPE,” as used herein, means N-(carbonyl-methoxypolyethyleneglycol-2000)-1,2-dipalmitoyl-sn-glycero-3-phosphoethanolamine.

The term, “PEG-DSPE,” as used herein, means N-(carbonyl-methoxypolyethyleneglycol-2000)-1,2-distearoyl-sn-glycero-3-phosphoethanolamine.

The term, “PEG-DMG,” as used herein, means 1,2-dimyristoyl-sn-glycerol-methoxypolyethyleneglycol-2000.

The term, “PEG-DPG,” as used herein, means 1,2-dipalmitoyl-sn-glycerol-methoxypolyethyleneglycol-2000.

The term, “PEG-DSG,” as used herein, means 1,2-distearoyl-sn-glycerol-methoxypolyethyleneglycol-2000.

The term “MALDI,” as used herein, means matrix assisted laser desorption ionization.

The term, “particle,” as used herein, means a small object that behaves as a whole unit in terms of its transport and properties.

The term, “nanoparticle,” as used herein, means any particle having a diameter of less than 1000 nanometers. In some embodiments, nanoparticles have a diameter of 500 or less. In some embodiments, nanoparticles have a diameter of 200 or less.

The term “nucleic acid” or “polynucleotide” refers to a polymer containing at least two deoxyribonucleotides or ribonucleotides in either single- or double-stranded form. Nucleic acids include nucleic acids containing known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphorothioates, phosphoramidates, methyl phosphonates, chiral-methyl phosphonates, 2-O-methyl ribonucleotides, peptide-nucleic acids (PNAs). Unless specifically limited, the terms encompasses nucleic acids containing known analogues of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al., Nucleic Acid Res. 19:5081 (1991); Ohtsuka et al., J. Biol. Chem. 260:2605-2608 (1985); and Cassol et al. (1992); Rossolini et al., Mol. Cell. Probes 8:91-98 (1994)). “Nucleotides” contain a sugar deoxyribose (DNA) or ribose (RNA), a base, and a phosphate group. Nucleotides are linked together through the phosphate groups. Nucleotides include chemically modified nucleotides as described in, e.g., WO 03/74654. “Bases” include purines and pyrimidines, which further include natural compounds adenine, thymine, guanine, cytosine, uracil, inosine, and natural analogs, and synthetic derivatives of purines and pyrimidines, which include, but are not limited to, modifications which place new reactive groups such as, but not limited to, amines, alcohols, thiols, carboxylates, and alkylhalides. DNA may be in the form of antisense, plasmid DNA, parts of a plasmid DNA, pre-condensed DNA, product of a polymerase chain reaction (PCR), vectors (P1, PAC, BAC, YAC, artificial chromosomes), expression cassettes, chimeric sequences, chromosomal DNA, or derivatives of these groups. The term nucleic acid is used interchangeably with gene, plasmid, cDNA, mRNA, and an interfering RNA molecule (e.g. a synthesized siRNA or an siRNA expressed from a plasmid).

The term, “siRNA,” as used herein, means a small inhibitory RNA, and molecules having endogenous RNA bases or chemically modified nucleotides. The modifications shall not abolish cellular activity, but rather impart increased stability and/or increased cellular potency. Examples of chemical modifications include phosphorothioate groups, 2′-deoxynucleotide, 2′-OCH₃-containing ribonucleotides, 2′-F-ribonucleotides, 2′-methoxyethyl ribonucleotides or a combination thereof.

The term, “SPC,” as used herein, means soybean phosphatidylcholine.

The term “small molecule,” as used herein, means antibiotics, antineoplastics, antiinflammatories, antivirals, immunomodulators and agents that act upon the respiratory system, the cardiovascular system, the central nervous system or a metabolic pathway involved with dyslipidemia, diabetes or Syndrome X.

The term, “NTC,” as used herein, means a non-targeted composition containing one or more (PEG)-lipid conjugates, one or more non-cationic lipids, one or more cationic lipids, and one or more non-targeted agents such as a non-targeted siRNA (sequence: UGGUUUACAUGUUGUGUGA SEQ ID NO: 1).

### Compounds

Compounds of this invention may contain asymmetrically substituted carbon atoms in the R or S configuration, wherein the terms “R” and “S” are as defined in Pure Appl. Chem. (1976) 45, 13-10. Compounds having asymmetrically substituted carbon atoms with equal amounts of R and S configurations are racemic at those atoms. Atoms having excess of one configuration over the other are assigned the configuration in excess, preferably an excess of about 85%-90%, more preferably an excess of about 95%-99%, and still more preferably an excess greater than about 99%. Accordingly, this invention is meant to embrace racemic mixtures and relative and absolute diastereoisomers and the compounds thereof.

Compounds of this invention may also contain carbon-carbon double bonds or carbon-nitrogen double bonds in the E or Z configuration, wherein the term “E” represents higher order substituents on opposite sides of the carbon-carbon or carbon-nitrogen double bond and the term “Z” represents higher order substituents on the same side of the carbon-carbon or carbon-nitrogen double bond as determined by the Cahn-Ingold-Prelog Priority Rules. The compounds of this invention may also exist as a mixture of “E” and “Z” isomers.

Compounds of this invention can exist in an isotopic form containing one or more atoms having an atomic mass or mass number different from the atomic mass or mass number most abundantly found in nature. Isotopes of atoms such as hydrogen, carbon, phosphorous, sulfur fluorine, chlorine, and iodine include, but are not limited to, ²H, ³H, ¹⁴C, ³²P, ³⁵S, ¹⁸F, ³⁶Cl, and ¹²⁵I, respectively. Compounds that contain other isotopes of these and/or other atoms are within the scope of this invention. Compounds containing tritium (³H) and ¹⁴C radioisotopes are preferred in general for their ease in preparation and detectability for radiolabeled compounds. Isotopically labeled compounds of this invention can be prepared by the general methods well known to persons having ordinary skill in the art. Such isotopically labeled compounds can be conveniently prepared by carrying out the procedures disclosed in the Examples and Schemes herein by substituting a readily available isotopically labeled reagent for a non-isotopically labeled reagent.

Suitable groups for X, L, R¹, R², R³, R⁴, L², and n in compounds of Formula (I) are independently selected. The described embodiments of the present invention may be combined. Such combination is contemplated and within the scope of the present invention. For example, it is contemplated that embodiments for any of X, L, R¹, R², R³, R⁴, L², and n can be combined with embodiments defined for any other of X, L, R¹, R², R³, R⁴, L², and n.

One embodiment of this invention pertains to polyethylene glycol (PEG)-lipid conjugates, or mixtures thereof, having Formula I

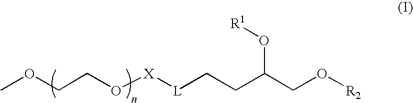

wherein
R¹ and R² are independently R³, or C(O)R³; or
R¹ and R² together are C(R³)₂;
R³ is C₈-C₂₄ alkyl;
L is C(OCH₃)₂, NHC(O), C(O)NH, OC(O)NH, NHC(O)O, NHC(O)NH, N(N)C(O), C(O)N(N), SS, NHC(O)L²C(O)O, NHC(O)L²C(O)NH, OC(O)L²C(O)O, OC(O)L²C(O)NH, C(O)O, OC(O), S, O, CH₂CH(═N)NHR⁴C(O), or C(═NNHCH₃)R⁴;
R⁴ is aryl or heteroaryl;
L² is C₁-C₆ alkyl;
X is a bond or C₁-C₆ alkyl; and
n is 10-200.

Another embodiment of this invention pertains to polyethylene glycol (PEG)-lipid conjugates, or mixtures thereof, having Formula I

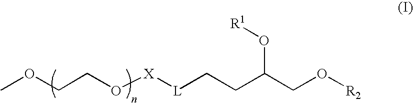

wherein
R¹ and R² are independently R³, or C(O)R³;
R³ is C₈-C₂₄ alkyl;

### L is C(O)NH, OC(O)NH, NHC(O)L²C(O)NH, or OC(O)L²C(O)NH;

L² is C₁-C₆ alkyl;
X is a bond or C₁-C₆ alkyl; and
n is 10-200.

In one embodiment of Formula I, R¹ and R² are independently R³. In another embodiment of Formula I, R¹ and R² are independently C(O)R³. In another embodiment of Formula I, one of R¹ and R² is R³, and the other is C(O)R³.

In one embodiment of Formula I, each R³ is independently C₁₀-C₁₈-alkyl. In another embodiment of Formula I, each R³ is C₁₀-alkyl. In another embodiment of Formula I, each R³ is C₁₁-alkyl. In another embodiment of Formula I, each R³ is C₁₂-alkyl. In another embodiment of Formula I, each R³ is C₁₃-alkyl. In another embodiment of Formula I, each R³ is C₁₄-alkyl. In another embodiment of Formula I, each R³ is C₁₅-alkyl. In another embodiment of Formula I, each R³ is C₁₆-alkyl. In another embodiment of Formula I, each R³ is C₁₇-alkyl. In another embodiment of Formula I, each R³ is C₁₈-alkyl. In another embodiment of Formula I, each R³ is decanyl-alkyl. In another embodiment of Formula I, each R³ is undecanyl-alkyl. In another embodiment of Formula I, each R³ is dodecanyl-alkyl. In another embodiment of Formula I, each R³ is tridecanyl-alkyl. In another embodiment of Formula I, each R³ is tetradecanyl-alkyl. In another embodiment of Formula I, each R³ is pentadecanyl-alkyl. In another embodiment of Formula I, each R³ is hexadecanyl-alkyl. In another embodiment of Formula I, each R³ is heptadecanyl-alkyl. In another embodiment of Formula I, each R³ is octadecanyl-alkyl.

In another embodiment of Formula I, one R³ is C₁₃-alkyl, and the other is C₁₄-alkyl. In another embodiment of Formula I, one R³ is C₁₃-alkyl, and the other is C₁₅-alkyl. In another embodiment of Formula I, one R³ is C₁₃-alkyl, and the other is C₁₆-alkyl. In another embodiment of Formula I, one R³ is C₁₃-alkyl, and the other is C₁₇-alkyl. In another embodiment of Formula I, one R³ is C₁₃-alkyl, and the other is C₁₈-alkyl. In another embodiment of Formula I, one R³ is tridecanyl-alkyl, and the other is tetradecanyl. In another embodiment of Formula I, one R³ is tridecanyl-alkyl, and the other is pentadecanyl-alkyl. In another embodiment of Formula I, one R³ is tridecanyl-alkyl, and the other is hexadecanyl-alkyl. In another embodiment of Formula I, one R³ is tridecanyl-alkyl, and the other is heptadecanyl. In another embodiment of Formula I, one R³ is tridecanyl-alkyl, and the other is octadecanyl-alkyl.

In another embodiment of Formula I, one R³ is C₁₄-alkyl, and the other is C₁₅-alkyl. In another embodiment of Formula I, one R³ is C₁₄-alkyl, and the other is C₁₆-alkyl. In another embodiment of Formula I, one R³ is C₁₄-alkyl, and the other is C₁₇-alkyl. In another embodiment of Formula I, one R³ is C₁₄-alkyl, and the other is C₁₈-alkyl. In another embodiment of Formula I, one R³ is tridecanyl-alkyl, and the other is pentadecanyl-alkyl. In another embodiment of Formula I, one R³ is tetradecanyl-alkyl, and the other is hexadecanyl-alkyl. In another embodiment of Formula I, one R³ is tetradecanyl-alkyl, and the other is heptadecanyl. In another embodiment of Formula I, one R³ is tetradecanyl-alkyl, and the other is octadecanyl-alkyl.

In another embodiment of Formula I, one R³ is C₁₅-alkyl, and the other is C₁₆-alkyl. In another embodiment of Formula I, one R³ is C₁₅-alkyl, and the other is C₁₇-alkyl. In another embodiment of Formula I, one R³ is C₁₅-alkyl, and the other is C₁₈-alkyl. In another embodiment of Formula I, one R³ is pentadecanyl-alkyl, and the other is hexadecanyl-alkyl. In another embodiment of Formula I, one R³ is pentadecanyl-alkyl, and the other is heptadecanyl. In another embodiment of Formula I, one R³ is pentadecanyl-alkyl, and the other is octadecanyl-alkyl.

In another embodiment of Formula I, one R³ is C₁₆-alkyl, and the other is C₁₇-alkyl. In another embodiment of Formula I, one R³ is C₁₆-alkyl, and the other is C₁₈-alkyl. In another embodiment of Formula I, one R³ is hexadecanyl-alkyl, and the other is heptadecanyl. In another embodiment of Formula I, one R³ is hexadecanyl-alkyl, and the other is octadecanyl-alkyl.

In another embodiment of Formula I, one R³ is C₁₇-alkyl, and the other is C₁₈-alkyl. In another embodiment of Formula I, one R³ is heptadecanyl-alkyl, and the other is octadecanyl-alkyl.

In one embodiment of Formula I, X¹ is C₁-C₂-alkyl. In another embodiment of Formula I, X¹ is a bond. In another embodiment of Formula I, X¹ is C₁-alkyl. In another embodiment of Formula I, X¹ is C₂-alkyl.

In one embodiment of Formula I, L is C(O)NH, OC(O)NH, NHC(O)L²C(O)NH, or OC(O)L²C(O)NH. In another embodiment of Formula I, L is C(O)NH. In another embodiment of Formula I, L is OC(O)NH. In another embodiment of Formula I, L is NHC(O)L²C(O)NH. In another embodiment of Formula I, L is OC(O)L²C(O)NH.

In one embodiment of Formula I, L² is C₁-C₆ alkyl. In another embodiment of Formula I, L² is C₂ alkyl.

In one embodiment of Formula I, n is 20-120. In another embodiment of Formula I, n is 22. In another embodiment of Formula I, n is 45. In another embodiment of Formula I, n is 112.

In one embodiment of Formula I, R¹ and R² are independently R³, each R³ is C₁₃-alkyl, X¹ is C₁-alkyl, L is C(O)NH, and n is 45. In another embodiment of Formula I, R¹ and R² are independently R³, each R³ is C₁₄-alkyl, X¹ is C₁-alkyl, L is C(O)NH, and n is 45. In another embodiment of Formula I, R¹ and R² are independently R³, each R³ is C₁₆-alkyl, X¹ is C₁-alkyl, L is C(O)NH, and n is 45. In another embodiment of Formula I, R¹ and R² are independently R³, each R³ is C₁₈-alkyl, X¹ is C₁-alkyl, L is C(O)NH, and n is 45. In another embodiment of Formula I, R¹ and R² are independently R³, each R³ is C₁₄-alkyl, X¹ is a bond, L is C(O)NH, and n is 45. In another embodiment of Formula I, R¹ and R² are independently R³, each R³ is C₁₆-alkyl, X¹ is a bond, L is C(O)NH, and n is 45. In another embodiment of Formula I, R¹ and R² are independently R³, each R³ is C₁₈-alkyl, X¹ is a bond, L is C(O)NH, and n is 45. In another embodiment of Formula I, R¹ and R² are independently R³, each R³ is C₁₆-alkyl, X¹ is C₂-alkyl, L NHC(O)L²C(O)NH, L² is C₂-alkyl, and n is 45. In another embodiment of Formula I, R¹ and R² are independently C(O)R³, each R³ is C₁₃-alkyl, X¹ is C₂-alkyl, L is OC(O)NH, and n is 45. In another embodiment of Formula I, R¹ and R² are independently C(O)R³, each R³ is C₁₅-alkyl, X¹ is C₂-alkyl, L is OC(O)NH, and n is 45. In another embodiment of Formula I, R¹ and R² are independently R³, each R³ is C₁₆-alkyl, X¹ is C₂-alkyl, L is OC(O)L²C(O)NH, L² is C₂-alkyl, and n is 45. In another embodiment of Formula I, R¹ and R² are independently C(O)R³, each R³ is C₁₅-alkyl, X¹ is C₁-alkyl, L is C(O)NH, and n is 45. In another embodiment of Formula I, R¹ is C₁₈-alkyl, R² is C₁₀-alkyl, X¹ is C₁-alkyl, L is C(O)NH, and n is 45. In another embodiment of Formula I, R¹ and R² are independently R³, each R³ is C₁₀-alkyl, X¹ is C₁-alkyl, L is C(O)NH, and n is 45. In another embodiment of Formula I, R¹ is C₁₈-alkyl, R² is C₁₄-alkyl, X¹ is C₁-alkyl, L is C(O)NH, and n is 45. In another embodiment of Formula I, R¹ is C₁₈-alkyl, R² is C₁₆-alkyl, X¹ is C₁-alkyl, L is C(O)NH, and n is 45. In another embodiment of Formula I, R¹ and R² are independently R³, each R³ is C₁₆-alkyl, X¹ is C₁-alkyl, L is C(O)NH, and n is 22. In another embodiment of Formula I, R¹ and R² are independently R³, each R³ is C₁₆-alkyl, X¹ is C₁-alkyl, L is C(O)NH, and n is 112. In another embodiment of Formula I, R¹ is C₁₆-alkyl, R² is C₁₈-alkyl, X¹ is C₁-alkyl, L is C(O)NH, and n is 45.

Still another embodiment pertains to compounds of Formula I which are
6-oxo-2-(tetradecanoyloxy)-8,11,14,17,20,23,26,29,32,35,38,41,44,47,50,53,56,59,62,65,68,71,74,77,80,83,86,89,92, 95,98,101,104,107,110,113,116,119,122,125,128,131,134,137,140,143-hexatetracontaoxa-5-azatetratetracontahect-1-yl myristate;
N-[3,4-bis(tetradecyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide;
N-[3,4-bis(hexadecyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide;
N-[3,4-bis(octadecyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide;
3,7,11,15,19,23,27,31,35,39,43,47,51,55,59,63,67,71,75,79,83,87,91,95,99,103,107,111,115, 119,123,127,131,135,139,143,147,151,155,159,163,167,171,175,179,182-hexatetracontaoxatrioctacontahect-1-yl 3,4-bis(tetradecyloxy)butylcarbamate;
3,6,9,12,15,18,21,24,27,30,33,36,39,42,45,48,51,54,57,60,63,66,69,72,75,78,81,84,87,90, 93,96,99,102,105,108,111,114,117,120,123,126,129,132,135,138-hexatetracontaoxanonatriacontahect-1-yl 3,4-bis(hexadecyloxy)butylcarbamate;
3,6,9,12,15,18,21,24,27,30,33,36,39,42,45,48,51,54,57,60,63,66,69,72,75,78,81,84,87,90, 93,96,99,102,105,108,111,114,117,120,123,126,129,132,135,138-hexatetracontaoxanonatriacontahect-1-yl 3,4-bis(octadecyloxy)butylcarbamate;
N-[3,4-bis(hexadecyloxy)butyl]-N′-3,6,9,12,15,18,21,24,27,30,33,36,39,42,45,48,51,54,57,60,63,66,69,72,75,78,81,84,87,90, 93,96,99,102,105,108,111,114,117,120,123,126,129,132,135,138-hexatetracontaoxanonatriacontahect-1-ylsuccinamide;
6-oxo-2-(tetradecanoyloxy)-7,10,13,16,19,22,25,28,31,34,37,40,43,46,49,52,55,58,61,64,67,70,73,76,79,82,85,88,91, 94,97,100,103,106,109,112,115,118,121,124,127,130,133,136,139,142,145-heptatetracontaoxa-5-azahexatetracontahect-1-yl myristate;
6-oxo-2-(palmitoyloxy)-7,10,13,16,19,22,25,28,31,34,37,40,43,46,49,52,55,58,61,64,67,70,73,76,79,82,85,88,91, 94,97,100,103,106,109,112,115,118,121,124,127,130,133,136,139,142,145-heptatetracontaoxa-5-azahexatetracontahect-1-yl palmitate;
3,6,9,12,15,18,21,24,27,30,33,36,39,42,45,48,51,54,57,60,63,66,69,72,75,78,81,84,87,90, 93,96,99,102,105,108,111,114,117,120,123,126,129,132,135,138-hexatetracontaoxanonatriacontahect-1-yl 4-{[3,4-bis(hexadecyloxy)butyl]amino}-4-oxobutanoate;
6-oxo-2-(palmitoyloxy)-8,11,14,17,20,23,26,29,32,35,38,41,44,47,50,53,56,59,62,65,68,71,74,77,80,83,86,89,92, 95,98,101,104,107,110,113,116,119,122,125,128,131,134,137,140,143-hexatetracontaoxa-5-azatetratetracontahect-1-yl palmitate;
N-[4-(decyloxy)-3-(octadecyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide;
N-[3,4-bis(decyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide;
N-[3-(octadecyloxy)-4-(tetradecyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide;
N-[4-(hexadecyloxy)-3-(octadecyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide;
N-[3,4-bis(hexadecyloxy)butyl]-2,5,8,11,14,17,20,23,26,29,32,35,38,41,44,47,50,53,56,59,62,65,68-tricosaoxaheptacontan-70-amide;
N-[3,4-bis(hexadecyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137,140,143,146,149,152,155, 158,161,164,167,170,173,176,179,182,185,188,191,194,197,200,203,206,209,212,215, 218,221,224,227,230,233,236,239,242,245,248,251,254,257,260,263,266,269,272,275,278, 281,284,287,290,293,296,299,302,305,308,311,314,317,320,323,326,329,332,335,338-113oxa340n-340-amide; and
N-[3-(hexadecyloxy)-4-(octadecyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide.

### (PEG)-Lipid Conjugate-Based Lipid Encapsulation Systems, and Lipid-Based Particles

A still further embodiment pertains to Cationic-Based Lipid Encapsulation Systems (CaBLES) comprising non-cationic lipid(s), polyethylene glycol (PEG)-lipid conjugate(s) having Formula I and cationic lipid(s).

A still further embodiment pertains to Cationic-Based Lipid Encapsulation Systems (CaBLES) comprising one or more (PEG)-lipid conjugates having Formula (I)

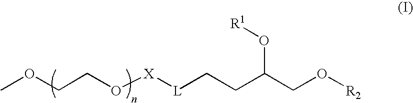

wherein
R¹ and R² are independently R³, or C(O)R³; or
R¹ and R² together are C(R³)₂;
R³ is C₁₂-C₂₄ alkyl;
L is C(OCH₃)₂, NHC(O), C(O)NH, OC(O)NH, NHC(O)O, NHC(O)NH, N(N)C(O), C(O)N(N), SS, NHC(O)L²C(O)O, NHC(O)L²C(O)NH, OC(O)L²C(O)O, OC(O)L²C(O)NH, C(O)O, OC(O), S, O, CH₂CH(═N)NHR⁴C(O), or C(═NNHCH₃)R⁴;
R⁴ is aryl or heteroaryl;
L² is C₁-C₆ alkyl;
X is a bond or C₁-C₆ alkyl; and
n is 10-200; and
one or more non-cationic lipids, and one or more cationic lipids.

In still a further embodiment, Lipid-Based Particles of the present invention are defined as CaBLES which further comprise one or more therapeutic agent(s). Therapeutic agents that can be delivered with CaBLES include RNA, antisense oligonucleotide, a DNA, a plasmid, a ribosomal RNA (rRNA), a micro RNA (miRNA), transfer RNA (tRNA), a small inhibitory RNA (siRNA), small nuclear RNA (snRNA), chimeric nucleic acids, an antigen, fragments thereof, a protein, a peptide, small-molecules, or mixtures thereof. This invention describes delivery of RNA's such as small inhibitory RNA or microRNA. The nucleic acid can have varying lengths (10-200 bps) and structures (hairpins, single/double strands, bulges, nicks/gaps, mismatches) and processed in the cell to provide active gene silencing. In certain embodiments of this invention, a double-stranded siRNA (dsRNA) can have the same number of nucleotides on each strand (blunt ends) or asymmetric ends (overhangs). The overhang of 1-2 nucleotides can be present on the sense and/or the antisense strand, as well as present on the 5′- and/or the 3′-ends of a given strand.

In one embodiment, the therapeutic agent is RNA, antisense oligonucleotide, a DNA, a plasmid, a ribozymal RNA (rRNA), a micro RNA (miRNA), transfer RNA (tRNA), a small inhibitory RNA (siRNA), small nuclear RNA (snRNA), an antigen, fragments thereof, a protein, a peptide, a small-molecule, or a mixture thereof.

In certain embodiments, the PEG lipid conjugate of the Lipid-Based Particle can have a ligand attached, such as a targeting ligand or a chelating moiety. Suitable targeting ligands include, but are not limited to, a compound or device with a reactive functional group and include lipids, amphipathic lipids, carrier compounds, bioaffinity compounds, biomaterials, biopolymers, biomedical devices, analytically detectable compounds, therapeutically active compounds, enzymes, peptides, proteins, antibodies, immune stimulators, radiolabels, fluorogens, biotin, drugs, haptens, DNA, RNA, polysaccharides, liposomes, virosomes, micelles, immunoglobulins, functional groups, other targeting moieties, or toxins.

In another embodiment, a targeting ligand (moiety) is conjugated to the periphery of the PEG-lipid in a Lipid-Based Particle formulation. Preferably, the targeting moiety is a ligand of a receptor present on a target cell and the receptor is preferentially expressed by the target cell versus a non-target cell. In one aspect, the targeting moiety is an antibody or fragments thereof. In one aspect, the targeting moiety is a small protein, or peptide. In another aspect, the targeting moiety is a small-molecule.

In still a further embodiment, these Lipid-Based Particles are nanoparticles and have mean diameter sizes of about 50-300 nm, of which 50-250 nm is preferred and 50-200 nm is most preferred.

A further embodiment pertains to CaBLES or Lipid-Base Particles wherein the PEG lipid conjugate(s) are about 0.1-20 weight/weight % of total lipid in particle, the non-cationic lipid(s) are about 1-30 weight/weight % of total lipid in particle, the cholesterol is about 5-45 weight/weight % of total lipid in particle, and the cationic lipid(s) are about 5-60 weight/weight % of total lipid in particle.

A further embodiment pertains to CaBLES or Lipid-Base Particles wherein the PEG lipid conjugate(s) are about 0.1-20 weight/weight % of total lipid in particle, the DSPC is about 1-30 weight/weight % of total lipid in particle, the cholesterol is about 5-45 weight/weight % of total lipid in particle, and the cationic lipid(s) are about 5-60 weight/weight % of total lipid in particle.

A further embodiment pertains to a pharmaceutical composition comprising a Lipid-Based Particle and a pharmaceutically acceptable carrier.

A further embodiment pertains to a pharmaceutical composition, wherein a Lipid-Based Particle comprises cholesterol, DSPC, 1-(2,3-bis((9Z,12Z)-octadeca-9,12-dienyloxy)propyl)pyrrolidine, N-[3,4-bis(tetradecyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide and one or more nucleic acids.

A further embodiment pertains to a pharmaceutical composition, wherein N-[3,4-bis(tetradecyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanon atriacontahectan-139-amide is about 1-25 weight/weight % of total lipid in particle, DSPC is about 1-30 weight/weight % of total lipid in particle, cholesterol is about 5-45 weight/weight % of total lipid in particle, and 1-(2,3-bis((9Z,12Z)-octadeca-9,12-dienyloxy)propyl)pyrrolidine is about 5-60 weight/weight % of total lipid in particle.

A further embodiment pertains to a Lipid-Based Particle, wherein the non-cationic lipids are cholesterol and DSPC, the cationic lipid is 1-(2,3-bis((9Z,12Z)-octadeca-9,12-dienyloxy)propyl)pyrrolidine, the PEG-lipid conjugate is N-[3,4-bis(tetradecyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanon atriacontahectan-139-amide, and the therapeutic agent is siRNA.

A further embodiment pertains to a Lipid-Based Particle, wherein the N-[3,4-bis(tetradecyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanon atriacontahectan-139-amide is about 1-25 weight/weight % of total lipid in particle, the DSPC is about 1-30 weight/weight % of total lipid in particle, the cholesterol is about 5-45 weight/weight % of total lipid in particle, and the 1-(2,3-bis((9Z,12Z)-octadeca-9,12-dienyloxy)propyl)pyrrolidine is about 5-60 weight/weight % of total lipid in particle.

A further embodiment pertains to a pharmaceutical composition, wherein the Lipid-Based Particle comprises cholesterol, DSPC, 1-(2,3-bis((9Z,12Z)-octadeca-9,12-dienyloxy)propyl)pyrrolidine, 2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide, 1,2-distearoyl-sn-glycerol-methoxypolyethyleneglycol-2000, and one or more nucleic acids.

A further embodiment pertains to a pharmaceutical composition, wherein the 2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide and 1,2-distearoyl-sn-glycerol-methoxypolyethyleneglycol-2000 are about 1-25 weight/weight % of total lipid in particle, the DSPC is about 1-30 weight/weight % of total lipid in particle, the cholesterol is about 5-45 weight/weight % of total lipid in particle, and 1-(2,3-bis((9Z,12Z)-octadeca-9,12-dienyloxy)propyl)pyrrolidine is about 5-60 weight/weight % of total lipid in particle.

A further embodiment pertains to a Lipid-Based Particle, wherein the non-cationic lipids are cholesterol and DSPC, the cationic lipid is 1-(2,3-bis((9Z,12Z)-octadeca-9,12-dienyloxy)propyl)pyrrolidine, the PEG-lipid conjugates are 2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide and 1,2-distearoyl-sn-glycerol-methoxypolyethyleneglycol-2000, and the therapeutic agent is siRNA.

A further embodiment pertains to a Lipid-Based Particle, wherein the 2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide and 1,2-distearoyl-sn-glycerol-methoxypolyethyleneglycol-2000 are about 1-25 weight/weight % of total lipid in particle, the DSPC is about 1-30 weight/weight % of total lipid in particle, the cholesterol is about 5-45 weight/weight % of total lipid in particle, and the 1-(2,3-bis((9Z,12Z)-octadeca-9,12-dienyloxy)propyl)pyrrolidine is about 5-60 weight/weight % of total lipid in particle.

A further embodiment pertains to a pharmaceutical composition, wherein the Lipid-Based Particle comprises cholesterol, DSPC, 1-(2,3-bis((9Z,12Z)-octadeca-9,12-dienyloxy)propyl)pyrrolidine, 2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide, 1,2-distearoyl-sn-glycerol-methoxypolyethyleneglycol-2000, and one or more nucleic acids.

A further embodiment pertains to a pharmaceutical composition, wherein the 2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide and 1,2-distearoyl-sn-glycerol-methoxypolyethyleneglycol-2000 are about 1-25 weight/weight % of total lipid in particle, the DSPC is about 1-30 weight/weight % of total lipid in particle, the cholesterol is about 5-45 weight/weight % of total lipid in particle, and 1-(2,3-bis((9Z,12Z)-octadeca-9,12-dienyloxy)propyl)pyrrolidine is about 5-60 weight/weight % of total lipid in particle.

A further embodiment pertains to a Lipid-Based Particle, wherein the non-cationic lipids are cholesterol and DSPC, the cationic lipid is 1-(2,3-bis((9Z,12Z)-octadeca-9,12-dienyloxy)propyl)pyrrolidine, the PEG-lipid conjugates are 2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide and 1,2-distearoyl-sn-glycerol-methoxypolyethyleneglycol-2000, and the therapeutic agent is siRNA.

A further embodiment pertains to a Lipid-Based Particle, wherein the 2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide and 1,2-distearoyl-sn-glycerol-methoxypolyethyleneglycol-2000 are about 1-25 weight/weight % of total lipid in particle, the DSPC is about 1-30 weight/weight % of total lipid in particle, the cholesterol is about 5-45 weight/weight % of total lipid in particle, and the 1-(2,3-bis((9Z,12Z)-octadeca-9,12-dienyloxy)propyl)pyrrolidine is about 5-60 weight/weight % of total lipid in particle.

A further embodiment pertains to a pharmaceutical composition, wherein the Lipid-Based Particle comprises cholesterol, DSPC, 1-(2,3-bis((9Z,12Z)-octadeca-9,12-dienyloxy)propyl)pyrrolidine, 2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide, N-(carbonyl-methoxypolyethyleneglycol-2000)-1,2-distearoyl-sn-glycero-3-phosphoethanolamine, and one or more nucleic acids.

A further embodiment pertains to a pharmaceutical composition, wherein the 2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide and N-(carbonyl-methoxypolyethyleneglycol-2000)-1,2-distearoyl-sn-glycero-3-phosphoethanolamine are about 1-25 weight/weight % of total lipid in particle, the DSPC is about 1-30 weight/weight % of total lipid in particle, the cholesterol is about 5-45 weight/weight % of total lipid in particle, and 1-(2,3-bis((9Z,12Z)-octadeca-9,12-dienyloxy)propyl)pyrrolidine is about 5-60 weight/weight % of total lipid in particle.

A further embodiment pertains to a Lipid-Based Particle, wherein the non-cationic lipids are cholesterol and DSPC, the cationic lipid is 1-(2,3-bis((9Z,12Z)-octadeca-9,12-dienyloxy)propyl)pyrrolidine, the PEG-lipid conjugates are 2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide and N-(carbonyl-methoxypolyethyleneglycol-2000)-1,2-distearoyl-sn-glycero-3-phosphoethanolamine, and the therapeutic agent is siRNA.

A further embodiment pertains to a Lipid-Based Particle, wherein the 2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide and N-(carbonyl-methoxypolyethyleneglycol-2000)-1,2-distearoyl-sn-glycero-3-phosphoethanolamine are about 1-25 weight/weight % of total lipid in particle, the DSPC is about 1-30 weight/weight % of total lipid in particle, the cholesterol is about 5-45 weight/weight % of total lipid in particle, and the 1-(2,3-bis((9Z,12Z)-octadeca-9,12-dienyloxy)propyl)pyrrolidine is about 5-60 weight/weight % of total lipid in particle.

A further embodiment pertains to a pharmaceutical composition, wherein the Lipid-Based Particle comprises cholesterol, DSPC, 1-{3,4-bis[(9Z,12Z)-octadeca-9,12-dienyloxy]butyl}pyrrolidine, 2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide and one or more nucleic acids.

A further embodiment pertains to a pharmaceutical composition, wherein the 2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide is about 1-25 weight/weight % of total lipid in particle, the DSPC is about 1-30 weight/weight % of total lipid in particle, the cholesterol is about 5-45 weight/weight % of total lipid in particle, and 1-{3,4-bis[(9Z,12Z)-octadeca-9,12-dienyloxy]butyl}pyrrolidine is about 5-60 weight/weight % of total lipid in particle.

A further embodiment pertains to a Lipid-Based Particle, wherein the non-cationic lipids are cholesterol and DSPC, the cationic lipid is 1-{3,4-bis[(9Z,12Z)-octadeca-9,12-dienyloxy]butyl}pyrrolidine, the PEG-lipid conjugate is 2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide, and the therapeutic agent is siRNA.

A further embodiment pertains to a Lipid-Based Particle, wherein the 2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide is about 1-25 weight/weight % of total lipid in particle, the DSPC is about 1-30 weight/weight % of total lipid in particle, the cholesterol is about 5-45 weight/weight % of total lipid in particle, and the 1-{3,4-bis[(9Z,12Z)-octadeca-9,12-dienyloxy]butyl}pyrrolidine is about 5-60 weight/weight % of total lipid in particle.

A further embodiment pertains to a pharmaceutical composition, wherein the Lipid-Based Particle comprises cholesterol, DSPC, 1-{3,4-bis[(9Z,12Z)-octadeca-9,12-dienyloxy]butyl}pyrrolidine, N-[3,4-bis(octadecyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide and one or more nucleic acids.

A further embodiment pertains to a pharmaceutical composition, wherein the N-[3,4-bis(octadecyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide is about 1-25 weight/weight % of total lipid in particle, the DSPC is about 1-30 weight/weight % of total lipid in particle, the cholesterol is about 5-45 weight/weight % of total lipid in particle, and 1-{3,4-bis[(9Z,12Z)-octadeca-9,12-dienyloxy]butyl}pyrrolidine is about 5-60 weight/weight % of total lipid in particle.

A further embodiment pertains to a Lipid-Based Particle, wherein the non-cationic lipids are cholesterol and DSPC, the cationic lipid is 1-{3,4-bis[(9Z,12Z)-octadeca-9,12-dienyloxy]butyl}pyrrolidine, the PEG-lipid conjugate is N-[3,4-bis(octadecyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide, and the therapeutic agent is siRNA.

A further embodiment pertains to a Lipid-Based Particle, wherein the N-[3,4-bis(octadecyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide is about 1-25 weight/weight % of total lipid in particle, the DSPC is about 1-30 weight/weight % of total lipid in particle, the cholesterol is about 5-45 weight/weight % of total lipid in particle, and the 1-{3,4-bis[(9Z,12Z)-octadeca-9,12-dienyloxy]butyl}pyrrolidine is about 5-60 weight/weight % of total lipid in particle.

A further embodiment pertains to a pharmaceutical composition, wherein the Lipid-Based Particle comprises cholesterol, DSPC, 1-(2,3-bis((9Z,12Z)-octadeca-9,12-dienyloxy)propyl)pyrrolidine, 6-oxo-2-(palmitoyloxy)-8,11,14,17,20,23,26,29,32,35,38,41,44,47,50,53,56,59,62,65,68,71,74,77,80,83,86,89,92, 95,98,101,104,107,110,113,116,119,122,125,128,131,134,137,140,143-hexatetracontaoxa-5-azatetratetracontahect-1-yl palmitate and one or more nucleic acids.

A further embodiment pertains to a pharmaceutical composition, wherein the 6-oxo-2-(palmitoyloxy)-8,11,14,17,20,23,26,29,32,35,38,41,44,47,50,53,56,59,62,65,68,71,74,77,80,83,86,89,92, 95,98,101,104,107,110,113,116,119,122,125,128,131,134,137,140,143-hexatetracontaoxa-5-azatetratetracontahect-1-yl palmitate is about 1-25 weight/weight % of total lipid in particle, the DSPC is about 1-30 weight/weight % of total lipid in particle, the cholesterol is about 5-45 weight/weight % of total lipid in particle, and 1-(2,3-bis((9Z,12Z)-octadeca-9,12-dienyloxy)propyl)pyrrolidine is about 5-60 weight/weight % of total lipid in particle.

A further embodiment pertains to a Lipid-Based Particle, wherein the non-cationic lipids are cholesterol and DSPC, the cationic lipid is 1-(2,3-bis((9Z,12Z)-octadeca-9,12-dienyloxy)propyl)pyrrolidine, the PEG-lipid conjugate is 6-oxo-2-(palmitoyloxy)-8,11,14,17,20,23,26,29,32,35,38,41,44,47,50,53,56,59,62,65,68,71,74,77,80,83,86,89,92, 95,98,101,104,107,110,113,116,119,122,125,128,131,134,137,140,143-hexatetracontaoxa-5-azatetratetracontahect-1-yl palmitate, and the therapeutic agent is siRNA.

A further embodiment pertains to a Lipid-Based Particle, wherein the 6-oxo-2-(palmitoyloxy)-8,11,14,17,20,23,26,29,32,35,38,41,44,47,50,53,56,59,62,65,68,71,74,77,80,83,86,89,92, 95,98,101,104,107,110,113,116,119,122,125,128,131,134,137,140,143-hexatetracontaoxa-5-azatetratetracontahect-1-yl palmitate is about 1-25 weight/weight % of total lipid in particle, the DSPC is about 1-30 weight/weight % of total lipid in particle, the cholesterol is about 5-45 weight/weight % of total lipid in particle, and the 1-(2,3-bis((9Z,12Z)-octadeca-9,12-dienyloxy)propyl)pyrrolidine is about 5-60 weight/weight % of total lipid in particle.

A further embodiment pertains to a pharmaceutical composition, wherein the Lipid-Based Particle comprises cholesterol, DSPC, 1-(2,3-bis((9Z,12Z)-octadeca-9,12-dienyloxy)propyl)pyrrolidine, N-[3,4-bis(hexadecyloxy)butyl]-N′-3,6,9,12,15,18,21,24,27,30,33,36,39,42,45,48,51,54,57,60,63,66,69,72,75,78,81,84,87,90, 93,96,99,102,105,108,111,114,117,120,123,126,129,132,135,138-hexatetracontaoxanonatriacontahect-1-ylsuccinamide and one or more nucleic acids.

A further embodiment pertains to a pharmaceutical composition, wherein the N-[3,4-bis(hexadecyloxy)butyl]-N′-3,6,9,12,15,18,21,24,27,30,33,36,39,42,45,48,51,54,57,60,63,66,69,72,75,78,81,84,87,90, 93,96,99,102,105,108,111,114,117,120,123,126,129,132,135,138-hexatetracontaoxanonatriacontahect-1-ylsuccinamide is about 1-25 weight/weight % of total lipid in particle, the DSPC is about 1-30 weight/weight % of total lipid in particle, the cholesterol is about 5-45 weight/weight % of total lipid in particle, and 1-(2,3-bis((9Z,12Z)-octadeca-9,12-dienyloxy)propyl)pyrrolidine is about 5-60 weight/weight % of total lipid in particle.

A further embodiment pertains to a Lipid-Based Particle, wherein the non-cationic lipids are cholesterol and DSPC, the cationic lipid is 1-(2,3-bis((9Z,12Z)-octadeca-9,12-dienyloxy)propyl)pyrrolidine, the PEG-lipid conjugate is N-[3,4-bis(hexadecyloxy)butyl]-N′-3,6,9,12,15,18,21,24,27,30,33,36,39,42,45,48,51,54,57,60,63,66,69,72,75,78,81,84,87,90, 93,96,99,102,105,108,111,114,117,120,123,126,129,132,135,138-hexatetracontaoxanonatriacontahect-1-ylsuccinamide, and the therapeutic agent is siRNA.

A further embodiment pertains to a Lipid-Based Particle, wherein the N-[3,4-bis(hexadecyloxy)butyl]-N′-3,6,9,12,15,18,21,24,27,30,33,36,39,42,45,48,51,54,57,60,63,66,69,72,75,78,81,84,87,90, 93,96,99,102,105,108,111,114,117,120,123,126,129,132,135,138-hexatetracontaoxanonatriacontahect-1-ylsuccinamide is about 1-25 weight/weight % of total lipid in particle, the DSPC is about 1-30 weight/weight % of total lipid in particle, the cholesterol is about 5-45 weight/weight % of total lipid in particle, and the 1-(2,3-bis((9Z,12Z)-octadeca-9,12-dienyloxy)propyl)pyrrolidine is about 5-60 weight/weight % of total lipid in particle.

A further embodiment pertains to a pharmaceutical composition, wherein the Lipid-Based Particle comprises cholesterol, DSPC, 1-(2,3-bis((9Z,12Z)-octadeca-9,12-dienyloxy)propyl)pyrrolidine, 3,6,9,12,15,18,21,24,27,30,33,36,39,42,45,48,51,54,57,60,63,66,69,72,75,78,81,84,87,90, 93,96,99,102,105,108,111,114,117,120,123,126,129,132,135,138-hexatetracontaoxanonatriacontahect-1-yl 4-{[3,4-bis(hexadecyloxy)butyl]amino}-4-oxobutanoate and one or more nucleic acids.

A further embodiment pertains to a pharmaceutical composition, wherein the 3,6,9,12,15,18,21,24,27,30,33,36,39,42,45,48,51,54,57,60,63,66,69,72,75,78,81,84,87,90, 93,96,99,102,105,108,111,114,117,120,123,126,129,132,135,138-hexatetracontaoxanonatriacontahect-1-yl 4-{[3,4-bis(hexadecyloxy)butyl]amino}-4-oxobutanoate is about 1-25 weight/weight % of total lipid in particle, the DSPC is about 1-30 weight/weight % of total lipid in particle, the cholesterol is about 5-45 weight/weight % of total lipid in particle, and 1-(2,3-bis((9Z,12Z)-octadeca-9,12-dienyloxy)propyl)pyrrolidine is about 5-60 weight/weight % of total lipid in particle.

A further embodiment pertains to a Lipid-Based Particle, wherein the non-cationic lipids are cholesterol and DSPC, the cationic lipid is 1-(2,3-bis((9Z,12Z)-octadeca-9,12-dienyloxy)propyl)pyrrolidine, the PEG-lipid conjugate is 3,6,9,12,15,18,21,24,27,30,33,36,39,42,45,48,51,54,57,60,63,66,69,72,75,78,81,84,87,90, 93,96,99,102,105,108,111,114,117,120,123,126,129,132,135,138-hexatetracontaoxanonatriacontahect-1-yl 4-{[3,4-bis(hexadecyloxy)butyl]amino}-4-oxobutanoate, and the therapeutic agent is siRNA.

A further embodiment pertains to a Lipid-Based Particle, wherein the 3,6,9,12,15,18,21,24,27,30,33,36,39,42,45,48,51,54,57,60,63,66,69,72,75,78,81,84,87,90, 93,96,99,102,105,108,111,114,117,120,123,126,129,132,135,138-hexatetracontaoxanonatriacontahect-1-yl 4-{[3,4-bis(hexadecyloxy)butyl]amino}-4-oxobutanoate is about 1-25 weight/weight % of total lipid in particle, the DSPC is about 1-30 weight/weight % of total lipid in particle, the cholesterol is about 5-45 weight/weight % of total lipid in particle, and the 1-(2,3-bis((9Z,12Z)-octadeca-9,12-dienyloxy)propyl)pyrrolidine is about 5-60 weight/weight % of total lipid in particle.

A further embodiment pertains to a pharmaceutical composition, wherein the Lipid-Based Particle comprises cholesterol, DSPC, 1-(2,3-bis((9Z,12Z)-octadeca-9,12-dienyloxy)propyl)pyrrolidine, N-[4-(decyloxy)-3-(octadecyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide and one or more nucleic acids.

A further embodiment pertains to a pharmaceutical composition, wherein the N-[4-(decyloxy)-3-(octadecyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide is about 1-25 weight/weight % of total lipid in particle, the DSPC is about 1-30 weight/weight % of total lipid in particle, the cholesterol is about 5-45 weight/weight % of total lipid in particle, and 1-(2,3-bis((9Z,12Z)-octadeca-9,12-dienyloxy)propyl)pyrrolidine is about 5-60 weight/weight % of total lipid in particle.

A further embodiment pertains to a Lipid-Based Particle, wherein the non-cationic lipids are cholesterol and DSPC, the cationic lipid is 1-(2,3-bis((9Z,12Z)-octadeca-9,12-dienyloxy)propyl)pyrrolidine, the PEG-lipid conjugate is N-[4-(decyloxy)-3-(octadecyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide, and the therapeutic agent is siRNA.

A further embodiment pertains to a Lipid-Based Particle, wherein the N-[4-(decyloxy)-3-(octadecyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide is about 1-25 weight/weight % of total lipid in particle, the DSPC is about 1-30 weight/weight % of total lipid in particle, the cholesterol is about 5-45 weight/weight % of total lipid in particle, and the 1-(2,3-bis((9Z,12Z)-octadeca-9,12-dienyloxy)propyl)pyrrolidine is about 5-60 weight/weight % of total lipid in particle.

A further embodiment pertains to a pharmaceutical composition, wherein the Lipid-Based Particle comprises cholesterol, DSPC, 1-(2,3-bis((9Z,12Z)-octadeca-9,12-dienyloxy)propyl)pyrrolidine, N-[3-(octadecyloxy)-4-(tetradecyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide and one or more nucleic acids.

A further embodiment pertains to a pharmaceutical composition, wherein the N-[3-(octadecyloxy)-4-(tetradecyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide is about 1-25 weight/weight % of total lipid in particle, the DSPC is about 1-30 weight/weight % of total lipid in particle, the cholesterol is about 5-45 weight/weight % of total lipid in particle, and 1-(2,3-bis((9Z,12Z)-octadeca-9,12-dienyloxy)propyl)pyrrolidine is about 5-60 weight/weight % of total lipid in particle.

A further embodiment pertains to a Lipid-Based Particle, wherein the non-cationic lipids are cholesterol and DSPC, the cationic lipid is 1-(2,3-bis((9Z,12Z)-octadeca-9,12-dienyloxy)propyl)pyrrolidine, the PEG-lipid conjugate is N-[3-(octadecyloxy)-4-(tetradecyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide, and the therapeutic agent is siRNA.

A further embodiment pertains to a Lipid-Based Particle, wherein the N-[3-(octadecyloxy)-4-(tetradecyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide is about 1-25 weight/weight % of total lipid in particle, the DSPC is about 1-30 weight/weight % of total lipid in particle, the cholesterol is about 5-45 weight/weight % of total lipid in particle, and the 1-(2,3-bis((9Z,12Z)-octadeca-9,12-dienyloxy)propyl)pyrrolidine is about 5-60 weight/weight % of total lipid in particle.

A further embodiment pertains to a pharmaceutical composition, wherein the Lipid-Based Particle comprises cholesterol, DSPC, 1-(2,3-bis((9Z,12Z)-octadeca-9,12-dienyloxy)propyl)pyrrolidine, N-[3,4-bis(hexadecyloxy)butyl]-2,5,8,11,14,17,20,23,26,29,32,35,38,41,44,47,50,53,56,59,62,65,68-tricosaoxaheptacontan-70-amide and one or more nucleic acids.

A further embodiment pertains to a pharmaceutical composition, wherein the N-[3,4-bis(hexadecyloxy)butyl]-2,5,8,11,14,17,20,23,26,29,32,35,38,41,44,47,50,53,56,59,62,65,68-tricosaoxaheptacontan-70-amide is about 1-25 weight/weight % of total lipid in particle, the DSPC is about 1-30 weight/weight % of total lipid in particle, the cholesterol is about 5-45 weight/weight % of total lipid in particle, and 1-(2,3-bis((9Z,12Z)-octadeca-9,12-dienyloxy)propyl)pyrrolidine is about 5-60 weight/weight % of total lipid in particle.

A further embodiment pertains to a Lipid-Based Particle, wherein the non-cationic lipids are cholesterol and DSPC, the cationic lipid is 1-(2,3-bis((9Z,12Z)-octadeca-9,12-dienyloxy)propyl)pyrrolidine, the PEG-lipid conjugate is N-[3,4-bis(hexadecyloxy)butyl]-2,5,8,11,14,17,20,23,26,29,32,35,38,41,44,47,50,53,56,59,62,65,68-tricosaoxaheptacontan-70-amide, and the therapeutic agent is siRNA.

A further embodiment pertains to a Lipid-Based Particle, wherein the N-[3,4-bis(hexadecyloxy)butyl]-2,5,8,11,14,17,20,23,26,29,32,35,38,41,44,47,50,53,56,59,62,65,68-tricosaoxaheptacontan-70-amide is about 1-25 weight/weight % of total lipid in particle, the DSPC is about 1-30 weight/weight % of total lipid in particle, the cholesterol is about 5-45 weight/weight % of total lipid in particle, and the 1-(2,3-bis((9Z,12Z)-octadeca-9,12-dienyloxy)propyl)pyrrolidine is about 5-60 weight/weight % of total lipid in particle.

A further embodiment pertains to a pharmaceutical composition, wherein the Lipid-Based Particle comprises cholesterol, DSPC, 1-(2,3-bis((9Z,12Z)-octadeca-9,12-dienyloxy)propyl)pyrrolidine, N-[3,4-bis(hexadecyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137,140,143,146,149,152,155, 158,161,164,167,170,173,176,179,182,185,188,191,194,197,200,203,206,209,212,215, 218,221,224,227,230,233,236,239,242,245,248,251,254,257,260,263,266,269,272,275,278, 281,284,287,290,293,296,299,302,305,308,311,314,317,320,323,326,329,332,335,338-113oxa340n-340-amide and one or more nucleic acids.

A further embodiment pertains to a pharmaceutical composition, wherein the N-[3,4-bis(hexadecyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137,140,143,146,149,152,155, 158,161,164,167,170,173,176,179,182,185,188,191,194,197,200,203,206,209,212,215, 218,221,224,227,230,233,236,239,242,245,248,251,254,257,260,263,266,269,272,275,278, 281,284,287,290,293,296,299,302,305,308,311,314,317,320,323,326,329,332,335,338-113oxa340n-340-amide is about 1-25 weight/weight % of total lipid in particle, the DSPC is about 1-30 weight/weight % of total lipid in particle, the cholesterol is about 5-45 weight/weight % of total lipid in particle, and 1-(2,3-bis((9Z,12Z)-octadeca-9,12-dienyloxy)propyl)pyrrolidine is about 5-60 weight/weight % of total lipid in particle.

A further embodiment pertains to a Lipid-Based Particle, wherein the non-cationic lipids are cholesterol and DSPC, the cationic lipid is 1-(2,3-bis((9Z,12Z)-octadeca-9,12-dienyloxy)propyl)pyrrolidine, the PEG-lipid conjugate is N-[3,4-bis(hexadecyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137,140,143,146,149,152,155, 158,161,164,167,170,173,176,179,182,185,188,191,194,197,200,203,206,209,212,215, 218,221,224,227,230,233,236,239,242,245,248,251,254,257,260,263,266,269,272,275,278, 281,284,287,290,293,296,299,302,305,308,311,314,317,320,323,326,329,332,335,338-113oxa340n-340-amide, and the therapeutic agent is siRNA.

A further embodiment pertains to a Lipid-Based Particle, wherein the N-[3,4-bis(hexadecyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137,140,143,146,149,152,155, 158,161,164,167,170,173,176,179,182,185,188,191,194,197,200,203,206,209,212,215, 218,221,224,227,230,233,236,239,242,245,248,251,254,257,260,263,266,269,272,275,278, 281,284,287,290,293,296,299,302,305,308,311,314,317,320,323,326,329,332,335,338-113oxa340n-340-amide is about 1-25 weight/weight % of total lipid in particle, the DSPC is about 1-30 weight/weight % of total lipid in particle, the cholesterol is about 5-45 weight/weight % of total lipid in particle, and the 1-(2,3-bis((9Z,12Z)-octadeca-9,12-dienyloxy)propyl)pyrrolidine is about 5-60 weight/weight % of total lipid in particle.

A further embodiment pertains to a pharmaceutical composition, wherein the Lipid-Based Particle comprises cholesterol, DSPC, 1-(2,3-bis((9Z,12Z)-octadeca-9,12-dienyloxy)propyl)pyrrolidine, N-[4-(hexadecyloxy)-3-(octadecyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide and one or more nucleic acids.

A further embodiment pertains to a pharmaceutical composition, wherein the N-[4-(hexadecyloxy)-3-(octadecyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide is about 1-25 weight/weight % of total lipid in particle, the DSPC is about 1-30 weight/weight % of total lipid in particle, the cholesterol is about 5-45 weight/weight % of total lipid in particle, and 1-(2,3-bis((9Z,12Z)-octadeca-9,12-dienyloxy)propyl)pyrrolidine is about 5-60 weight/weight % of total lipid in particle.

A further embodiment pertains to a Lipid-Based Particle, wherein the non-cationic lipids are cholesterol and DSPC, the cationic lipid is 1-(2,3-bis((9Z,12Z)-octadeca-9,12-dienyloxy)propyl)pyrrolidine, the PEG-lipid conjugate is N-[4-(hexadecyloxy)-3-(octadecyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide, and the therapeutic agent is siRNA.

A further embodiment pertains to a Lipid-Based Particle, wherein the N-[4-(hexadecyloxy)-3-(octadecyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide is about 1-25 weight/weight % of total lipid in particle, the DSPC is about 1-30 weight/weight % of total lipid in particle, the cholesterol is about 5-45 weight/weight % of total lipid in particle, and the 1-(2,3-bis((9Z,12Z)-octadeca-9,12-dienyloxy)propyl)pyrrolidine is about 5-60 weight/weight % of total lipid in particle.

A further embodiment pertains to a Lipid-Based Particle, wherein the non-cationic lipids are cholesterol and DSPC, the cationic lipid is 1-(2,3-bis((9Z,12Z)-octadeca-9,12-dienyloxy)propyl)pyrrolidine, the PEG-lipid conjugate is N-[3,4-bis(hexadecyloxy)butyl]-N′-3,6,9,12,15,18,21,24,27,30,33,36,39,42,45,48,51,54,57,60,63,66,69,72,75,78,81,84,87,90, 93,96,99,102,105,108,111,114,117,120,123,126,129,132,135,138-hexatetracontaoxanonatriacontahect-1-ylsuccinamide, and the therapeutic agent is siRNA.

A further embodiment pertains to a Lipid-Based Particle, wherein the N-[3,4-bis(hexadecyloxy)butyl]-N′-3,6,9,12,15,18,21,24,27,30,33,36,39,42,45,48,51,54,57,60,63,66,69,72,75,78,81,84,87,90, 93,96,99,102,105,108,111,114,117,120,123,126,129,132,135,138-hexatetracontaoxanonatriacontahect-1-ylsuccinamide is about 0.1-20 weight/weight % of total lipid in particle, the DSPC is about 1-30 weight/weight % of total lipid in particle, the cholesterol is about 5-45 weight/weight % of total lipid in particle, and the 1-(2,3-bis((9Z,12Z)-octadeca-9,12-dienyloxy)propyl)pyrrolidine is about 5-60 weight/weight % of total lipid in particle.

A further embodiment pertains to a Lipid-Based Particle, wherein the non-cationic lipids are cholesterol and DSPC, the cationic lipid is 1-(2,3-bis((9Z,12Z)-octadeca-9,12-dienyloxy)propyl)pyrrolidine, the PEG-lipid conjugate is N-[3-(octadecyloxy)-4-(tetradecyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide, and the therapeutic agent is siRNA.

A further embodiment pertains to a Lipid-Based Particle, wherein the N-[3-(octadecyloxy)-4-(tetradecyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide is about 0.1-20 weight/weight % of total lipid in particle, the DSPC is about 1-30 weight/weight % of total lipid in particle, the cholesterol is about 5-45 weight/weight % of total lipid in particle, and the 1-(2,3-bis((9Z,12Z)-octadeca-9,12-dienyloxy)propyl)pyrrolidine is about 5-60 weight/weight % of total lipid in particle.

A further embodiment pertains to a Lipid-Based Particle, wherein the non-cationic lipids are cholesterol and DSPC, the cationic lipid is 1-(2,3-bis((9Z,12Z)-octadeca-9,12-dienyloxy)propyl)pyrrolidine, the PEG-lipid conjugate is N-[3,4-bis(hexadecyloxy)butyl]-2,5,8,11,14,17,20,23,26,29,32,35,38,41,44,47,50,53,56,59,62,65,68-tricosaoxaheptacontan-70-amide, and the therapeutic agent is siRNA.

A further embodiment pertains to a Lipid-Based Particle, wherein the N-[3,4-bis(hexadecyloxy)butyl]-2,5,8,11,14,17,20,23,26,29,32,35,38,41,44,47,50,53,56,59,62,65,68-tricosaoxaheptacontan-70-amide is about 0.1-20 weight/weight % of total lipid in particle, the DSPC is about 1-30 weight/weight % of total lipid in particle, the cholesterol is about 5-45 weight/weight % of total lipid in particle, and the 1-(2,3-bis((9Z,12Z)-octadeca-9,12-dienyloxy)propyl)pyrrolidine is about 5-60 weight/weight % of total lipid in particle.

A further embodiment pertains to functional CaBLES comprising one or more (PEG)-lipid conjugates of Formula I, one or more non-cationic lipids, and one or more cationic lipids which effectively encapsulate nucleic acids, such as siRNA, with efficiencies from about 50-100%.

A further embodiment pertains to functional CaBLES comprising one or more (PEG)-lipid conjugates of Formula I, one or more non-cationic lipids, and one or more cationic lipids which effectively encapsulate nucleic acids, such as siRNA, with efficiencies from about 80-100%.

A further embodiment pertains to a Lipid-Based Particle, wherein the ratio of one or more (PEG)-lipid conjugates, one or more non-cationic lipids, and one or more cationic lipids of claim 1, to one or more therapeutic agents is between about 50:1 to about 5:1.

A further embodiment pertains to a Lipid-Based Particle, wherein the ratio of one or more (PEG)-lipid conjugates, one or more non-cationic lipids, and one or more cationic lipids of claim 1, to one or more therapeutic agents is between about 30:1 to about 10:1.

A further embodiment pertains to examples of non-cationic lipids that are useful for the practice of this invention which include, but are not limited to, cholesterol, cholesterol sulfate, ceramide, sphingomyelin, lecithin, sphingomyelin, egg sphingomyelin, milk sphingomyelin; egg phosphatidylcholine, hydrogenated egg phosphatidylcholine, hydrogenated soybean phosphatidylethanolamine, egg phosphatidylethanolamine, hydrogenated soybean phosphatidylcholine, soybean phosphatidylcholine, 1,2-dilauroyl-sn-glycerol, 1,2-dimyristoyl-sn-glycerol, 1,2-dipalmitoyl-sn-glycerol, 1,2-distearoyl-sn-glycerol, 1,2-dilauroyl-sn-glycero-3-phosphatidic acid, 1,2-dimyristoyl-sn-glycero-3-phosphatidic acid, 1,2-dipalmitoyl-sn-glycero-3-phosphatidic acid, 1,2-distearoyl-sn-glycero-3-phosphatidic acid, 1,2-diarachidoyl-sn-glycero-3-phosphocholine, 1,2-dilauroyl-sn-glycero-3-phosphocholine, 1,2-dimyristoyl-sn-glycero-3-phosphocholine, dioleoylphosphatidylcholine, 1,2-dierucoyl-sn-glycero-3-phosphocholine, 1-myristoyl-2-palmitoyl-sn-glycero-3-phosphocholine, 1-myristoyl-2-stearoyl-sn-glycero-3-phosphocholine, 1-palmitoyl-2-myristoyl-sn-glycero-3-phosphocholine, 1-palmitoyl-2-stearoyl-sn-glycero-3-phosphocholine, 1-stearoyl-2-myristoyl-sn-glycero-3-phosphocholine, 1-stearoyl-2-palmitoyl-sn-glycero-3-phosphocholine, 1-myristoyl-2-oleoyl-sn-glycero-3-phosphocholine, 1-palmitoyl-2-oleoyl-sn-glycero-3-phosphocholine; 1-stearoyl-2-oleoyl-sn-glycero-3-phosphocholine, 1-myristoyl-2-lyso-sn-glycero-3-phosphocholine, 1-palmitoyl-2-lyso-sn-glycero-3-phosphocholine, 1-stearoyl-2-lyso-sn-glycero-3-phosphocholine, 1,2-dipalmitoyl-sn-glycero-O-ethyl-3-phosphocholine, 1,2-dipalmitoyl-sn-glycero-3-phosphocholine; 1,2-distearoyl-sn-glycero-3-phosphocholine; 1-palmitoyl-2-linoleoyl-sn-glycero-3-phosphocholine, dioleoylphosphatidylethanolamine, palmitoyloleoyl-phosphatidylethanolamine, dioleoylphosphatidylglycerol-1,2-dilauroyl-sn-glycero-3-phosphoethanolamine, 1,2-dimyristoyl-sn-glycero-3-phosphoethanolamine, 1,2-dipalmitoyl-sn-glycero-3-phosphoethanolamine, 1,2-distearoyl-sn-glycero-3-phosphoethanolamine, 1,2-dioleoyl-sn-glycero-3-phosphoethanolamine, 1,2-dilauroyl-sn-glycero-3-phosphoglycerol, 1,2-dimyristoyl-sn-glycero-3-phosphoglycerol-1,2-dimyristoyl-sn-glycero-3-phospho-sn-1-glycerol, 1,2-dipalmitoyl-sn-glycero-3-phosphoglycerol, 1,2-distearoyl-sn-glycero-3-phosphoglycero, 1,2-distearoyl-sn-glycero-3-phospho-sn-1-glycerol, 1-palmitoyl-2-oleoyl-sn-glycero-3-phosphoglycerol, 1-palmitoyl-2-oleoyl-sn-glycero-3-phosphoglycerol, 1,2-dipalmitoyl-sn-glycero-3-phospho-L-serine, 1,2-dimyristoyl-sn-glycero-3-phospho-L-serine, 1,2-dipalmitoyl-sn-glycero-3-phospho-L-serine, 1,2-distearoyl-sn-glycero-3-phospho-L-serine, 1,2-dioleoyl-sn-glycero-3-phospho-L-serine, and 1-palmitoyl-2-oleoyl-sn-glycero-3-phospho-L-serine or a mixture thereof.

A further embodiment pertains to examples of cationic lipids that are useful for the practice of this invention which include, but are not limited to, N,N-dioleyl-N,N-dimethylammonium chloride, DC-Chol; 1,3-dioleoyloxy-2-(6-carboxyspermyl)-propyl amide, dioctadecylamidoglycyl spermine, N,N-distearyl-N,N-dimethylammonium bromide, N-(2,3-dioleyloxy)propyl)-N,N-dimethylammonium chloride, 1,2-dioleoyl-3-trimethylammonium-propane chloride, 1,2-dilineoyl-3-dimethylammonium-propane, N-(1-(2,3-dioleyloxy)propyl)-N,N,N-trimethylammonium chloride, 1,2-dioleoyl-3-dimethylammonium propane, 1,2-distearyloxy-N,N-dimethyl-3-aminopropane; didodecyldimethylammonium bromide, dioleoyloxy-N-(2-sperminecarboxamido)ethyl)-N,N-dimethyl-1-propanaminiumtrifluoroacetate, 1,2-dimyristyloxypropyl-3-dimethyl-hydroxyethyl ammonium bromide, 1,2-dioleoylcarbamyl-3-dimethylammoniumpropane, tetramethyltetrapalmitoyl spermine, tetramethyltetraoleyl spermine, tetramethyldioleyl spermine, tetramethyltetramyristyl spermine, tetramethyltetralauryl spermine, 1-(2-((9Z,12Z)-octadeca-9,12-dienyloxy)-1-(((9Z,12Z)-octadeca-9,12-dienyloxy)methyl)ethyl)pyrrolidine; N,N-dimethyl-N-(2-((9Z,12Z)-octadeca-9,12-dienyloxy)-1-(((9Z,12Z)-octadeca-9,12-dienyloxy)methyl)ethyl)amine; N-(3-(1H-imidazol-1-yl)propyl)-N-(2((9Z,12Z)-octadeca-9,12-dienyloxy)-1-(((9Z,12Z)-octadeca-9,12-dienyloxy)methyl)ethyl)amine; 1-methyl-4-(2((9Z,12Z)-octadeca-9,12-dienyloxy)-1-(((9Z,12Z)-octadeca-9,12-dienyloxy)methyl)ethyl)piperazine; 4-(2-((9Z,12Z)-octadeca-9,12-dienyloxy)-1-(((9Z,12Z)-octadeca-9,12-dienyloxy)methyl)ethyl)morpholine; N-(2-((9Z,12Z)-octadeca-9,12-dienyloxy)-1-(((9Z,12Z)-octadeca-9,12-dienyloxy)methyl)ethyl)-N-(3-pyrrolidin-1-ylpropyl)amine; N,N-dimethyl-N′-(2-((9Z,12Z)-octadeca-9,12-dienyloxy)-1-(((9Z,12Z)-octadeca-9,12-dienyloxy)methyl)ethyl)ethane-1,2-diamine; N-(2-(4-methylpiperazin-1-yl)ethyl)-N-(2-((9Z,12Z)-octadeca-9,12-dienyloxy)-1-(((9Z,12Z)-octadeca-9,12-dienyloxy)methyl)ethyl)amine; N-(2-(1H-imidazol-4-yl)ethyl)-N-(2-((9Z,12Z)-octadeca-9,12-dienyloxy)-1-(((9Z,12Z)-octadeca-9,12-dienyloxy)methyl)ethyl)amine; N,N-dimethyl-N-(3-(4-(2-((9Z,12Z)-octadeca-9,12-dienyloxy)-1-(((9Z,12Z)-octadeca-9,12-dienyloxy)methyl)ethyl)piperazin-1-yl)propyl)amine; 1,3-bis((9Z,12Z)-octadeca-9,12-dienyloxy)propan-2-amine; N-((1-methylpiperidin-4-yl)methyl)-N-(2((9Z,12Z)-octadeca-9,12-dienyloxy)-1-(((9Z,12Z)-octadeca-9,12-dienyloxy)methyl)ethyl)amine; N-(2-((9Z,12Z)-octadeca-9,12-dienyloxy)-1-(((9Z,12Z)-octadeca-9,12-dienyloxy)methyl)ethyl)-N-(3-(pyrrolidin-1-ylmethyl)benzyl)amine; N-methyl-N-(2-((9Z,12Z)-octadeca-9,12-dienyloxy)-1-(((9Z,12Z)-octadeca-9,12-dienyloxy)methyl)ethyl)-N-(3-pyrrolidin-1-ylpropyl)amine; N-(3-((4-methylpiperazin-1-yl)methyl)benzyl)-N-(2((9Z,12Z)-octadeca-9,12-dienyloxy)-1-(((9Z,12Z)-octadeca-9,12-dienyloxy)methyl)ethyl)amine; N-methyl-N-((1-methylpiperidin-4-yl)methyl)-N-(2-((9Z,12Z)-octadeca-9,12-dienyloxy)-1-(((9Z,12Z)-octadeca-9,12-dienyloxy)methyl)ethyl)amine; N,N,N′-trimethyl-N′-(2-((9Z,12Z)-octadeca-9,12-dienyloxy)-1-(((9Z,12Z)-octadeca-9,12-dienyloxy)methyl)ethyl)propane-1,3-diamine; N-methyl-N-(2-((9Z,12Z)-octadeca-9,12-dienyloxy)-1-(((9Z,12Z)-octadeca-9,12-dienyloxy)methyl)ethyl)-N-(3-(pyrrolidin-1-ylmethyl)benzyl)amine; 1-(2-(1H-imidazol-1-yl)ethyl)-4-(2-((9Z,12Z)-octadeca-9,12-dienyloxy)-1-(((9Z,12Z)-octadeca-9,12-dienyloxy)methyl)ethyl)piperazine; N-(2-((9Z,12Z)-octadeca-9,12-dienyloxy)-1-(((9Z,12Z)-octadeca-9,12-dienyloxy)methyl)ethyl)-N-((2-pyrrolidin-1-ylpyridin-3-yl)methyl)amine; (9Z,9′Z,12Z,12′Z)-2-(4-methylpiperazin-1-yl)propane-1,3-diyl dioctadeca-9,12-dienoate; (9Z,9′Z,12Z,12′Z)-2-(3-(pyrrolidin-1-yl)propylamino)propane-1,3-diyl dioctadeca-9,12-dienoate; 1-methyl-4-(3((9Z,12Z)-octadeca-9,12-dienyloxy)-2-(((9Z,12Z)-octadeca-9,12-dienyloxy)methyl)propyl)piperazine; 1-(3-((9Z,12Z)-octadeca-9,12-dienyloxy)-2-(((9Z,12Z)-octadeca-9,12-dienyloxy)methyl)propyl)pyrrolidine; N-(3-aminopropyl)-N′-{3-[(2-[(9Z,12Z)-octadeca-9,12-dienyloxy]-1-{[(9Z,12Z)-octadeca-9,12-dienyloxy]methyl}ethyl)amino]propyl}butane-1,4-diamine; N-(3-[(9Z,12Z)-octadeca-9,12-dienyloxy]-2-{[(9Z,12Z)-octadeca-9,12-dienyloxy]methyl}propyl)-N-(3-pyrrolidin-1-ylpropyl)amine; N,N-dimethyl-N-(3-[(9Z,12Z)-octadeca-9,12-dienyloxy]-2-{[(9Z,12Z)-octadeca-9,12-dienyloxy]methyl}propyl)amine; 3-[(9Z,12Z)-octadeca-9,12-dienyloxy]-2-{[(9Z,12Z)-octadeca-9,12-dienyloxy]methyl}propyl 2-(diethylamino)ethylcarbamate; 3-[(9Z,12Z)-octadeca-9,12-dienyloxy]-2-{[(9Z,12Z)-octadeca-9,12-dienyloxy]methyl}propyl 2-pyrrolidin-1-ylethylcarbamate; 3-[(9Z,12Z)-octadeca-9,12-dienyloxy]-2-{[(9Z,12Z)-octadeca-9,12-dienyloxy]methyl}propyl 2-(dimethylamino)ethylcarbamate; 1-(2-[(9Z,12Z)-octadeca-9,12-dienyloxy]-1-{[(9Z,12Z)-octadeca-9,12-dienyloxy]methyl}ethyl)-4-(2-pyrrolidin-1-ylethyl)piperazine; N-(2-[(9Z)-octadec-9-enyloxy]-1-{[(9Z)-octadec-9-enyloxy]methyl}ethyl)-N-(3-pyrrolidin-1-ylpropyl)amine, 1-(2-[(9Z,12Z)-octadeca-9,12-dienyloxy]-1-{[(9Z,12Z)-octadeca-9,12-dienyloxy]methyl}ethyl)azetidine, 2-methyl-1-(2-[(9Z,12Z)-octadeca-9,12-dienyloxy]-1-{[(9Z,12Z)-octadeca-9,12-dienyloxy]methyl}ethyl)aziridine, 1-{3,4-bis[(9Z,12Z)-octadeca-9,12-dienyloxy]butyl}piperidine, 4-{3,4-bis[(9Z,12Z)-octadeca-9,12-dienyloxy]butyl}morpholine, N,N-diethyl-3,4-bis[(9Z,12Z)-octadeca-9,12-dienyloxy]butan-1-amine, N,N-dimethyl-3,4-bis[(9Z,12Z)-octadeca-9,12-dienyloxy]butan-1-amine, 1-{3,4-bis[(9Z,12Z)-octadeca-9,12-dienyloxy]butyl}-4-phenylpiperazine, 1-{3,4-bis[(9Z,12Z)-octadeca-9,12-dienyloxy]butyl}-4-methylpiperazine, N-(2-methoxyethyl)-N-methyl-3,4-bis[(9Z,12Z)-octadeca-9,12-dienyloxy]butan-1-amine, 1-{3,4-bis[(9Z,12Z)-octadeca-9,12-dienyloxy]butyl}-4-(2-methoxyphenyl)piperazine, N-{3,4-bis[(9Z,12Z)-octadeca-9,12-dienyloxy]butyl}-N,N′,N′-trimethylethane-1,2-diamine, N-{3,4-bis[(9Z,12Z)-octadeca-9,12-dienyloxy]butyl}-N-methyl-N-(2-pyridin-2-ylethyl)amine, N-benzyl-N-{3,4-bis[(9Z,12Z)-octadeca-9,12-dienyloxy]butyl}-N-methylamine, N-{3,4-bis[(9Z,12Z)-octadeca-9,12-dienyloxy]butyl}-N-(4-fluorobenzyl)-N-methylamine, 1-{3,4-bis[(9Z,12Z)-octadeca-9,12-dienyloxy]butyl}-4-(2-fluorophenyl)piperazine, N-benzyl-N-{3,4-bis[(9Z,12Z)-octadeca-9,12-dienyloxy]butyl}-N-ethylamine, N-{3,4-bis[(9Z,12Z)-octadeca-9,12-dienyloxy]butyl}-N-ethyl-N′,N′-dimethylethane-1,2-diamine, 1-{3,4-bis[(9Z,12Z)-octadeca-9,12-dienyloxy]butyl}-N,N-dimethylpiperidin-4-amine, 1-{3,4-bis[(9Z,12Z)-octadeca-9,12-dienyloxy]butyl}-N,N-dimethylpyrrolidin-3-amine, N,N-bis(2-methoxyethyl)-3,4-bis[(9Z,12Z)-octadeca-9,12-dienyloxy]butan-1-amine, 1-{3,4-bis[(9Z,12Z)-octadeca-9,12-dienyloxy]butyl}-4-methoxypiperidine, 1-{(3R)-3,4-bis[(9Z,12Z)-octadeca-9,12-dienyloxy]butyl}pyrrolidine, 1-{(3S)-3,4-bis[(9Z,12Z)-octadeca-9,12-dienyloxy]butyl}pyrrolidine, N-{(3R)-3,4-bis[(9Z,12Z)-octadeca-9,12-dienyloxy]butyl}-N,N-diethylamine, N-{(3S)-3,4-bis[(9Z,12Z)-octadeca-9,12-dienyloxy]butyl}-N,N-diethylamine, 1-{3,4-bis[(9Z,12Z)-octadeca-9,12-dienyloxy]butyl}pyrrolidine, N-(2-{3,4-bis[(9Z,12Z)-octadeca-9,12-dienyloxy]butoxy}ethyl)-N,N-diethylamine, 2-(2-{3,4-bis[(9Z,12Z)-octadeca-9,12-dienyloxy]butoxy}ethyl)-1-methylpyrrolidine, 1-(2-{3,4-bis[(9Z,12Z)-octadeca-9,12-dienyloxy]butoxy}ethyl)aziridine, 1-(2-{3,4-bis[(9Z,12Z)-octadeca-9,12-dienyloxy]butoxy}ethyl)-4-methylpiperazine, N-(2-{3,4-bis[(9Z,12Z)-octadeca-9,12-dienyloxy]butoxy}ethyl)-N,N-dimethylamine, 4-(diethylamino)-2-[(9Z,12Z)-octadeca-9,12-dienoyloxy]butyl (9Z,12Z)-octadeca-9,12-dienoate, 1-(2-{3,4-bis[(9Z,12Z)-octadeca-9,12-dienyloxy]butoxy}ethyl)pyrrolidine, N,N-diethyl-N-(2-{2-[(8Z,11Z)-heptadeca-8,11-dienyl]-2-[(9Z,12Z)-octadeca-9,12-dienyl]-1,3-dioxolan-4-yl}ethyl)amine, 1-{[(9Z)-octadec-9-enoyloxy]methyl}-3-pyrrolidin-1-ylpropyl (9Z)-octadec-9-enoate, 1-{3,4-bis[(9Z)-octadec-9-enyloxy]butyl}pyrrolidine, 1-{[(5Z,8Z,11Z,14Z)-icosa-5,8,11,14-tetraenoyloxy]methyl}-3-pyrrolidin-1-ylpropyl (5Z,8Z,11Z,14Z)-icosa-5,8,11,14-tetraenoate, (3S)-3,4-bis[(9Z,12Z)-octadeca-9,12-dienyloxy]butyl 3-pyrrolidin-1-ylpropylcarbamate, 1-[3,4-bis(octadecyloxy)butyl]pyrrolidine, 1-[3,4-bis(hexadecyloxy)butyl]pyrrolidine, 1-{3,4-bis[(9E)-hexadec-9-enyloxy]butyl}pyrrolidine, 1-{3,4-bis[(9E)-octadec-9-enyloxy]butyl}pyrrolidine, 1-{3,4-bis[(9E,12E)-octadeca-9,12-dienyloxy]butyl}pyrrolidine, 1-{3,4-bis[(9Z,12Z,15Z)-octadeca-9,12,15-trienyloxy]butyl}pyrrolidine, N¹-{(3S)-3,4-bis[(9Z,12Z)-octadeca-9,12-dienyloxy]butyl}-N³,N³-diethyl-beta-alaninamide, N-{3,4-bis[(9Z,12Z)-octadeca-9,12-dienyloxy]butyl}-N-[3-(1H-imidazol-1-yl)propyl]amine, N-{3,4-bis[(9Z,12Z)-octadeca-9,12-dienyloxy]butyl}-N,N′,N′-trimethylpropane-1,3-diamine, 1-(1-{3,4-bis[(9Z,12Z)-octadeca-9,12-dienyloxy]butyl}pyrrolidin-3-yl)-1H-imidazole, N-{3,4-bis[(9Z,12Z)-octadeca-9,12-dienyloxy]butyl}-N-(3-pyrrolidin-1-ylpropyl)amine, N-{3,4-bis[(9Z,12Z)-octadeca-9,12-dienyloxy]butyl}-N′,N′-dimethylpropane-1,3-diamine, 1-{3,4-bis[(9Z,12Z)-octadeca-9,12-dienyloxy]butyl}azetidine, 1-{3,4-bis[(9Z,12Z)-octadeca-9,12-dienyloxy]butyl}-2-methylpyrrolidine, 1-{3,4-bis[(9Z,12Z)-octadeca-9,12-dienyloxy]butyl}-2,5-dimethylpyrrolidine, are 1-(2,3-bis((9Z,12Z)-octadeca-9,12-dienyloxy)propyl)pyrrolidine, 1-(2,3-bis((9Z,12Z)-octadeca-9,12-dienyloxy)propyl)-1H-imidazole, 1-(2,3-bis((9Z,12Z)-octadeca-9,12-dienyloxy)propyl)-4-methylpiperazine, 1-(2,3-bis((9Z,12Z)-octadeca-9,12-dienyloxy)propyl)-4-methyl-1,4-diazepane, 1-(2,3-bis((9Z,12Z)-octadeca-9,12-dienyloxy)propyl)-4-phenylpiperazine, 1-(2,3-bis((9Z,12Z)-octadeca-9,12-dienyloxy)propyl)-4-pyridin-2-ylpiperazine, 1-(2,3-bis((9Z,12Z)-octadeca-9,12-dienyloxy)propyl)piperidine, 4-(2,3-bis((9Z,12Z)-octadeca-9,12-dienyloxy)propyl)morpholine, 1-((2R)-2,3-bis((9Z,12Z)-octadeca-9,12-dienyloxy)propyl)pyrrolidine, 1-((2S)-2,3-bis((9Z,12Z)-octadeca-9,12-dienyloxy)propyl)pyrrolidine, 1-(2,3-bis((9Z,12Z)-octadeca-9,12-dienyloxy)propyl)-4-ethylpiperazine, N-(2,3-bis((9Z,12Z)-octadeca-9,12-dienyloxy)propyl)-N-methyl-N-(3-(pyrrolidin-1-ylmethyl)benzyl)amine, N-(2-(4-(2,3-bis((9Z,12Z)-octadeca-9,12-dienyloxy)propyl)piperazin-1-yl)ethyl)-N,N-dimethylamine, 1-((2S)-2,3-bis((9Z,12Z)-octadeca-9,12-dienyloxy)propyl)-4-methylpiperazine, 1-((2R)-2,3-bis((9Z,12Z)-octadeca-9,12-dienyloxy)propyl)-4-methylpiperazine, 1-(2,3-bis((9Z,12Z)-octadeca-9,12-dienyloxy)propyl)-4-(2-pyrrolidin-1-ylethyl)piperazine, 2-(4-(2,3-bis((9Z,12Z)-octadeca-9,12-dienyloxy)propyl)piperazin-1-yl)pyrimidine, 1-(2,3-bis((9Z,12Z)-octadeca-9,12-dienyloxy)propyl)-N,N-diethylpyrrolidin-3-amine, 1-((9Z,12Z)-octadeca-9,12-dienyloxy)-3-pyrrolidin-1-ylpropan-2-ol, 2-[(9Z,12Z)-octadeca-9,12-dienoyloxy]-1-(pyrrolidin-1-ylmethyl)ethyl (9Z,12Z)-octadeca-9,12-dienoate, 2-[(9Z,12Z)-octadeca-9,12-dienyloxy]-1-(pyrrolidin-1-ylmethyl)ethyl (9Z,12Z)-octadeca-9,12-dienoate, 1-({2-[(8Z,11Z)-heptadeca-8,11-dienyl]-2-[(9Z,12Z)-octadeca-9,12-dienyl]-1,3-dioxolan-4-yl}methyl)pyrrolidine, 1-{2,3-bis[(5Z,8Z,11Z,14Z)-icosa-5,8,11,14-tetraenyloxy]propyl}pyrrolidine, 1-{3-[(5Z,8Z,11Z,14Z)-icosa-5,8,11,14-tetraenyloxy]-2-[(9Z,12Z)-octadeca-9,12-dienyloxy]propyl}pyrrolidine, 1-{2,3-bis[(9E,12E)-octadeca-9,12-dienyloxy]propyl}pyrrolidine, 1-{2-[(9E,12E)-octadeca-9,12-dienyloxy]-3-[(9Z,12Z)-octadeca-9,12-dienyloxy]propyl}pyrrolidine, 1-[2,3-bis(tetradecyloxy)propyl]pyrrolidine, 1-[2,3-bis(octadecyloxy)propyl]pyrrolidine, 1-{2,3-bis[(9Z)-octadec-9-enyloxy]propyl}pyrrolidine, 1-[2,3-bis(dodecyloxy)propyl]pyrrolidine, 1-{2,3-bis[(9Z,12Z)-octadeca-9,12-dienyloxy]propyl}pyrrolidin-3-ol, 1-{3-[(9Z,12Z)-hexadeca-9,12-dienyloxy]-2-[(9Z)-octadec-9-enyloxy]propyl}pyrrolidine, 1-{2,3-bis[(9Z,12Z)-octadeca-9,12-dienyloxy]propyl}-N,N-dimethylpyrrolidin-3-amine and 1-[3-[(9Z,12Z)-hexadeca-9,12-dienyloxy]-2-(tetradecyloxy)propyl]pyrrolidine.

Cationic lipids are described in, e.g., U.S. application Ser. No. 12/425,198, which was filed on Apr. 16, 2009, and is incorporated herein by reference.

Cationic lipids are described in, e.g., U.S. application Ser. No. 12/425,266, which was filed on Apr. 16, 2009, and is incorporated herein by reference.

Cationic lipids are described in, e.g., U.S. application Ser. No. 12/425,254, which was filed on Apr. 16, 2009, and is incorporated herein by reference.

In still a further embodiment, the cationic lipids of the CaBLES and Lipid-Based Particles comprises about 2 to about 60 weight/weight percent of total lipid in the particle.

In still a further embodiment, the non-cationic lipids of the Cables and Lipid-Based Particles comprises about 5 to about 90 weight/weight percent of total lipid in the particle.

In still a further embodiment, the PEG-lipid conjugates of the CaBLES and Lipid-Based Particles comprises from 0.1 to about 20 weight/weight percent of total lipid in the particle.

### Methods of Treatment and Methods of Making Lipid-Based Particles

Still another embodiment pertains to a method of treating cancer in a mammal comprising administering thereto a Lipid-Based Particle.

Still another embodiment comprises methods of treating cancer in a mammal comprising administering thereto a Lipid-Based Particle comprising one or more polyethylene glycol-lipid conjugates having Formula (I)

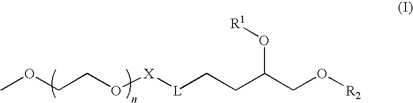

wherein
R¹ and R² are independently R³, or C(O)R³; or
R¹ and R² together are C(R³)₂;
R³ is C₁₂-C₂₄ alkyl;
L is C(OCH₃)₂, NHC(O), C(O)NH, OC(O)NH, NHC(O)O, NHC(O)NH, N(N)C(O), C(O)N(N), SS, NHC(O)L²C(O)O, NHC(O)L²C(O)NH, OC(O)L²C(O)O, OC(O)L²C(O)NH, C(O)O, OC(O), S, O, CH₂CH(═N)NHR⁴C(O), or C(═NNHCH₃)R⁴;
R⁴ is aryl or heteroaryl;
L² is C₁-C₆ alkyl;
X is a bond or C₁-C₆ alkyl; and
n is 10-200; and
one or more non-cationic lipids, one or more cationic lipids, and one or more therapeutic agents.

A further embodiment pertains to a method of making CaBLES or Lipid-Based Particles, comprising: (a) mixing the cationic lipid(s), the non-cationic lipid(s) and the PEG-lipid conjugate(s); (b) adding the mixture of step (a) to one or more therapeutic agents; and (c) separating and purifying resulting suspension of step (b).

A further embodiment pertains to a method of making Lipid-Based Particles wherein the mixture of step (a) and one or more said therapeutic agents are warmed to about 60° C. prior to the addition of the mixture of step (a) to one or more therapeutic agents via needle injection.

### Pharmaceutical Compositions and Methods of Administration

Therapeutically effective amounts of Lipid-Based Particles of this invention depend on recipient of treatment, disease treated and severity thereof, composition comprising it, time of administration, route of administration, duration of treatment, potency, rate of clearance and whether or not another drug is co-administered. The amount of Lipid-Based Particles of this invention used to make compositions to be administered daily to a patient in a single dose or in divided doses is from about 0.001 to about 200 mg/kg body weight. Single dose compositions contain these amounts or a combination of submultiples thereof.

One embodiment pertains to a pharmaceutical composition comprising one or more (PEG)-lipid conjugates of Formula 1, one or more non-cationic lipids, one or more cationic lipids, one or more therapeutic agents, and a pharmaceutically acceptable excipient.

Lipid-Based Particles of this invention may be administered, for example, bucally, ophthalmically, orally, osmotically, parenterally (intramuscularly, intraperitoneally intrasternally, intravenously, subcutaneously), rectally, topically, transdermally, vaginally and intraarterially as well as by intraarticular injection, infusion, and placement in the body, such as, for example, the vasculature.

Lipid-Based Particles may be administered with or without an excipient. Excipients include, but are not limited to, encapsulators and additives such as absorption accelerators, antioxidants, binders, buffers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents, mixtures thereof and the like.

Excipients for preparation of compositions comprising Lipid-Based Particles to be administered orally include, but are not limited to, agar, alginic acid, aluminum hydroxide, benzyl alcohol, benzyl benzoate, 1,3-butylene glycol, carbomers, castor oil, cellulose, cellulose acetate, cocoa butter, corn starch, corn oil, cottonseed oil, cross-povidone, diglycerides, ethanol, ethyl cellulose, ethyl laureate, ethyl oleate, fatty acid esters, gelatin, germ oil, glucose, glycerol, groundnut oil, hydroxypropylmethyl celluose, isopropanol, isotonic saline, lactose, magnesium hydroxide, magnesium stearate, malt, mannitol, monoglycerides, olive oil, peanut oil, potassium phosphate salts, potato starch, povidone, propylene glycol, Ringer's solution, safflower oil, sesame oil, sodium carboxymethyl cellulose, sodium phosphate salts, sodium lauryl sulfate, sodium sorbitol, soybean oil, stearic acids, stearyl fumarate, sucrose, surfactants, talc, tragacanth, tetrahydrofurfuryl alcohol, triglycerides, water, mixtures thereof and the like. Excipients for preparation of compositions comprising a compound having formula (I) to be administered ophthalmically or orally include, but are not limited to, 1,3-butylene glycol, castor oil, corn oil, cottonseed oil, ethanol, fatty acid esters of sorbitan, germ oil, groundnut oil, glycerol, isopropanol, olive oil, polyethylene glycols, propylene glycol, sesame oil, water, mixtures thereof and the like. Excipients for preparation of compositions comprising a compound having formula (I) to be administered osmotically include, but are not limited to, chlorofluorohydrocarbons, ethanol, water, mixtures thereof and the like. Excipients for preparation of compositions comprising a compound having formula (I) to be administered parenterally include, but are not limited to, 1,3-butanediol, castor oil, corn oil, cottonseed oil, dextrose, germ oil, groundnut oil, liposomes, oleic acid, olive oil, peanut oil, Ringer's solution, safflower oil, sesame oil, soybean oil, U.S.P. or isotonic sodium chloride solution, water, mixtures thereof and the like. Excipients for preparation of compositions comprising a compound having formula (I) to be administered rectally or vaginally include, but are not limited to, cocoa butter, polyethylene glycol, wax, mixtures thereof and the like.

### Combination Therapy

The present invention further provides methods of using a compound, formulation, or composition of the invention in combination with one or more additional active agents.

Lipid-Based Particles are expected to be useful when used with: alkylating agents, angiogenesis inhibitors, antibodies, antimetabolites, antimitotics, antiproliferatives, aurora kinase inhibitors, apoptosis promoters (for example, Bcl-xL, Bcl-w and Bfl-l) inhibitors, Bcr-Abl kinase inhibitors, BiTE (Bi-Specific T cell Engager) antibodies, biologic response modifiers, cyclin-dependent kinase inhibitors, cell cycle inhibitors, cyclooxygenase-2 inhibitors, DVD's, leukemia viral oncogene homolog (ErbB2) receptor inhibitors, growth factor inhibitors, heat shock protein (HSP)-90 inhibitors, histone deacetylase (HDAC) inhibitors, hormonal therapies, immunologicals, inhibitors of apoptosis proteins (IAP's) intercalating antibiotics, kinase inhibitors, mammalian target of rapamycin inhibitors, microRNA's mitogen-activated extracellular signal-regulated kinase inhibitors, multivalent binding proteins, non-steroidal anti-inflammatory drugs (NSAIDs), poly ADP (adenosine diphosphate)-ribose polymerase (PARP) inhibitors, platinum chemotherapeutics, polo-like kinase (Plk) inhibitors, proteosome inhibitors, purine analogs, pyrimidine analogs, receptor tyrosine kinase inhibitors, retinoids/deltoids plant alkaloids, small inhibitory ribonucleic acids (siRNA's), topoisomerase inhibitors, combinations thereof and the like.

The pharmaceutical composition and the method of the present invention may further comprise other therapeutically active compounds as noted herein which are usually applied in the treatment of the pathological conditions.

A BiTE antibody is a bi-specific antibody that directs T-cells to attach cancer cells by simultaneously binding the two cells. The T-cell then attacks the target cancer cell. Exemplary BiTE antibodies include adecatumumab (Micromet MT201), blinatumomab (Micromet MT103) and the like.

SiRNA's are molecules having endogenous RNA bases or chemically modified nucleotides. The modifications shall not abolish cellular activity, but rather impart increased stability and/or increased cellular potency. Examples of chemical modifications include phosphorothioate groups, 2′-deoxynucleotide, 2′-OCH₃-containing ribonucleotides, 2′-F-ribonucleotides, 2′-methoxyethyl ribonucleotides or a combination thereof. The siRNA can have varying lengths (10-200 bps) and structures (hairpins, single/double strands, bulges, nicks/gaps, mismatches) and processed in the cell to provide active gene silencing. In certain embodiments, a double-stranded siRNA (dsRNA) can have the same number of nucleotides on each strand (blunt ends) or asymmetric ends (overhangs). The overhang of 1-2 nucleotides can be present on the sense and/or the antisense strand, as well as present on the 5′- and/or the 3′-ends of a given strand.

Multivalent binding proteins are binding proteins comprising two or more antigen binding sites. The multivalent binding protein is preferably engineered to have the three or more antigen binding sites and is generally not a naturally occurring antibody. The term “multispecific binding protein” means a binding protein capable of binding two or more related or unrelated targets. Dual variable domain (DVD) binding proteins are tetravalent or multivalent binding proteins binding proteins comprising two or more antigen binding sites. Such DVDs may be monospecific, i.e., capable of binding one antigen or multispecific, i.e., capable of binding two or more antigens. DVD binding proteins comprising two heavy chain DVD polypeptides and two light chain DVD polypeptides are referred to as DVD Ig. Each half of a DVD Ig comprises a heavy chain DVD polypeptide, a light chain DVD polypeptide, and two antigen binding sites. Each binding site comprises a heavy chain variable domain and a light chain variable domain with a total of 6 CDRs involved in antigen binding per antigen binding site.

Alkylating agents include altretamine, AMD-473, AP-5280, apaziquone, bendamustine, brostallicin, busulfan, carboquone, carmustine (BCNU), chlorambucil, CLORETAZINE® (laromustine, VNP 40101M), cyclophosphamide, decarbazine, estramustine, fotemustine, glufosfamide, ifosfamide, KW-2170, lomustine (CCNU), mafosfamide, melphalan, mitobronitol, mitolactol, nimustine, nitrogen mustard N-oxide, ranimustine, temozolomide, thiotepa, TREANDA® (bendamustine), treosulfan, rofosfamide and the like.

Angiogenesis inhibitors include endothelial-specific receptor tyrosine kinase (Tie-2) inhibitors, epidermal growth factor receptor (EGFR) inhibitors, insulin growth factor-2 receptor (IGFR-2) inhibitors, matrix metalloproteinase-2 (MMP-2) inhibitors, matrix metalloproteinase-9 (MMP-9) inhibitors, platelet-derived growth factor receptor (PDGFR) inhibitors, thrombospondin analogs, vascular endothelial growth factor receptor tyrosine kinase (VEGFR) inhibitors and the like.

Antimetabolites include ALIMTA® (metrexed disodium, LY231514, MTA), 5-azacitidine, XELODA® (capecitabine), carmofur, LEUSTAT® (cladribine), clofarabine, cytarabine, cytarabine ocfosfate, cytosine arabinoside, decitabine, deferoxamine, doxifluridine, eflornithine, EICAR (5-ethynyl-1-β-D-ribofuranosylimidazole-4-carboxamide), enocitabine, ethnylcytidine, fludarabine, 5-fluorouracil alone or in combination with leucovorin, GEMZAR® (gemcitabine), hydroxyurea, ALKERAN® (melphalan), mercaptopurine, 6-mercaptopurine riboside, methotrexate, mycophenolic acid, nelarabine, nolatrexed, ocfosfate, pelitrexol, pentostatin, raltitrexed, Ribavirin, triapine, trimetrexate, S-1, tiazofurin, tegafur, TS-1, vidarabine, UFT and the like.

Bcl-2 proteins inhibitors include AT-101 ((−)gossypol), GENASENSE® (G3139 or oblimersen (Bcl-2-targeting antisense oligonucleotide)), IPI-194, IPI-565, N-(4-(4-((4′-chloro(1,1′-biphenyl)-2-yl)methyl)piperazin-1-yl)benzoyl)-4-(((1R)-3-(dimethylamino)-1-((phenylsulfanyl)methyl)propyl)amino)-3-nitrobenzenesulfonamide) (ABT-737), N-(4-(4-((2-(4-chlorophenyl)-5,5-dimethyl-1-cyclohex-1-en-1-yl)methyl)piperazin-1-yl)benzoyl)-4-(((1R)-3-(morpholin-4-yl)-1-((phenylsulfanyl)methyl)propyl)amino)-3-((trifluoromethyl)sulfonyl)benzenesulfonamide (ABT-263), GX-070 (obatoclax) and the like.

Bcr-Abl kinase inhibitors include DASATINIB® (BMS-354825), GLEEVEC® (imatinib) and the like.

CDK inhibitors include AZD-5438, BMI-1040, BMS-032, BMS-387, CVT-2584,

flavopyridol, GPC-286199, MCS-5A, PD0332991, PHA-690509, seliciclib (CYC-202, R-roscovitine), ZK-304709 and the like.

COX-2 inhibitors include ABT-963, ARCOXIA® (etoricoxib), BEXTRA® (valdecoxib), BMS347070, CELEBREX® (celecoxib), COX-189 (lumiracoxib), CT-3, DERAMAXX® (deracoxib), JTE-522,4-methyl-2-(3,4-dimethylphenyl)-1-(4-sulfamoylphenyl-1H-pyrrole), MK-663 (etoricoxib), NS-398, parecoxib, RS-57067, SC-58125, SD-8381, SVT-2016, S-2474, T-614, VIOXX® (rofecoxib) and the like.

EGFR inhibitors include ABX-EGF, anti-EGFR immunoliposomes, EGF-vaccine, EMD-7200, ERBITUX® (cetuximab), HR3, IgA antibodies, IRESSA® (gefitinib), TARCEVA® (erlotinib or OSI-774), TP-38, EGFR fusion protein, TYKERB® (lapatinib) and the like.

ErbB2 receptor inhibitors include CP-724-714, CI-1033 (canertinib), HERCEPTIN® (trastuzumab), TYKERB® (lapatinib), OMNITARG® (2C4, petuzumab), TAK-165, GW-572016 (ionafarnib), GW-282974, EKB-569, PI-166, dHER2 (HER2 vaccine), APC-8024 (HER-2 vaccine), anti-HER/2neu bispecific antibody, B7.her2IgG3, AS HER2 trifunctional bispecfic antibodies, mAB AR-209, mAB 2B-1 and the like.

Histone deacetylase inhibitors include depsipeptide, LAQ-824, MS-275, trapoxin, suberoylanilide hydroxamic acid (SAHA), TSA, valproic acid and the like.

HSP-90 inhibitors include 17-AAG-nab, 17-AAG, CNF-101, CNF-1010, CNF-2024, 17-DMAG, geldanamycin, IPI-504, KOS-953, MYCOGRAB® (human recombinant antibody to HSP-90), NCS-683664, PU24FC1, PU-3, radicicol, SNX-2112, STA-9090 VER49009 and the like.

Inhibitors of apoptosis proteins include ApoMab (a fully human affinity-matured IgG1 monoclonal antibody), antibodies that target TRAIL or death receptors (e.g., pro-apoptotic receptor agonists DR4 and DR5), conatumumab, ETR2-ST01, GDC0145, (lexatumumab), HGS-1029, LBY-135, PRO-1762 and tratuzumab.

MEK inhibitors include ARRY-142886, ARRY-438162 PD-325901, PD-98059 and the like.

mTOR inhibitors include AP-23573, CCI-779, everolimus, RAD-001, rapamycin, temsirolimus and the like.

Non-steroidal anti-inflammatory drugs include AMIGESIC® (salsalate), DOLOBID® (diflunisal), MOTRIN® (ibuprofen), ORUDIS® (ketoprofen), RELAFEN® (nabumetone), FELDENE® (piroxicam), ibuprofen cream, ALEVE® (naproxen) and NAPROSYN® (naproxen), VOLTAREN® (diclofenac), INDOCIN® (indomethacin), CLINORIL® (sulindac), TOLECTIN® (tolmetin), LODINE® (etodolac), TORADOL® (ketorolac), DAYPRO® (oxaprozin) and the like.

PDGFR inhibitors include C-451, CP-673, CP-868596 and the like.

Platinum chemotherapeutics include cisplatin, ELOXATIN® (oxaliplatin) eptaplatin, lobaplatin, nedaplatin, PARAPLATIN® (carboplatin), satraplatin and the like.

Polo-like kinase inhibitors include BI-2536 and the like.

Thrombospondin analogs include ABT-510, ABT-567, TSP-1 and the like.

VEGFR inhibitors include AVASTIN® (bevacizumab), ABT-869, AEE-788, ANGIOZYME™ (a ribozyme that inhibits angiogenesis (Ribozyme Pharmaceuticals (Boulder, Colo.) and Chiron, (Emeryville, Calif.)), axitinib (AG-13736), AZD-2171, CP-547,632, IM-862, MACUGEN (pegaptamib), NEXAVAR® (sorafenib, BAY43-9006), pazopanib (GW-786034), vatalanib (PTK-787, ZK-222584), SUTENT® (sunitinib, SU-11248), VEGF trap, ZACTIMA™ (vandetanib, ZD-6474) and the like.

Antibiotics include intercalating antibiotics aclarubicin, actinomycin D, amrubicin, annamycin, adriamycin, BLENOXANE® (bleomycin), daunorubicin, CAELYX® or MYOCET® (liposomal doxorubicin), elsamitrucin, epirbucin, glarbuicin, ZAVEDOS® (idarubicin), mitomycin C, nemorubicin, neocarzinostatin, peplomycin, pirarubicin, rebeccamycin, stimalamer, streptozocin, VALSTAR® (valrubicin), zinostatin and the like.

Topoisomerase inhibitors include aclarubicin, 9-aminocamptothecin, amonafide, amsacrine, becatecarin, belotecan, BN-80915, CAMPTOSAR® (irinotecan hydrochloride), camptothecin, CARDIOXANE® (dexrazoxine), diflomotecan, edotecarin, ELLENCE® or PHARMORUBICIN® (epirubicin), etoposide, exatecan, 10-hydroxycamptothecin, gimatecan, lurtotecan, mitoxantrone, orathecin, pirarbucin, pixantrone, rubitecan, sobuzoxane, SN-38, tafluposide, topotecan and the like.

Antibodies include AVASTIN® (bevacizumab), CD40-specific antibodies, chTNT-1/B, denosumab, ERBITUX® (cetuximab), HUMAX-CD4® (zanolimumab), IGF1R-specific antibodies, lintuzumab, PANOREX® (edrecolomab), RENCAREX® (WX G250), RITUXAN® (rituximab), ticilimumab, trastuzimab and the like.

Hormonal therapies include ARIMIDEX® (anastrozole), AROMASIN® (exemestane), arzoxifene, CASODEX® (bicalutamide), CETROTIDE® (cetrorelix), degarelix, deslorelin, DESOPAN® (trilostane), dexamethasone, DROGENIL®, (flutamide), EVISTA® (raloxifene), AFEMA™ (fadrozole), FARESTON® (toremifene), FASLODEX® (fulvestrant), FEMARA® (letrozole), formestane, glucocorticoids, HECTOROL® (doxercalciferol), RENAGEL® (sevelamer carbonate), lasofoxifene, leuprolide acetate, MEGACE® (megesterol), MIFEPREX® (mifepristone), NILANDRON™ (nilutamide), NOLVADEX® (tamoxifen citrate), PLENAXIS™ (abarelix), prednisone, PROPECIA® (finasteride), rilostane, SUPREFACT® (buserelin), TRELSTAR® (luteinizing hormone releasing hormone (LHRH)), VANTAS® (Histrelin implant), VETORYL® (trilostane or modrastane), ZOLADEX® (fosrelin, goserelin) and the like.

Deltoids and retinoids include seocalcitol (EB1089, CB1093), lexacalcitrol (KH1060), fenretinide, PANRETIN® (aliretinoin), ATRAGEN® (liposomal tretinoin), TARGRETIN® (bexarotene), LGD-1550 and the like.

PARP inhibitors include ABT-888, olaparib, KU-59436, AZD-2281, AG-014699, BSI-201, BGP-15, INO-1001, ONO-2231 and the like.

Plant alkaloids include, but are not limited to, vincristine, vinblastine, vindesine, vinorelbine and the like.

Proteasome inhibitors include VELCADE® (bortezomib), MG132, NPI-0052, PR-171 and the like.

Examples of immunologicals include interferons and other immune-enhancing agents. Interferons include interferon alpha, interferon alpha-2a, interferon alpha-2b, interferon beta, interferon gamma-1a, ACTIMMUNE® (interferon gamma-1b), or interferon gamma-n1, combinations thereof and the like. Other agents include ALFAFERONE®, (IFN-α), BAM-002 (oxidized glutathione), BEROMUN® (tasonermin), BEXXAR® (tositumomab), CAMPATH® (alemtuzumab), CTLA4 (cytotoxic lymphocyte antigen 4), decarbazine, denileukin, epratuzumab, GRANOCYTE® (lenograstim), lentinan, leukocyte alpha interferon, imiquimod, MDX-010 (anti-CTLA-4), melanoma vaccine, mitumomab, molgramostim, MYLOTARG™ (gemtuzumab ozogamicin), NEUPOGEN® (filgrastim), OncoVAC-CL, OVAREX® (oregovomab), pemtumomab (Y-muHMFG1), PROVENGE® (sipuleucel-T), sargaramostim, sizofilan, teceleukin, THERACYS® (Bacillus Calmette-Guerin), ubenimex, VIRULIZIN® (immunotherapeutic, Lorus Pharmaceuticals), Z-100 (Specific Substance of Maruyama (SSM)), WF-10 (Tetrachlorodecaoxide (TCDO)), PROLEUKIN® (aldesleukin), ZADAXIN® (thymalfasin), ZENAPAX® (daclizumab), ZEVALIN® (90Y-Ibritumomab tiuxetan) and the like.

Biological response modifiers are agents that modify defense mechanisms of living organisms or biological responses, such as survival, growth, or differentiation of tissue cells to direct them to have anti-tumor activity and include include krestin, lentinan, sizofuran, picibanil PF-3512676 (CpG-8954), ubenimex and the like.

Pyrimidine analogs include cytarabine (ara C or Arabinoside C), cytosine arabinoside, doxifluridine, FLUDARA® (fludarabine), 5-FU (5-fluorouracil), floxuridine, GEMZAR® (gemcitabine), TOMUDEX® (ratitrexed), TROXATYL™ (triacetyluridine troxacitabine) and the like.

Purine analogs include LANVIS® (thioguanine) and PURI-NETHOL® (mercaptopurine).

Antimitotic agents include batabulin, epothilone D (KOS-862), N-(2-((4-hydroxyphenyl)amino)pyridin-3-yl)-4-methoxybenzenesulfonamide, ixabepilone (BMS 247550), paclitaxel, TAXOTERE® (docetaxel), PNU100940 (109881), patupilone, XRP-9881 (larotaxel), vinflunine, ZK-EPO (synthetic epothilone) and the like.

Compounds of this invention can also be used as radiosensitizeser that enhance the efficacy of radiotherapy. Examples of radiotherapy include external beam radiotherapy, teletherapy, brachtherapy and sealed, unsealed source radiotherapy and the like.

Additionally, compounds having Formula I may be combined with other chemptherapeutic agents such as ABRAXANE™ (ABI-007), ABT-100 (farnesyl transferase inhibitor), ADVEXIN® (Ad5CMV-p53 vaccine), ALTOCOR® or MEVACOR® (lovastatin), AMPLIGEN® (poly I:poly C12U, a synthetic RNA), APTOSYN® (exisulind), AREDIA® (pamidronic acid), arglabin, L-asparaginase, atamestane (1-methyl-3,17-dione-androsta-1,4-diene), AVAGE® (tazarotene), AVE-8062 (combreastatin derivative) BEC2 (mitumomab), cachectin or cachexin (tumor necrosis factor), canvaxin (vaccine), CEAVAC® (cancer vaccine), CELEUK® (celmoleukin), CEPLENE® (histamine dihydrochloride), CERVARIX® (human papillomavirus vaccine), CHOP® (C: CYTOXAN® (cyclophosphamide); H: ADRIAMYCIN® (hydroxydoxorubicin); 0: Vincristine (ONCOVIN®); P: prednisone), CYPAT™ (cyproterone acetate), combrestatin A4P, DAB(389)EGF (catalytic and translocation domains of diphtheria toxin fused via a His-Ala linker to human epidermal growth factor) or TransMID-107R™ (diphtheria toxins), dacarbazine, dactinomycin, 5,6-dimethylxanthenone-4-acetic acid (DMXAA), eniluracil, EVIZON™ (squalamine lactate), DIMERICINE® (T4N5 liposome lotion), discodermolide, DX-8951f (exatecan mesylate), enzastaurin, EP0906 (epithilone B), GARDASIL® (quadrivalent human papillomavirus (Types 6, 11, 16, 18) recombinant vaccine), GASTRIMMUNE®, GENASENSE®, GMK (ganglioside conjugate vaccine), GVAX® (prostate cancer vaccine), halofuginone, histerelin, hydroxycarbamide, ibandronic acid, IGN-101, IL-13-PE38, IL-13-PE38QQR (cintredekin besudotox), IL-13-pseudomonas exotoxin, interferon-α, interferon-γ, JUNOVAN™ or MEPACT™ (mifamurtide), lonafarnib, 5,10-methylenetetrahydrofolate, miltefosine (hexadecylphosphocholine), NEOVASTAT® (AE-941), NEUTREXIN® (trimetrexate glucuronate), NIPENT® (pentostatin), ONCONASE® (a ribonuclease enzyme), ONCOPHAGE® (melanoma vaccine treatment), ONCOVAX® (IL-2 Vaccine), ORATHECINT™ (rubitecan), OSIDEM® (antibody-based cell drug), OVAREX® MAb (murine monoclonal antibody), paditaxel, PANDIMEX™ (aglycone saponins from ginseng comprising 20(S)protopanaxadiol (aPPD) and 20(S)protopanaxatriol (aPPT)), panitumumab, PANVAC®-VF (investigational cancer vaccine), pegaspargase, PEG Interferon A, phenoxodiol, procarbazine, rebimastat, REMOVAB® (catumaxomab), REVLIMID® (lenalidomide), RSR13 (efaproxiral), SOMATULINE® LA (lanreotide), SORIATANE® (acitretin), staurosporine (Streptomyces staurospores), talabostat (PT100), TARGRETIN® (bexarotene), TAXOPREXIN® (DHA-paclitaxel), TELCYTA® (canfosfamide, TLK286), temilifene, TEMODAR® (temozolomide), tesmilifene, thalidomide, THERATOPE® (STn-KLH), thymitaq (2-amino-3,4-dihydro-6-methyl-4-oxo-5-(4-pyridylthio)quinazoline dihydrochloride), TNFERADE™ (adenovector: DNA carrier containing the gene for tumor necrosis factor-α), TRACLEER® or ZAVESCA® (bosentan), tretinoin (Retin-A), tetrandrine, TRISENOX® (arsenic trioxide), VIRULIZIN®, ukrain (derivative of alkaloids from the greater celandine plant), vitaxin (anti-alphavbeta3 antibody), XCYTRIN® (motexafin gadolinium), XINLAY™ (atrasentan), XYOTAX™ (paclitaxel poliglumex), YONDELIS® (trabectedin), ZD-6126, ZINECARD® (dexrazoxane), ZOMETA® (zolendronic acid), zorubicin and the like.

### Cationic-Based Lipid Encapsulation Systems (CaBLES) and Lipid-Based Particles

CaBLES comprise one or more non-cationic lipids, one or more cationic lipids and one or more polyethylene glycol (PEG)-lipid conjugates having Formula I.

Lipid-Based Particles of the present invention are defined as CaBLES which further comprise one or more therapeutic agent(s). These particles have mean diameter sizes of 50-300 nm, of which 50-250 nm is preferred and 50-200 nm is most preferred. Functional CaBLES effectively encapsulate nucleic acids, (e.g., single stranded or double stranded DNA, single stranded or double stranded RNA, RNAi, siRNA, and the like). Suitable nucleic acids include, but are not limited to, plasmids, antisense oligonucleotides, ribozymes as well as other poly- and oligonucleotides. In preferred embodiments, the nucleic acid encodes a product, e.g., a therapeutic product, of interest. The CaBLES of the present invention can be used to deliver the nucleic acid to a cell (e.g., a cell in a mammal) for, e.g., expression of the nucleic acid or for silencing of a target sequence expressed by the cell.

In some embodiments, the nucleic acid is a siRNA molecule that silences the gene of interest, with efficiencies from about 50-100%, and more preferably between about 80-100%.

In other embodiments, the therapeutic agents that can be delivered with CaBLES include RNA, antisense oligonucleotide, a DNA, a plasmid, a ribosomal RNA (rRNA), a micro RNA (miRNA), transfer RNA (tRNA), a small inhibitory RNA (siRNA), small nuclear RNA (snRNA), chimeric nucleic acids, an antigen, fragments thereof, a protein, a peptide, small-molecules, or mixtures thereof. This invention describes delivery of RNA's such as small inhibitory RNA or microRNA. The siRNA can have varying lengths (10-200 bps) and structures (hairpins, single/double strands, bulges, nicks/gaps, mismatches) and processed in the cell to provide active gene silencing. In certain embodiments of this invention, a double-stranded siRNA (dsRNA) can have the same number of nucleotides on each strand (blunt ends) or asymmetric ends (overhangs). The overhang of 1-2 nucleotides can be present on the sense and/or the antisense strand, as well as present on the 5′- and/or the 3′-ends of a given strand.

Suitable siRNA sequences can be identified using means known in the art (e.g., methods described in Elbashir, et al., Nature 411:494-498 (2001) and Elbashir, et al., EMBO J. 20: 6877-6888 (2001) are combined with rational design rules set forth in Reynolds et al., Nature Biotech. 22(3):326-330 (2004)). Further enhancing, isolating, synthesizing and generating of the siRNA can be done by various methods known in the art, (see, e.g., Elbashir, et al., EMBO J. 20: 6877-6888 (2001); Elbashir, et al., Genes Dev. 15:188 (2001); Nykanen, et al., Cell 107:309 (2001)) or may lack overhangs (i.e., to have blunt ends): and Gubler & Hoffman, Gene 25:263-269 (1983); Sambrook et al., Molecular Cloning, A Laboratory Manual (2nd ed. 1989); Current Protocols in Molecular Biology (Ausubel et al., eds., 1994), as are PCR methods (see U.S. Pat. Nos. 4,683,195 and 4,683,202; PCR Protocols: A Guide to Methods and Applications (Innis et al., eds, 1990)).

Non-cationic lipids have a neutral charge or an anionic charge at physiological pH. A neutral lipid, also known as a “helper lipid,” has no net charge at physiological pH. These lipids can also be zwitterionic.

Polyethylene glycol (PEG)-lipid conjugates are used to minimize particle aggregation in solution, provide increased in vivo serum circulation, and enhance distribution of nanoparticles to organs, tissues, cell types, and tumors of interest. These shielding lipids consist of a lipid portion linked to a “PEG” portion via carbamate, ester, amide, ether, amine, thioether, or dithiol linkages. “PEG” is a polyethylene glycol consisting of repeating C₂H₄O units with an average molecular weight between 500 to 10,000 daltons and may be substituted by alkoxy, acyl, alkyl, or aryl. Additionally, the PEG can be substituted at its terminus with one or more of the following functional groups: hydroxy, methoxy, primary, secondary, or tertiary amine, thiol, thioether, thiopyridyl, dithiol, maleimide, or ester. Particular polyethylene glycol (PEG)-lipid conjugates of this invention are as described in Formula I and include 6-oxo-2-(tetradecanoyloxy)-8,11,14,17,20,23,26,29,32,35,38,41,44,47,50,53,56,59,62,65,68,71,74,77,80,83,86,89,92, 95,98,101,104,107,110,113,116,119,122,125,128,131,134,137,140,143-hexatetracontaoxa-5-azatetratetracontahect-1-yl myristate; N-[3,4-bis(tetradecyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide; N-[3,4-bis(hexadecyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide; N-[3,4-bis(octadecyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide; 3,7,11,15,19,23,27,31,35,39,43,47,51,55,59,63,67,71,75,79,83,87,91,95,99,103,107,111,115, 119,123,127,131,135,139,143,147,151,155,159,163,167,171,175,179,182-hexatetracontaoxatrioctacontahect-1-yl 3,4-bis(tetradecyloxy)butylcarbamate; 3,6,9,12,15,18,21,24,27,30,33,36,39,42,45,48,51,54,57,60,63,66,69,72,75,78,81,84,87,90, 93,96,99,102,105,108,111,114,117,120,123,126,129,132,135,138-hexatetracontaoxanonatriacontahect-1-yl 3,4-bis(hexadecyloxy)butylcarbamate; 3,6,9,12,15,18,21,24,27,30,33,36,39,42,45,48,51,54,57,60,63,66,69,72,75,78,81,84,87,90, 93,96,99,102,105,108,111,114,117,120,123,126,129,132,135,138-hexatetracontaoxanonatriacontahect-1-yl 3,4-bis(octadecyloxy)butylcarbamate; N-[3,4-bis(hexadecyloxy)butyl]-N′-3,6,9,12,15,18,21,24,27,30,33,36,39,42,45,48,51,54,57,60,63,66,69,72,75,78,81,84,87,90, 93,96,99,102,105,108,111,114,117,120,123,126,129,132,135,138-hexatetracontaoxanonatriacontahect-1-ylsuccinamide; 6-oxo-2-(tetradecanoyloxy)-7,10,13,16,19,22,25,28,31,34,37,40,43,46,49,52,55,58,61,64,67,70,73,76,79,82,85,88,91, 94,97,100,103,106,109,112,115,118,121,124,127,130,133,136,139,142,145-heptatetracontaoxa-5-azahexatetracontahect-1-yl myristate; 6-oxo-2-(palmitoyloxy)-7,10,13,16,19,22,25,28,31,34,37,40,43,46,49,52,55,58,61,64,67,70,73,76,79,82,85,88,91, 94,97,100,103,106,109,112,115,118,121,124,127,130,133,136,139,142,145-heptatetracontaoxa-5-azahexatetracontahect-1-yl palmitate; 3,6,9,12,15,18,21,24,27,30,33,36,39,42,45,48,51,54,57,60,63,66,69,72,75,78,81,84,87,90, 93,96,99,102,105,108,111,114,117,120,123,126,129,132,135,138-hexatetracontaoxanonatriacontahect-1-yl 4-{[3,4-bis(hexadecyloxy)butyl]amino}-4-oxobutanoate; 6-oxo-2-(palmitoyloxy)-8,11,14,17,20,23,26,29,32,35,38,41,44,47,50,53,56,59,62,65,68,71,74,77,80,83,86,89,92, 95,98,101,104,107,110,113,116,119,122,125,128,131,134,137,140,143-hexatetracontaoxa-5-azatetratetracontahect-1-yl palmitate; N-[4-(decyloxy)-3-(octadecyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide; N-[3,4-bis(decyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide; N-[3-(octadecyloxy)-4-(tetradecyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide; N-[4-(hexadecyloxy)-3-(octadecyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide; N-[3,4-bis(hexadecyloxy)butyl]-2,5,8,11,14,17,20,23,26,29,32,35,38,41,44,47,50,53,56,59,62,65,68-tricosaoxaheptacontan-70-amide; N-[3,4-bis(hexadecyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137,140,143,146,149,152,155, 158,161,164,167,170,173,176,179,182,185,188,191,194,197,200,203,206,209,212,215, 218,221,224,227,230,233,236,239,242,245,248,251,254,257,260,263,266,269,272,275,278, 281,284,287,290,293,296,299,302,305,308,311,314,317,320,323,326,329,332,335,338-113oxa340n-340-amide; and N-[3-(hexadecyloxy)-4-(octadecyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide. Polyethylene glycol (PEG)-lipid conjugates that are useful for the practice of this invention include, but are not limited to 6-oxo-2-(tetradecanoyloxy)-8,11,14,17,20,23,26,29,32,35,38,41,44,47,50,53,56,59,62,65,68,71,74,77,80,83,86,89,92, 95,98,101,104,107,110,113,116,119,122,125,128,131,134,137,140,143-hexatetracontaoxa-5-azatetratetracontahect-1-yl myristate; N-[3,4-bis(tetradecyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide; N-[3,4-bis(hexadecyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide; N-[3,4-bis(octadecyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide; 3,7,11,15,19,23,27,31,35,39,43,47,51,55,59,63,67,71,75,79,83,87,91,95,99,103,107,111,115, 119,123,127,131,135,139,143,147,151,155,159,163,167,171,175,179,182-hexatetracontaoxatrioctacontahect-1-yl 3,4-bis(tetradecyloxy)butylcarbamate; 3,6,9,12,15,18,21,24,27,30,33,36,39,42,45,48,51,54,57,60,63,66,69,72,75,78,81,84,87,90, 93,96,99,102,105,108,111,114,117,120,123,126,129,132,135,138-hexatetracontaoxanonatriacontahect-1-yl 3,4-bis(hexadecyloxy)butylcarbamate; 3,6,9,12,15,18,21,24,27,30,33,36,39,42,45,48,51,54,57,60,63,66,69,72,75,78,81,84,87,90, 93,96,99,102,105,108,111,114,117,120,123,126,129,132,135,138-hexatetracontaoxanonatriacontahect-1-yl 3,4-bis(octadecyloxy)butylcarbamate; N-[3,4-bis(hexadecyloxy)butyl]-N′-3,6,9,12,15,18,21,24,27,30,33,36,39,42,45,48,51,54,57,60,63,66,69,72,75,78,81,84,87,90, 93,96,99,102,105,108,111,114,117,120,123,126,129,132,135,138-hexatetracontaoxanonatriacontahect-1-ylsuccinamide; 6-oxo-2-(tetradecanoyloxy)-7,10,13,16,19,22,25,28,31,34,37,40,43,46,49,52,55,58,61,64,67,70,73,76,79,82,85,88,91, 94,97,100,103,106,109,112,115,118,121,124,127,130,133,136,139,142,145-heptatetracontaoxa-5-azahexatetracontahect-1-yl myristate; 6-oxo-2-(palmitoyloxy)-7,10,13,16,19,22,25,28,31,34,37,40,43,46,49,52,55,58,61,64,67,70,73,76,79,82,85,88,91, 94,97,100,103,106,109,112,115,118,121,124,127,130,133,136,139,142,145-heptatetracontaoxa-5-azahexatetracontahect-1-yl palmitate; 3,6,9,12,15,18,21,24,27,30,33,36,39,42,45,48,51,54,57,60,63,66,69,72,75,78,81,84,87,90, 93,96,99,102,105,108,111,114,117,120,123,126,129,132,135,138-hexatetracontaoxanonatriacontahect-1-yl 4-{[3,4-bis(hexadecyloxy)butyl]amino}-4-oxobutanoate; 6-oxo-2-(palmitoyloxy)-8,11,14,17,20,23,26,29,32,35,38,41,44,47,50,53,56,59,62,65,68,71,74,77,80,83,86,89,92, 95,98,101,104,107,110,113,116,119,122,125,128,131,134,137,140,143-hexatetracontaoxa-5-azatetratetracontahect-1-yl palmitate; N-[4-(decyloxy)-3-(octadecyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide; N-[3,4-bis(decyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide; N-[3-(octadecyloxy)-4-(tetradecyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide; N-[4-(hexadecyloxy)-3-(octadecyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide; N-[3,4-bis(hexadecyloxy)butyl]-2,5,8,11,14,17,20,23,26,29,32,35,38,41,44,47,50,53,56,59,62,65,68-tricosaoxaheptacontan-70-amide; N-[3,4-bis(hexadecyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137,140,143,146,149,152,155, 158,161,164,167,170,173,176,179,182,185,188,191,194,197,200,203,206,209,212,215, 218,221,224,227,230,233,236,239,242,245,248,251,254,257,260,263,266,269,272,275,278, 281,284,287,290,293,296,299,302,305,308,311,314,317,320,323,326,329,332,335,338-113oxa340n-340-amide, N-[3-(hexadecyloxy)-4-(octadecyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide, 1,2-distearoyl-sn-glycerol-methoxypolyethyleneglycol-750,1,2-dimyristoyl-sn-glycerol-methoxypolyethyleneglycol-750,1,2-dipalmitoyl-sn-glycerol-methoxypolyethyleneglycol-750, poly(oxy-1,2-ethanediyl)-2000-α-(313)-cholest-5-en-3-yl-omega-hydroxy, 1,2-dipalmitoyl-sn-glycerol-methoxypolyethyleneglycol-5000, poly(oxy-1,2-ethanediyl)-5000-α-(3β)-cholest-5-en-3-yl-omega-hydroxy, (2S,3R,E)-3-hydroxy-2-stearamidooctadec-4-enyl polyethyleneglycol-2000 methyl ether succinate, (2S,3R,E)-3-hydroxy-2-icosanamidooctadec-4-enyl polyethyleneglycol-2000 methyl ether succinate, N-(2,3-dimyristyloxypropyl)carbamate polyethyleneglycol-2000 methyl ether, N-(carbonylmethoxypolyethyleneglycol-750)-1,2-dimyristoyl-sn-glycero-phosphatidylethanolamine, N-(carbonyl-methoxypolyethyleneglycol-750)-1,2-distearoyl-sn-glycero-3-phosphoethanolamine, N-(carbonyl-methoxypolyethyleneglycol-750)-1,2-dipalmitoyl-sn-glycero-3-phosphoethanolamine, N-(carbonyl-methoxypolyethyleneglycol-2000)-1,2-dimyristoyl-sn-glycero-3-phosphoethanolamine, N-(carbonyl-methoxypolyethyleneglycol-2000)-1,2-dipalmitoyl-sn-glycero-3-phosphoethanolamine, N-(carbonyl-methoxypolyethyleneglycol-2000)-1,2-distearoyl-sn-glycero-3-phosphoethanolamine, N-(carbonyl-methoxypolyethyleneglycol-2000)-dioleoyl-phosphatidylethanolamine, 1,2-distearoyl-sn-glycerol-methoxypolyethyleneglycol-2000,1,2-dimyristoyl-sn-glycerol-methoxypolyethyleneglycol-2000,1,2-dipalmitoyl-sn-glycerol-methoxypolyethyleneglycol-2000, mPEG-2000-cholesterol, octanoyl-mPEG-2000-ceramide, palmitoyl-mPEG-2000-ceramide, N-(carbonyl-methoxypolyethyleneglycol-5000)-1,2-dimyristoyl-sn-glycero-3-phosphoethanolamine, N-(carbonyl-methoxypolyethyleneglycol-5000)-1,2-dipalmitoyl-sn-glycero-3-phosphoethanolamine, N-(carbonyl-methoxypolyethyleneglycol-5000)-1,2-distearoyl-sn-glycero-3-phosphoethanolamine, 1,2-dimyristoyl-sn-glycerol-methoxypolyethyleneglycol-5000, 1,2-dipalmitoyl-sn-glycerol-methoxypolyethyleneglycol-5000,1,2-distearoyl-sn-glycerol-methoxypolyethyleneglycol-5000, mPEG-5000-cholesterol, octanoyl-mPEG-5000-ceramide, palmitoyl-mPEG-5000-ceramide and mixtures thereof.

In some instances it may be desirable for the CaBLES and/or Lipid Based Particles to target using targeting moieties that are specific to a cell type or tissue. Targeting of liposomes using a variety of targeting moieties, such as ligands, cell surface receptors, glycoproteins, vitamins, (e.g., ribolflavin) and moncoleonal antibodies, has been previously described (see, e.g., U.S. Pat. Nos. 4,957,773 and 4,603,044). The targeting moeities can comprise the entire protein or fragments thereof. In one aspect, the targeting moiety is a small protein, or peptide. In another aspect, the targeting moiety is a small-molecule.

Cationic lipids are those having one or more moieties that are positively charged at a physiologically relevant pH, typically between 4-8. Examples of cationic lipids that are useful for the practice of this invention include, but are not limited to, N,N-dioleyl-N,N-dimethylammonium chloride, DC-Chol; 1,3-dioleoyloxy-2-(6-carboxyspermyl)-propyl amide, dioctadecylamidoglycyl spermine, N,N-distearyl-N,N-dimethylammonium bromide, N-(2,3-dioleyloxy)propyl)-N,N-dimethylammonium chloride, 1,2-dioleoyl-3-trimethylammonium-propane chloride, 1,2-dilineoyl-3-dimethylammonium-propane, N-(1-(2,3-dioleyloxy)propyl)-N,N,N-trimethylammonium chloride, 1,2-dioleoyl-3-dimethylammonium propane, 1,2-distearyloxy-N,N-dimethyl-3-aminopropane; didodecyldimethylammonium bromide, dioleoyloxy-N-(2-sperminecarboxamido)ethyl)-N,N-dimethyl-1-propanaminiumtrifluoroacetate, 1,2-dimyristyloxypropyl-3-dimethyl-hydroxyethyl ammonium bromide, 1,2-dioleoylcarbamyl-3-dimethylammoniumpropane, tetramethyltetrapalmitoyl spermine, tetramethyltetraoleyl spermine, tetramethyldioleyl spermine, tetramethyltetramyristyl spermine, tetramethyltetralauryl spermine, 1-(2-((9Z,12Z)-octadeca-9,12-dienyloxy)-1-(((9Z,12Z)-octadeca-9,12-dienyloxy)methyl)ethyl)pyrrolidine; N,N-dimethyl-N-(2-((9Z,12Z)-octadeca-9,12-dienyloxy)-1-(((9Z,12Z)-octadeca-9,12-dienyloxy)methyl)ethyl)amine; N-(3-(1H-imidazol-1-yl)propyl)-N-(2((9Z,12Z)-octadeca-9,12-dienyloxy)-1-(((9Z,12Z)-octadeca-9,12-dienyloxy)methyl)ethyl)amine; 1-methyl-4-(2((9Z,12Z)-octadeca-9,12-dienyloxy)-1-(((9Z,12Z)-octadeca-9,12-dienyloxy)methyl)ethyl)piperazine; 4-(2-((9Z,12Z)-octadeca-9,12-dienyloxy)-1-(((9Z,12Z)-octadeca-9,12-dienyloxy)methyl)ethyl)morpholine; N-(2-((9Z,12Z)-octadeca-9,12-dienyloxy)-1-(((9Z,12Z)-octadeca-9,12-dienyloxy)methyl)ethyl)-N-(3-pyrrolidin-1-ylpropyl)amine; N,N-dimethyl-N′-(2-((9Z,12Z)-octadeca-9,12-dienyloxy)-1-(((9Z,12Z)-octadeca-9,12-dienyloxy)methyl)ethyl)ethane-1,2-diamine; N-(2-(4-methylpiperazin-1-yl)ethyl)-N-(2-((9Z,12Z)-octadeca-9,12-dienyloxy)-1-(((9Z,12Z)-octadeca-9,12-dienyloxy)methyl)ethyl)amine; N-(2-(1H-imidazol-4-yl)ethyl)-N-(2-((9Z,12Z)-octadeca-9,12-dienyloxy)-1-(((9Z,12Z)-octadeca-9,12-dienyloxy)methyl)ethyl)amine; N,N-dimethyl-N-(3-(4-(2-((9Z,12Z)-octadeca-9,12-dienyloxy)-1-(((9Z,12Z)-octadeca-9,12-dienyloxy)methyl)ethyl)piperazin-1-yl)propyl)amine; 1,3-bis((9Z,12Z)-octadeca-9,12-dienyloxy)propan-2-amine; N-((1-methylpiperidin-4-yl)methyl)-N-(2-((9Z,12Z)-octadeca-9,12-dienyloxy)-1-(((9Z,12Z)-octadeca-9,12-dienyloxy)methyl)ethyl)amine; N-(2-((9Z,12Z)-octadeca-9,12-dienyloxy)-1-(((9Z,12Z)-octadeca-9,12-dienyloxy)methyl)ethyl)-N-(3-(pyrrolidin-1-ylmethyl)benzyl)amine; N-methyl-N-(2-((9Z,12Z)-octadeca-9,12-dienyloxy)-1-(((9Z,12Z)-octadeca-9,12-dienyloxy)methyl)ethyl)-N-(3-pyrrolidin-1-ylpropyl)amine; N-(3-((4-methylpiperazin-1-yl)methyl)benzyl)-N-(2((9Z,12Z)-octadeca-9,12-dienyloxy)-1-(((9Z,12Z)-octadeca-9,12-dienyloxy)methyl)ethyl)amine; N-methyl-N-((1-methylpiperidin-4-yl)methyl)-N-(2-((9Z,12Z)-octadeca-9,12-dienyloxy)-1-(((9Z,12Z)-octadeca-9,12-dienyloxy)methyl)ethyl)amine; N,N,N′-trimethyl-N′-(2-((9Z,12Z)-octadeca-9,12-dienyloxy)-1-(((9Z,12Z)-octadeca-9,12-dienyloxy)methyl)ethyl)propane-1,3-diamine; N-methyl-N-(2-((9Z,12Z)-octadeca-9,12-dienyloxy)-1-(((9Z,12Z)-octadeca-9,12-dienyloxy)methyl)ethyl)-N-(3-(pyrrolidin-1-ylmethyl)benzyl)amine; 1-(2-(1H-imidazol-1-yl)ethyl)-4-(2-((9Z,12Z)-octadeca-9,12-dienyloxy)-1-(((9Z,12Z)-octadeca-9,12-dienyloxy)methyl)ethyl)piperazine; N-(2-((9Z,12Z)-octadeca-9,12-dienyloxy)-1-(((9Z,12Z)-octadeca-9,12-dienyloxy)methyl)ethyl)-N-((2-pyrrolidin-1-ylpyridin-3-yl)methyl)amine; (9Z,9′Z,12Z,12′Z)-2-(4-methylpiperazin-1-yl)propane-1,3-diyl dioctadeca-9,12-dienoate; (9Z,9′Z,12Z,12′Z)-2-(3-(pyrrolidin-1-yl)propylamino)propane-1,3-diyl dioctadeca-9,12-dienoate; 1-methyl-4-(3((9Z,12Z)-octadeca-9,12-dienyloxy)-2-(((9Z,12Z)-octadeca-9,12-dienyloxy)methyl)propyl)piperazine; 1-(3-((9Z,12Z)-octadeca-9,12-dienyloxy)-2-(((9Z,12Z)-octadeca-9,12-dienyloxy)methyl)propyl)pyrrolidine; N-(3-aminopropyl)-N′-{3-[(2-[(9Z,12Z)-octadeca-9,12-dienyloxy]-1-{[(9Z,12Z)-octadeca-9,12-dienyloxy]methyl}ethyl)amino]propyl}butane-1,4-diamine; N-(3-[(9Z,12Z)-octadeca-9,12-dienyloxy]-2-{[(9Z,12Z)-octadeca-9,12-dienyloxy]methyl}propyl)-N-(3-pyrrolidin-1-ylpropyl)amine; N,N-dimethyl-N-(3-[(9Z,12Z)-octadeca-9,12-dienyloxy]-2-{[(9Z,12Z)-octadeca-9,12-dienyloxy]methyl}propyl)amine; 3-[(9Z,12Z)-octadeca-9,12-dienyloxy]-2-{[(9Z,12Z)-octadeca-9,12-dienyloxy]methyl}propyl 2-(diethylamino)ethylcarbamate; 3-[(9Z,12Z)-octadeca-9,12-dienyloxy]-2-{[(9Z,12Z)-octadeca-9,12-dienyloxy]methyl}propyl 2-pyrrolidin-1-ylethylcarbamate; 3-[(9Z,12Z)-octadeca-9,12-dienyloxy]-2-{[(9Z,12Z)-octadeca-9,12-dienyloxy]methyl}propyl 2-(dimethylamino)ethylcarbamate; 1-(2-[(9Z,12Z)-octadeca-9,12-dienyloxy]-1-{[(9Z,12Z)-octadeca-9,12-dienyloxy]methyl}ethyl)-4-(2-pyrrolidin-1-ylethyl)piperazine; N-(2-[(9Z)-octadec-9-enyloxy]-1-{[(9Z)-octadec-9-enyloxy]methyl}ethyl)-N-(3-pyrrolidin-1-ylpropyl)amine, 1-(2-[(9Z,12Z)-octadeca-9,12-dienyloxy]-1-{[(9Z,12Z)-octadeca-9,12-dienyloxy]methyl}ethyl)azetidine, 2-methyl-1-(2-[(9Z,12Z)-octadeca-9,12-dienyloxy]-1-{[(9Z,12Z)-octadeca-9,12-dienyloxy]methyl}ethyl)aziridine, 1-{3,4-bis[(9Z,12Z)-octadeca-9,12-dienyloxy]butyl}piperidine, 4-{3,4-bis[(9Z,12Z)-octadeca-9,12-dienyloxy]butyl}morpholine, N,N-diethyl-3,4-bis[(9Z,12Z)-octadeca-9,12-dienyloxy]butan-1-amine, N,N-dimethyl-3,4-bis[(9Z,12Z)-octadeca-9,12-dienyloxy]butan-1-amine, 1-{3,4-bis[(9Z,12Z)-octadeca-9,12-dienyloxy]butyl}-4-phenylpiperazine, 1-{3,4-bis[(9Z,12Z)-octadeca-9,12-dienyloxy]butyl}-4-methylpiperazine, N-(2-methoxyethyl)-N-methyl-3,4-bis[(9Z,12Z)-octadeca-9,12-dienyloxy]butan-1-amine, 1-{3,4-bis[(9Z,12Z)-octadeca-9,12-dienyloxy]butyl}-4-(2-methoxyphenyl)piperazine, N-{3,4-bis[(9Z,12Z)-octadeca-9,12-dienyloxy]butyl}-N,N′,N′-trimethylethane-1,2-diamine, N-{3,4-bis[(9Z,12Z)-octadeca-9,12-dienyloxy]butyl}-N-methyl-N-(2-pyridin-2-ylethyl)amine, N-benzyl-N-{3,4-bis[(9Z,12Z)-octadeca-9,12-dienyloxy]butyl}-N-methylamine, N-{3,4-bis[(9Z,12Z)-octadeca-9,12-dienyloxy]butyl}-N-(4-fluorobenzyl)-N-methylamine, 1-{3,4-bis[(9Z,12Z)-octadeca-9,12-dienyloxy]butyl}-4-(2-fluorophenyl)piperazine, N-benzyl-N-{3,4-bis[(9Z,12Z)-octadeca-9,12-dienyloxy]butyl}-N-ethylamine, N-{3,4-bis[(9Z,12Z)-octadeca-9,12-dienyloxy]butyl}-N-ethyl-N′,N′-dimethylethane-1,2-diamine, 1-{3,4-bis[(9Z,12Z)-octadeca-9,12-dienyloxy]butyl}-N,N-dimethylpiperidin-4-amine, 1-{3,4-bis[(9Z,12Z)-octadeca-9,12-dienyloxy]butyl}-N,N-dimethylpyrrolidin-3-amine, N,N-bis(2-methoxyethyl)-3,4-bis[(9Z,12Z)-octadeca-9,12-dienyloxy]butan-1-amine, 1-{3,4-bis[(9Z,12Z)-octadeca-9,12-dienyloxy]butyl}-4-methoxypiperidine, 1-{(3R)-3,4-bis[(9Z,12Z)-octadeca-9,12-dienyloxy]butyl}pyrrolidine, 1-{(3S)-3,4-bis[(9Z,12Z)-octadeca-9,12-dienyloxy]butyl}pyrrolidine, N-{(3R)-3,4-bis[(9Z,12Z)-octadeca-9,12-dienyloxy]butyl}-N,N-diethylamine, N-{(3S)-3,4-bis[(9Z,12Z)-octadeca-9,12-dienyloxy]butyl}-N,N-diethylamine, 1-{3,4-bis[(9Z,12Z)-octadeca-9,12-dienyloxy]butyl}pyrrolidine, N-(2-{3,4-bis[(9Z,12Z)-octadeca-9,12-dienyloxy]butoxy}ethyl)-N,N-diethylamine, 2-(2-{3,4-bis[(9Z,12Z)-octadeca-9,12-dienyloxy]butoxy}ethyl)-1-methylpyrrolidine, 1-(2-{3,4-bis[(9Z,12Z)-octadeca-9,12-dienyloxy]butoxy}ethyl)aziridine, 1-(2-{3,4-bis[(9Z,12Z)-octadeca-9,12-dienyloxy]butoxy}ethyl)-4-methylpiperazine, N-(2-{3,4-bis[(9Z,12Z)-octadeca-9,12-dienyloxy]butoxy}ethyl)-N,N-dimethylamine, 4-(diethylamino)-2-[(9Z,12Z)-octadeca-9,12-dienoyloxy]butyl (9Z,12Z)-octadeca-9,12-dienoate, 1-(2-{3,4-bis[(9Z,12Z)-octadeca-9,12-dienyloxy]butoxy}ethyl)pyrrolidine, N,N-diethyl-N-(2-{2-[(8Z,11Z)-heptadeca-8,11-dienyl]-2-[(9Z,12Z)-octadeca-9,12-dienyl]-1,3-dioxolan-4-yl}ethyl)amine, 1-{[(9Z)-octadec-9-enoyloxy]methyl}-3-pyrrolidin-1-ylpropyl (9Z)-octadec-9-enoate, 1-{3,4-bis[(9Z)-octadec-9-enyloxy]butyl}pyrrolidine, 1-{[(5Z,8Z,11Z,14Z)-icosa-5,8,11,14-tetraenoyloxy]methyl}-3-pyrrolidin-1-ylpropyl (5Z,8Z,11Z,14Z)-icosa-5,8,11,14-tetraenoate, (3S)-3,4-bis[(9Z,12Z)-octadeca-9,12-dienyloxy]butyl 3-pyrrolidin-1-ylpropylcarbamate, 1-[3,4-bis(octadecyloxy)butyl]pyrrolidine, 1-[3,4-bis(hexadecyloxy)butyl]pyrrolidine, 1-{3,4-bis[(9E)-hexadec-9-enyloxy]butyl}pyrrolidine, 1-{3,4-bis[(9E)-octadec-9-enyloxy]butyl}pyrrolidine, 1-{3,4-bis[(9E,12E)-octadeca-9,12-dienyloxy]butyl}pyrrolidine, 1-{3,4-bis[(9Z,12Z,15Z)-octadeca-9,12,15-trienyloxy]butyl}pyrrolidine, N¹-{(3S)-3,4-bis[(9Z,12Z)-octadeca-9,12-dienyloxy]butyl}-N³,N³-diethyl-beta-alaninamide, N-{3,4-bis[(9Z,12Z)-octadeca-9,12-dienyloxy]butyl}-N-[3-(1H-imidazol-1-yl)propyl]amine, N-{3,4-bis[(9Z,12Z)-octadeca-9,12-dienyloxy]butyl}-N,N′,N′-trimethylpropane-1,3-diamine, 1-(1-{3,4-bis[(9Z,12Z)-octadeca-9,12-dienyloxy]butyl}pyrrolidin-3-yl)-1H-imidazole, N-{3,4-bis[(9Z,12Z)-octadeca-9,12-dienyloxy]butyl}-N-(3-pyrrolidin-1-ylpropyl)amine, N-{3,4-bis[(9Z,12Z)-octadeca-9,12-dienyloxy]butyl}-N′,N′-dimethylpropane-1,3-diamine, 1-{3,4-bis[(9Z,12Z)-octadeca-9,12-dienyloxy]butyl}azetidine, 1-{3,4-bis[(9Z,12Z)-octadeca-9,12-dienyloxy]butyl}-2-methylpyrrolidine, 1-{3,4-bis[(9Z,12Z)-octadeca-9,12-dienyloxy]butyl}-2,5-dimethylpyrrolidine, are 1-(2,3-bis((9Z,12Z)-octadeca-9,12-dienyloxy)propyl)pyrrolidine, 1-(2,3-bis((9Z,12Z)-octadeca-9,12-dienyloxy)propyl)-1H-imidazole, 1-(2,3-bis((9Z,12Z)-octadeca-9,12-dienyloxy)propyl)-4-methylpiperazine, 1-(2,3-bis((9Z,12Z)-octadeca-9,12-dienyloxy)propyl)-4-methyl-1,4-diazepane, 1-(2,3-bis((9Z,12Z)-octadeca-9,12-dienyloxy)propyl)-4-phenylpiperazine, 1-(2,3-bis((9Z,12Z)-octadeca-9,12-dienyloxy)propyl)-4-pyridin-2-ylpiperazine, 1-(2,3-bis((9Z,12Z)-octadeca-9,12-dienyloxy)propyl)piperidine, 4-(2,3-bis((9Z,12Z)-octadeca-9,12-dienyloxy)propyl)morpholine, 1-((2R)-2,3-bis((9Z,12Z)-octadeca-9,12-dienyloxy)propyl)pyrrolidine, 1-((2S)-2,3-bis((9Z,12Z)-octadeca-9,12-dienyloxy)propyl)pyrrolidine, 1-(2,3-bis((9Z,12Z)-octadeca-9,12-dienyloxy)propyl)-4-ethylpiperazine, N-(2,3-bis((9Z,12Z)-octadeca-9,12-dienyloxy)propyl)-N-methyl-N-(3-(pyrrolidin-1-ylmethyl)benzyl)amine, N-(2-(4-(2,3-bis((9Z,12Z)-octadeca-9,12-dienyloxy)propyl)piperazin-1-yl)ethyl)-N,N-dimethylamine, 1-((2S)-2,3-bis((9Z,12Z)-octadeca-9,12-dienyloxy)propyl)-4-methylpiperazine, 1-((2R)-2,3-bis((9Z,12Z)-octadeca-9,12-dienyloxy)propyl)-4-methylpiperazine, 1-(2,3-bis((9Z,12Z)-octadeca-9,12-dienyloxy)propyl)-4-(2-pyrrolidin-1-ylethyl)piperazine, 2-(4-(2,3-bis((9Z,12Z)-octadeca-9,12-dienyloxy)propyl)piperazin-1-yl)pyrimidine, 1-(2,3-bis((9Z,12Z)-octadeca-9,12-dienyloxy)propyl)-N,N-diethylpyrrolidin-3-amine, 1-((9Z,12Z)-octadeca-9,12-dienyloxy)-3-pyrrolidin-1-ylpropan-2-ol, 2-[(9Z,12Z)-octadeca-9,12-dienoyloxy]-1-(pyrrolidin-1-ylmethyl)ethyl (9Z,12Z)-octadeca-9,12-dienoate, 2-[(9Z,12Z)-octadeca-9,12-dienyloxy]-1-(pyrrolidin-1-ylmethyl)ethyl (9Z,12Z)-octadeca-9,12-dienoate, 1-({2-[(8Z,11Z)-heptadeca-8,11-dienyl]-2-[(9Z,12Z)-octadeca-9,12-dienyl]-1,3-dioxolan-4-yl}methyl)pyrrolidine, 1-{2,3-bis[(5Z,8Z,11Z,14Z)-icosa-5,8,11,14-tetraenyloxy]propyl}pyrrolidine, 1-{3-[(5Z,8Z,11Z,14Z)-icosa-5,8,11,14-tetraenyloxy]-2-[(9Z,12Z)-octadeca-9,12-dienyloxy]propyl}pyrrolidine, 1-{2,3-bis[(9E,12E)-octadeca-9,12-dienyloxy]propyl}pyrrolidine, 1-{2-[(9E,12E)-octadeca-9,12-dienyloxy]-3-[(9Z,12Z)-octadeca-9,12-dienyloxy]propyl}pyrrolidine, 1-[2,3-bis(tetradecyloxy)propyl]pyrrolidine, 1-[2,3-bis(octadecyloxy)propyl]pyrrolidine, 1-{2,3-bis[(9Z)-octadec-9-enyloxy]propyl}pyrrolidine, 1-[2,3-bis(dodecyloxy)propyl]pyrrolidine, 1-{2,3-bis[(9Z,12Z)-octadeca-9,12-dienyloxy]propyl}pyrrolidin-3-ol, 1-{3-[(9Z,12Z)-hexadeca-9,12-dienyloxy]-2-[(9Z)-octadec-9-enyloxy]propyl}pyrrolidine, 1-{2,3-bis[(9Z,12Z)-octadeca-9,12-dienyloxy]propyl}-N,N-dimethylpyrrolidin-3-amine and 1-[3-[(9Z,12Z)-hexadeca-9,12-dienyloxy]-2-(tetradecyloxy)propyl]pyrrolidine, and mixtures thereof.

Lipid-Based Particles are a mixture of one or more PEG-lipid conjugates of Formula (I), one or more non-cationic lipids, one or more cationic lipids, and one or more therapeutic agents. Specific Lipid-Based Particles comprise the following lipid mixtures: cationic lipid(s) (about 2-60% by weight), non-cationic lipid(s) (about 5-90% by weight), and PEG-lipid conjugate(s) (about 0.1-20%).

### Data

### Tables 1 and 2

### Representative In-Vitro Lipid-Based Particles

TABLE 1

| Therapeutic | | |
|---|---|---|
| Agent | Mass (mg) | Vol (mL in water 10 mg/mL) |
| TetR-siRNA | 3.0 | 0.3 |
| Total Lipids | 75 | 7.5 |

TABLE 2

| | Wt % | Mass (mg) | Vol (μL in ethanol, 10 mg/mL) |
|---|---|---|---|
| PEG-lipid | 9 | 6.75 | |
| conjugate | | | |
| DSPC | 14 | 10.5 | 1050 |
| Cholesterol | 33 | 24.75 | 2475 |
| Cationic lipid | 44 | 33 | 3300 |

TABLE 3
Representative In vivo Lipid-Based Particles

| Therapeutic | | |
|---|---|---|
| Agent | Mass (mg) | Vol (mL in water 10 mg/mL) |
| TetR-siRNA | 5.0 | 0.5 |
| Total Lipids | 125 | 12.5 |

| | Cationic | | | | |
|---|---|---|---|---|---|
| | lipid | PEG-lipid | Non-cationic lipid | Cholesterol | Total |
| % (w/w) | 44 | 9 | 14 | 33 | 100 |
| Weight | 54.9 | 11.2 | 17.6 | 41.4 | |
| (mg) | | | | | |
| Volume | 5.49 | 1.12 | 1.76 | 4.14 | |
| (ml) | | | | | |

### Preparation of Lipid-Based Particles

The mixing solution of cationic lipids, cholesterol, non-cationic lipids and PEG-lipids was prepared in ethanol (total concentration at 10 mg/mL). siSTABLE (purchased from ThermoFisher) (sense-5′ GGG GAA AGC UGG CAA GAU UUU-3′ SEQ ID NO. 1, antisense-5′-AAU CUU GCC AGC UUU CCC CUU-3′ SEQ ID NO: 2) % stock solution was prepared in 10 mg/mL of solution by dissolving 10 mg siRNA in 1 mL of RNAse-free UltraPure Water. The calculated amount of siRNA solution was added to 1 mL of citrate buffer (pH 4.0, 20 mM), to provide an siRNA concentration of 0.2 mg/mL, and warmed to 60° C. The calculated amount of lipid solution was warmed to 60° C., transferred to a 0.5 mL syringe with 28½ gauge needle, and injected into the citrate buffer with stirring at 60° C. After 3 minutes, 3 mL of PBS solution at room temperature (pH 7.4) was added into the lipid mixture with stirring. The Lipid-Based Particle solution was cooled to room temperature.

### Analysis of Lipid-Based Particles

The siRNA concentrations were measured using Quanti-iT RiboGreen RNA reagent (Molecular Probes, (R11490)). Vesicle sizes were characterized by dynamic light scattering with a DynaPro™ Plate Reader (Wyatt Technology) in 96-well half-area UV plate (Coring) after diluting the formulation sample (20 μL) in phosphate buffered saline (80 μL) at a pH of about 7-8. A 1% agarose gel-based assay was used for analyzing nuclease degradation and protection. Encapsulation efficiency (EE) was calculated using data obtained from a RiboGreen assay.

### Ribogreen Assay for Measuring siRNA Concentration and Encapsulation Efficiency of Lipid-Based Particles

RNA concentration and encapsulation efficiency were determined using a Quant-iT® Ribogreen RNA reagent and kit available from Invitrogen. The siRNA was released from the Lipid-Based Particle using one of the following reagents: ethanol, Triton X-100, or phenol/chloroform. The siRNA concentration is quantified using fluorescent reading at 480 nm/520 nm.

### Particle Sizing Assay

Particle sizes and size distributions (PDI) were characterized by using dynamic light scattering (DLS). A DLS plate reader (Dynapro™, Wyatt Technology) was used for the DLS measurement. This DLS plate reader uses an 830 nm laser and the scattering angle is 158°. It also can control temperature from 4° C. to 70° C. A 96-well format was employed for the samples.

Samples for DLS analysis were prepared by mixing 20 μL of each sample stock solution with 80 μL PBS directly in the 96-well plate (#3697, Corning). Sample mixing was accomplished using a microplate shaker (Orbis, Mikura Ltd.). Plates were read at 20° C. with an acquisition time of 50 seconds for each sample, and data was analyzed with Wyatt Technology's Dynamics V6 software. To rule out potential multiple scattering artifacts, a second plate at 4-fold reduced sample concentrations was independently prepared by mixing 5 μL stock solutions with 95 μL PBS. Under our experimental conditions the results at the two concentrations were very similar, and the final reported result for each sample represents the average of values obtained from the two plates.

TABLE 4
Data Table Of Particle Size And Encapsulation Efficiency

| Lipid- | | | | | |
|---|---|---|---|---|---|
| Based | | | | Particle | |
| Particle | | Lipid ratio (wt | Lipid:siRNA | Size | Encapsulation |
| No. | Formulation | %) | ratio | (d/nm) | Efficiency (%) |
| 1 | Example 14/Example | 45/10/15/30 | 25:1 | 152 | 96 |
| | 3/DSPC/chol | | | | |
| 2 | Example 14/Example | 45/10/15/30 | 25:1 | 164 | 97 |
| | 4/DSPC/chol | | | | |
| 3 | Example 13/Example | 45/10/15/30 | 25:1 | 116 | 92 |
| | 2/DSPC/chol | | | | |
| 4 | Example 13/Example | 44/9/14/33 | 25:1 | 110 | 91 |
| | 3/DSPC/chol | | | | |
| 5 | Example 13/Example | 44/9/14/33 | 25:1 | 100 | 84 |
| | 12/DSPC/chol | | | | |
| 6 | Example 13/Example | 44/9/14/33 | 25:1 | 118 | 94 |
| | 8/DSPC/chol | | | | |
| 7 | Example 13/Example | 44/9/14/33 | 25:1 | 99 | 94 |
| | 11/DSPC/chol | | | | |
| 8 | Example 13/Example | 44/9/14/33 | 25:1 | 99 | 95 |
| | 15/DSPC/chol | | | | |
| 9 | Example 13/Example | 44/9/14/33 | 25:1 | 99 | 95 |
| | 17/DSPC/chol | | | | |
| 10 | Example 13/Example | 44/9/14/33 | 25:1 | 170 | 91 |
| | 19/DSPC/chol | | | | |
| 11 | Example 13/Example | 44/9/14/33 | 25:1 | 85 | 94 |
| | 20/DSPC/chol | | | | |
| 12 | Example 13/Example | 44/9/14/33 | 25:1 | 108 | 93 |
| | 21/DSPC/chol | | | | |
| 13 | Example 13/Example | 44/4.5/4.5/14/33 | 25:1 | 119 | 97 |
| | 3/PEG-DSG/DSPC/chol | | | | |
| 14 | Example 13/Example | 44/4.5/4.5/14/33 | 25:1 | 120 | 94 |
| | 3/PEG-DSPE/DSPC/chol | | | | |
| 15 | Example 13/Example | 46/5/15/32 | 25:1 | 173 | 89 |
| | 3/DSPC/chol | | | | |
| 16 | Example 13/Example | 44/13/13/30 | 25:1 | 86 | 99 |
| | 3/DSPC/chol | | | | |
| 17 | Example 13/Example | 44/9/14/33 | 15:1 | 88 | 99 |
| | 3/DSPC/chol | | | | |
| 18 | Example 13/Example | 44/9/14/33 | 10:1 | 84 | 97 |
| | 3/DSPC/chol | | | | |

### Tumor Models

The animal studies were carried out in accordance with internal Institutional Animal Care and Use Committee (IACUC) guidelines at Abbott Laboratories. Scid female mice at 6 to 8 weeks of age were obtained from Charles River Laboratory and used for intraliver tumor models. Mouse livers were exposed by vertical incision on mouse abdomens and the tumor cells were directly injected into the livers. The incision was closed by suture and wound clips. All cell lines used for creating xenograft tumors were subjected to the IMPACT profile I test (18 agents) at the University of Missouri Research Animal Diagnostic and Investigative Laboratory, and all cell lines were found negative for the 18 infectious agents tested. Tumor cells were suspended in a 1:1 mixture of S-MEM (Invitrogen, Carlsbad, Calif.) and matrigel (BD Bioscience, San Jose, Calif.) and inoculated at 1×10E6 cells per animal.

### Animal Dosing and Sample Harvesting

Treatments were started 3˜4 weeks after tumor inoculation. Formulated or unformulated siRNAs were administrated via tail vein (i.v) injection.

### In Vivo Procedure to Determine Efficacy of Lipid-Based Particles

The in vivo knockdown activities of formulations were tested using Abbott's positive readout system (MDA-TetR-Luc cells). Liver tumors were established by direct inoculation of tumor cells into the liver of SCID female mice (Charles River). 14 to 20 days later, the background bioluminescence of the tumors were measured by IVIS Imaging System (Caliper Life Science) and the mice were signal-matched.

Formulated siRNAs were delivered through tail vein at 0.2 mL per mouse, equivalent to 2.5 mg/kg of siRNA. As a positive and negative control, TetR and non-target-composition (NTC) siRNAs were formulated in a benchmark formulations and included in the studies.

Mice were dosed at day 1 and 2, the bioluminescence were recorded on day 1 (before dosing) and day 4. The ratio of bioluminescence of day 4 vs day 1 was calculated for each animal and an increase indicates target knockdown.

### Bioluminescence Imaging and Analysis

In vivo bioluminescence imaging and analysis were conducted on the IVIS 200 system using the Living Image acquisition and analysis software (Caliper Life Science, Hopkinton, Mass.). After intra-peritoneal injection of luciferin (Promege, Madison, Wis.) at 150 mg/kg, mice were anesthetized with isofluorane. Four minutes after the injection of luciferin, a series of time-lapse images were acquired at 2 minutes intervals in a total of 10 minutes. Regions of interest (ROI) were drawn around the tumors and signal intensity was quantified as the sum of photon counts per second within the ROI after the subtraction of background luminescence. The peak reading during the 10-minute imaging period was used for calculating the signal ratio before and after siRNA delivery.

### Procedures to Examine Liver Function

To exam liver function, the activity of liver enzymes were measured, which included AST (serum aspartate aminotransferase), ALT (serum alanine aminotransferase) and ALP (alkaline phosphatase). The increase in the activity of all three enzymes suggests liver damage and the degree of increase positively correlates with the grade of liver toxicity. Naïve mice (SCID female, age 13-15 weeks, Charles River Labs) were i.v. dosed with siRNA formulations through the tail vein at the indicated dose, volume and frequency of Table 5 On the second day after the last dose, mouse serum was harvested to exam liver function by testing liver enzyme activities. The enzymes tested include AST (serum aspartate aminotransferase), ALT (serum alanine aminotransferase) and ALP (alkaline phosphatase). All assays were done on Abbott Aeroset Automated Chemistry Analyzer (Abbott Diagnostic) with corresponding kits (AST, cat #7D81-20; ALT, cat #7D56-20 and ALP, cat #7D55-21, all are products of Abbott Diagnostic) following the manufacturer's protocol. Results are shown in Table 6. Elevation of all three enzymes correlates to liver damage and the degree of elevation positively correlates with the grade of liver toxicity. Necropsy analysis was done on animals and the results are shown in Table 7.

TABLE 5
Lipid Based Particle Formulations

| | | | | | Dosing | | |
|---|---|---|---|---|---|---|---|
| | | Lipid Ratio | Lipid:siRNA | Dosing | vol | | |
| # | Formulation | (wt %) | ratio | Schedule | (ml) | mg/kg | n |
| 1 | Example | 44/9/14/33 | 25:01 | QDx2 | 0.2 | 2.5 | 2 |
| | 13/Example | | | | | | |
| | 3/DSPC/Chol | | | | | | |
| 2 | Example | 44/9/14/33 | 25:01 | QDx2 | 0.4 | 5 | 3 |
| | 13/Example | | | | | | |
| | 3/DSPC/Chol | | | | | | |
| 3 | Example | 44/9/14/33 | 25:01 | QDx2 | 0.8 | 10 | 3 |
| | 13/Example | | | | | | |
| | 3/DSPC/Chol | | | | | | |
| 4 | Example | 44/9/14/33 | 25:01 | QDx2 | 0.2 | 2.5 | 2 |
| | 23/Example | | | | | | |
| | 22/DSPC/Chol | | | | | | |
| 5 | Example | 44/9/14/33 | 25:01 | QDx2 | 0.4 | 5 | 3 |
| | 23/Example | | | | | | |
| | 22/DSPC/Chol | | | | | | |
| 6 | Example | 44/9/14/33 | 25:01 | QDx2 | 0.8 | 10 | 3 |
| | 23/Example | | | | | | |
| | 22/DSPC/Chol | | | | | | |
| 7 | Example | 44/9/14/33 | 25:01 | QDx2 | 0.2 | 2.5 | 2 |
| | 13/Example | | | | | | |
| | 16/DSPC/Chol | | | | | | |
| 8 | Example | 44/9/14/33 | 25:01 | QDx2 | 0.4 | 5 | 3 |
| | 13/Example | | | | | | |
| | 16/DSPC/Chol | | | | | | |
| 9 | Example | 44/9/14/33 | 25:01 | QDx2 | 0.8 | 10 | 3 |
| | 13/Example | | | | | | |
| | 16/DSPC/Chol | | | | | | |
| 10 | Example | 44/9/14/33 | 25:01 | QDx2 | 0.2 | 2.5 | 2 |
| | 13/Example | | | | | | |
| | 2/DSPC/Chol | | | | | | |
| 11 | Example | 44/9/14/33 | 25:01 | QDx2 | 0.4 | 5 | 3 |
| | 13/Example | | | | | | |
| | 2/DSPC/Chol | | | | | | |
| 12 | Example | 44/9/14/33 | 25:01 | QDx2 | 0.8 | 10 | 3 |
| | 13/Example | | | | | | |
| | 2/DSPC/Chol | | | | | | |
| 13 | Example 13/Example | 44/9/14/33 | 25:01 | QDx2 | 0.2 | 2.5 | 2 |
| | 4/DSPC/Chol | | | | | | |
| 14 | Example 13/Example | 44/9/14/33 | 25:01 | QDx2 | 0.4 | 5 | 3 |
| | 4/DSPC/Chol | | | | | | |
| 15 | Example 13/Example | 44/9/14/33 | 25:01 | QDx2 | 0.8 | 10 | 3 |
| | 4/DSPC/Chol | | | | | | |
| 16 | Example | 44/9/14/33 | 25:1 | QDx2 | 0.2 | 2.5 | 3 |
| | 23/Example | | | | | | |
| | 22/DSPC/Chol | | | | | | |
| 17 | Example | 44/9/14/33 | 25:1 | QDx2 | 0.4 | 5 | 3 |
| | 23/Example | | | | | | |
| | 22/DSPC/Chol | | | | | | |
| 18 | Example | 44/9/14/33 | 25:1 | QDx2 | 0.8 | 10 | 3 |
| | 23/Example | | | | | | |
| | 22/DSPC/Chol | | | | | | |
| 19 | Example | 44/9/14/33 | 25:1 | QDx2 | 0.2 | 2.5 | 3 |
| | 13/Example | | | | | | |
| | 3/DSPC/Chol | | | | | | |
| 20 | Example | 44/9/14/33 | 25:1 | QDx2 | 0.4 | 5 | 3 |
| | 13/Example | | | | | | |
| | 3/DSPC/Chol | | | | | | |
| 21 | Example | 44/9/14/33 | 25:1 | QDx2 | 0.8 | 10 | 3 |
| | 13/Example | | | | | | |
| | 3/DSPC/Chol | | | | | | |
| 22 | Example | 44/9/14/33 | 25:1 | QDx2 | 0.2 | 2.5 | 3 |
| | 13/Example | | | | | | |
| | 22/DSPC/Chol | | | | | | |
| 23 | Example | 44/9/14/33 | 25:1 | QDx2 | 0.4 | 5 | 3 |
| | 13/Example | | | | | | |
| | 22/DSPC/Chol | | | | | | |
| 24 | Example | 44/9/14/33 | 25:1 | QDx2 | 0.8 | 10 | 3 |
| | 13/Example | | | | | | |
| | 22/DSPC/Chol | | | | | | |
| 25 | Example | 44/9/14/33 | 25:1 | QDx2 | 0.2 | 2.5 | 3 |
| | 23/Example | | | | | | |
| | 3/DSPC/Chol | | | | | | |
| 26 | Example | 44/9/14/33 | 25:1 | QDx2 | 0.4 | 5 | 3 |
| | 23/Example | | | | | | |
| | 3/DSPC/Chol | | | | | | |
| 27 | Example | 44/9/14/33 | 25:1 | QDx2 | 0.8 | 10 | 3 |
| | 23/Example | | | | | | |
| | 3/DSPC/Chol | | | | | | |

TABLE 6
Liver Function Analysis

| # | ALP (U/L) | ALT (U/L) | AST (U/L) |
|---|---|---|---|
| 1 | 48.0 | 30.0 | 118.5 |
| 2 | 55.67 | 38.0 | 203.67 |
| 3 | 66.67 | 118.0 | 482.67 |
| 4 | 58.80 | 45.50 | 121.0 |
| 5 | 96.67 | 4981.0 | 7239.5 |
| 6 | 587.0 | >4700 | 9253.0 |
| 7 | 69.0 | 47.0 | 199.0 |
| 8 | 457.0 | >4000 | 7846.0 |
| 9 | * | * | * |
| 10 | 46.5 | 34.0 | 91.5 |
| 11 | 46.0 | 480.67 | 509.0 |
| 12 | 498.67 | >4700 | 8754.5 |
| 13 | 40 | 42 | 143.5 |
| 14 | 54 | 45.33 | 227 |
| 15 | 90.33 | 47 | 383.67 |
| 16 | 75.0 | 38.7 | 126.0 |
| 17 | 115.0 | >4700 | 8025.0 |
| 18 | 414.5 | >4700 | 11582.5 |
| 19 | 55.7 | 36.0 | 147.0 |
| 20 | 58.3 | 39.3 | 198.3 |
| 21 | 100.5 | 348.5 | 702.0 |
| 22 | 70.7 | 31.0 | 104.0 |
| 23 | 214.7 | >4700 | 10795.0 |
| 24 | 682.0 | 3539.0 | 6770.0 |
| 25 | 53.3 | 28.3 | 86.7 |
| 26 | 32.3 | 37.0 | 145.0 |
| 27 | 66.0 | 374.3 | 779.7 |

Note:
For 6, 8, 12, 17, 18, and 23, the ALT level is over the detecting limit and the reading over 4700 (U/L).
* for # 9, all of subjects were found dead before the harvest date.

TABLE 7
Liver Morphology

| # | mg/kg | liver 1 | liver 2 |
|---|---|---|---|
| 5 | 5 | ++ | ++ |
| 8 | 5 | +++ | +++ |
| 11 | 5 | + | + |
| 2 | 5 | normal | normal |
| 14 | 5 | normal | normal |
| 17 | 5 | ++ | ++ |
| 20 | 5 | normal | normal |
| 23 | 5 | ++ | ++ |
| 26 | 5 | normal | normal |

+ slightly paled color
++ more discoloration, tan-yellow
+++ highest degree of discoloration, swollen and brittle

As shown in Table 6 and Table 7, the liver function analysis and liver morphology data indicate that formulations containing PEG-lipid conjugates of this invention show an improved liver toxicity profile compared to formulations known in the art.

### Synthesis

The following abbreviations have the meanings indicated: ADDP means 1,1′-(azodicarbonyl)dipiperidine; AD-mix-β means a mixture of (DHQD)₂PHAL, K₃Fe(CN)₆, K₂CO₃ and K₂SO₄); AIBN means 2,2′-azobis(2-methylpropionitrile); 9-BBN means 9-borabicyclo(3.3.1)nonane; Cp means cyclopentadiene; (DHQD)₂PHAL means hydroquinidine 1,4-phthalazinediyl diethyl ether; DBU means 1,8-diazabicyclo(5.4.0)undec-7-ene; DCC means dicyclohexylcarbodiimide; DIBAL means diisobutylaluminum hydride; DIEA means diisopropylethylamine; DMAP means N,N-dimethylaminopyridine; DME means 1,2-dimethoxyethane; DMF means N,N-dimethylformamide; dmpe means 1,2-bis(dimethylphosphino)ethane; DMSO means dimethylsulfoxide; dppa means diphenylphosphoryl azide; dppb means 1,4-bis(diphenylphosphino)butane; dppe means 1,2-bis(diphenylphosphino)ethane; dppf means 1,1′-bis(diphenylphosphino)ferrocene; dppm means 1,1-bis(diphenylphosphino)methane; EDAC means 1-(3-dimethylaminopropyl)-3-ethylcarbodiimide; Fmoc means fluorenylmethoxycarbonyl; HATU means O-(7-azabenzotriazol-1-yl)-N,N′N′N′-tetramethyluronium hexafluorophosphate; HMPA means hexamethylphosphoramide; IPA means isopropyl alcohol; LDA means lithium diisopropylamide; LHMDS means lithium bis(hexamethyldisilylamide); MP-BH₃ means macroporus triethylammonium methylpolystyrene cyanoborohydride; LAH means lithium aluminum hydride; NCS means N-chlorosuccinimide; PyBOP means benzotriazol-1-yloxytripyrrolidinophosphonium hexafluorophosphate; TDA-1 means tris(2-(2-methoxyethoxy)ethyl)amine; TEA means triethylamine; TFA means trifluoroacetic acid; THF means tetrahydrofuran; NCS means N-chlorosuccinimide; NMM means N-methylmorpholine; NMP means N-methylpyrrolidine; PPh₃ means triphenylphosphine.

The following schemes are presented to provide what is believed to be the most useful and readily understood description of procedures and conceptual aspects of this invention. Compounds of this invention may be made by synthetic chemical processes, examples of which are shown herein. It is meant to be understood that the order of the steps in the processes may be varied, that reagents, solvents and reaction conditions may be substituted for those specifically mentioned, and that vulnerable moieties may be protected and deprotected, as necessary.

### Schemes

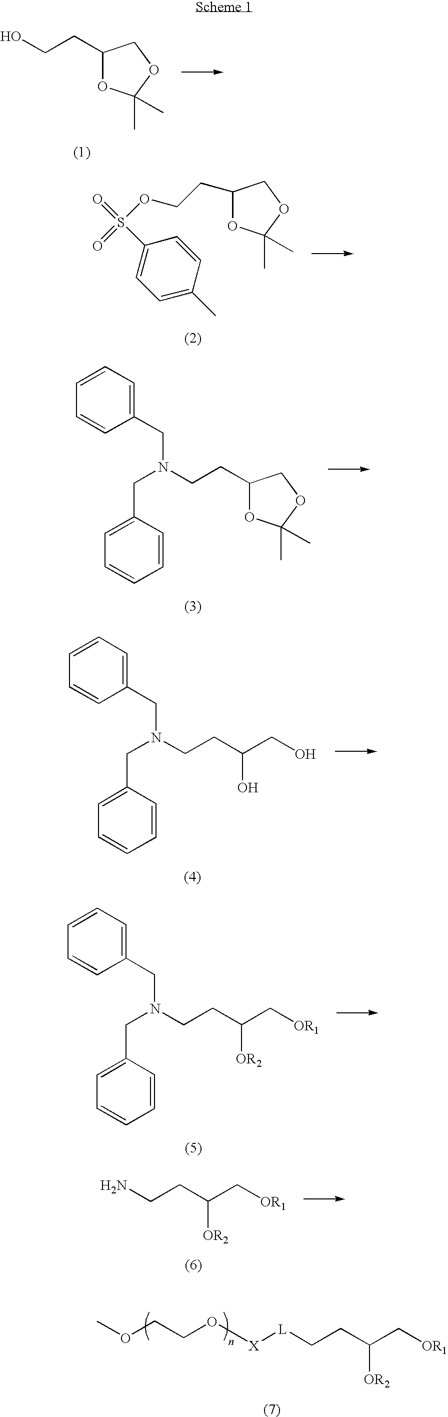

As shown in Scheme 1,2-(2,2-dimethyl-1,3-dioxolan-4-yl)ethanol (1) can be reacted with tosyl chloride in the presence of a base such as but not limited to triethylamine and a catalyst such as but not limited to 4-(dimethylamino)pyridine, to provide 2-(2,2-dimethyl-1,3-dioxolan-4-yl)ethyl 4-methylbenzenesulfonate (2). The reaction is typically performed in a solvent such as but not limited to dichloromethane at 0° C. before warming up to room temperature. N,N-Dibenzyl-2-(2,2-dimethyl-1,3-dioxolan-4-yl)ethanamine (3) can be prepared from 2-(2,2-dimethyl-1,3-dioxolan-4-yl)ethyl 4-methylbenzenesulfonate (2) by reacting the latter with dibenzylamine. The reaction is typically conducted at elevated temperatures and may be conducted in a single mode microwave instrument. N,N-Dibenzyl-2-(2,2-dimethyl-1,3-dioxolan-4-yl)ethanamine (3) can be reacted with an acid such as but not limited to aqueous hydrochloric acid to provide 4-(dibenzylamino)butane-1,2-diol (4). The reaction is typically conducted at ambient temperature in a solvent such as but not limited to tetrahydrofuran. A compound of Formula (5), wherein R²=R¹, can be prepared from 4-(dibenzylamino)butane-1,2-diol (4) by reacting 4-(dibenzylamino)butane-1,2-diol (4) with R¹OSO₂CH₃ in the presence of a strong base such as but not limited to sodium hydride. The reaction is typically performed at elevated temperatures in a solvent such as but not limited to tetrahydrofuran. A compound of Formula (5) can be reacted with hydrogen gas in the presence of a catalyst such as but not limited to palladium on carbon to provide a compound of Formula (6). The reaction is typically conducted at ambient temperature in a solvent such as but not limited to methanol, dichloromethane, ethyl acetate, or mixtures thereof. Compounds of Formula (7), which are representative of compounds of this invention wherein R²=R¹, can be prepared from compounds of Formula (6) using an appropriate PEGylation reagent in the presence of a base such as but not limited to triethylamine, or Hunig's base. The reaction is typically conducted at ambient temperature in a solvent such as but not limited to dichloromethane.

Alternatively, compounds of Formula (7), wherein R¹ and R² are C(O)R³, can be prepared as shown in Scheme 1 and described above, except a compound of Formula R³COOH can be reacted with 4-(dibenzylamino)butane-1,2-diol (4) to provide a compound of Formula (5) wherein R¹ and R² are C(O)R³. The reaction can be performed using coupling conditions known by those skilled in the art and readily available in the literature.

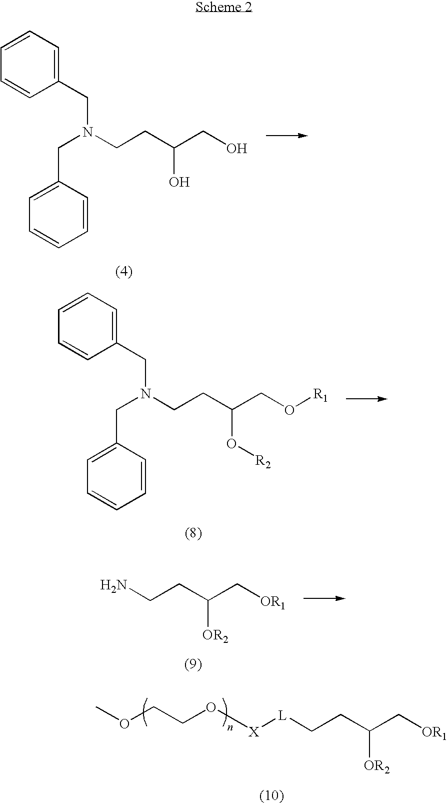

As shown in Scheme 2,4-(dibenzylamino)butane-1,2-diol (4) can be reacted first with R¹Br, in the presence of a strong base such as sodium hydride, followed by reaction with R²OSO₂CH₃ in the presence of a strong base such as sodium hydride, to provide a compound of Formula (8), wherein R¹≠R². Both reactions typically require an elevated temperature and a solvent such as but not limited to tetrahydrofuran, N,N-dimethylformamide, or mixtures thereof. A compound of Formula (8) can be reacted with hydrogen gas in the presence of a catalyst such as but not limited to palladium on carbon to provide a compound of Formula (9). The reaction is typically conducted at ambient temperature in a solvent such as but not limited to methanol, dichloromethane, ethyl acetate, or mixtures thereof. Compounds of Formula (10), which are representative of compounds of this invention, wherein R²≠R¹, can be prepared from compounds of Formula (9) using an appropriate PEGylation reagent in the presence of a base such as but not limited to triethylamine, or Hunig's base. The reaction is typically conducted at ambient temperature in a solvent such as but not limited to dichloromethane.

The following examples are presented to provide what is believed to be the most useful and readily understood description of procedures and conceptual aspects of this invention. The exemplified compounds were named using ACD/ChemSketch Version 5.06 (5 Jun. 2001, Advanced Chemistry Development Inc., Toronto, Ontario), or ChemDraw® Ver. 9.0.5 (CambridgeSoft, Cambridge, Mass.) except for Example 20, which was named using Marvin Version 5.1 (ChemAxon Kft., Budapest, Hungary). Intermediates were named using ChemDraw® Ver. 9.0.5 (CambridgeSoft, Cambridge, Mass.).

### EXAMPLES

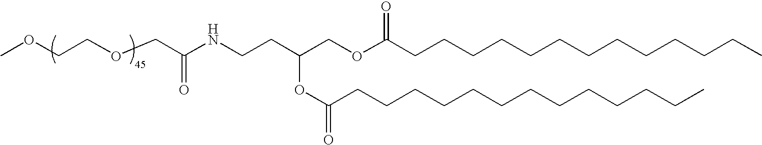

### Example 1

### 6-oxo-2-(tetradecanoyloxy)-8,11,14,17,20,23,26,29,32,35,38,41,44,47,50,53,56,59,62,65,68,71,74,77,80,83,86,89,92, 95,98,101,104,107,110,113,116,119,122,125,128,131,134,137,140,143-hexatetracontaoxa-5-a zatetratetracontahect-1-yl myristate

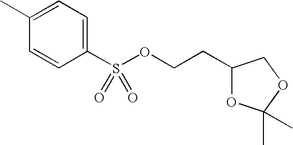

### Example 1A

### 2-(2,2-dimethyl-1,3-dioxolan-4-yl)ethyl 4-methylbenzenesulfonate

2-(2,2-Dimethyl-1,3-dioxolan-4-yl)ethanol (5 g) was added to dichloromethane (86 ml) and the mixture was cooled to 0° C. To this solution was added triethylamine (6.9 g, 9.6 ml), tosyl chloride (6.5 g) and 4-(dimethylamino)pyridine (0.42 g). The mixture stirred at room temperature overnight. The mixture was quenched with saturated NH₄Cl and diluted with ethyl acetate. The aqueous layer was extracted twice with ethyl acetate and the extract was dried (Na₂SO₄), filtered, and concentrated. The concentrate was purified by flash column chromatography (Analogix hexanes:ethyl acetate, 0-75%) to afford the title compound. MS (ESI) m/z 300.9 (M+H)⁺; ¹H NMR (400 MHz, CDCl₃) δ 7.79 (d, J=8.29 Hz, 2H) 7.35 (d, J=7.98 Hz, 2H) 4.06-4.23 (m, 3H) 4.01 (dd, J=7.98, 6.14 Hz, 1H) 3.51 (dd, J=8.13, 6.90 Hz, 1H) 2.45 (s, 3H) 1.82-1.98 (m, 2H) 1.31 (d, J=18.72 Hz, 6H).

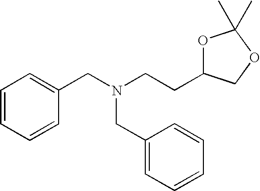

### Example 1B

### N,N-dibenzyl-2-(2,2-dimethyl-1,3-dioxolan-4-yl)ethanamine

EXAMPLE 1A (1.0 g) and dibenzylamine (0.657 mg) were placed in a microwave vial (Biotage) and dioxane (2.5 mL) was added. The vial was capped and placed in a microwave reactor (Biotage Initiator), and the mixture was heated at 150° C. for 30 minutes. The mixture was diluted with ethyl acetate and poured into water. The aqueous layer was extracted twice with ethyl acetate, and the extract was washed with brine, dried (Na₂SO₄), filtered and concentrated. The concentrate was used in the next step without further purification.

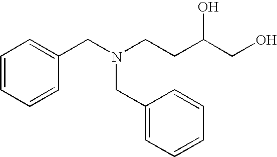

### Example 1C

### 4-(dibenzylamino)butane-1,2-diol

EXAMPLE 1B was added to tetrahydrofuran (20 mL) and 2N HCl (20 mL), and the mixture was stirred at room temperature for 30 minutes. 5N NaOH was added until the solution was basic, and the aqueous layer was extracted with chloroform. The extract was dried (MgSO₄), filtered and concentrated by rotary evaporation and the concentrate was used in the next step without further purification. MS (ESI) m/z 285.9 (M+H)⁺.

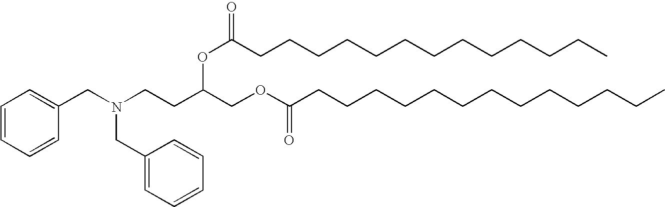

### Example 1D

### 4-(dibenzylamino)butane-1,2-diyl ditetradecanoate

A mixture of EXAMPLE 1C (700 mg), tetradecanoic acid (1.68 g), N¹-((ethylimino)methylene)-N³,N³-dimethylpropane-1,3-diamine hydrochloride (1.41 g) and 4-(dimethylamino)pyridine (45 mg) in dichloromethane (5 mL) was heated at 40° C. until the mixture was homogenous and then was stirred overnight at room temperature. Water was added along with some brine and the aqueous layer was extracted with dichloromethane (3×). The extract was dried (Na₂SO₄), filtered and the filtrate was concentrated. The concentrate was purified by flash column chromatography (Analogix 280, 0-50% ethyl acetate/hexanes) to provide the title compound. MS (ESI) m/z 706.5 (M+H)⁺; ¹H NMR (300 MHz, CDCl₃) δ 7.16-7.39 (m, 10H) 5.06-5.21 (m, 1H) 4.12 (dd, J=11.70, 3.37 Hz, 1H) 3.91 (dd, J=11.90, 5.95 Hz, 1H) 3.41-3.62 (m, 4H) 2.35-2.57 (m, 2H) 2.25 (t, J=7.54 Hz, 2H) 2.02-2.19 (m, 2H) 1.77 (q, J=7.40 Hz, 2H) 1.45-1.63 (m, 4H) 1.17-1.36 (m, 40H) 0.82-0.94 (m, 6H).

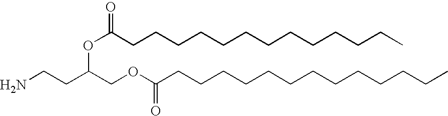

### Example 1E

### 4-aminobutane-1,2-diyl ditetradecanoate

EXAMPLE 1D (500 mg) was added to methanol/dichloromethane/ethyl acetate (1/1/1, 10 mL) and combined with catalytic Pd/C (10%). Hydrogen was introduced via a balloon, and the mixture was stirred overnight then filtered through Celite®. The filtrate was concentrated and the concentrate was used in the next step without further purification. MS (ESI) m/z 526.6 (M+H)⁺; ¹H NMR (300 MHz, CDCl₃) δ ppm 5.13-5.25 (m, 1H) 4.02-4.35 (m, 2H) 2.91-3.23 (m, 2H) 2.24-2.42 (m, 4H) 1.97-2.23 (m, 2H) 1.44-1.73 (m, 6H) 1.26 (s, 40H) 0.81-0.96 (m, 6H).

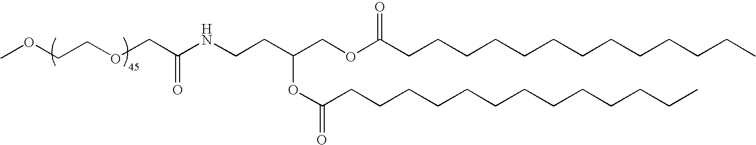

### Example 1F

### 6-oxo-2-(tetradecanoyloxy)-8,11,14,17,20,23,26,29,32,35,38,41,44,47,50,53,56,59,62,65,68,71,74,77,80,83,86,89,92, 95,98,101,104,107,110,113,116,119,122,125,128,131,134,137,140,143-hexatetracontaoxa-5-a zatetratetracontahect-1-yl myristate

mPEG2000-SCM (139 mg, Laysan Bio, Inc) and EXAMPLE 1E (100 mg) were combined in a 4 mL vial with dichloromethane (1 mL) and triethylamine (26.5 μL). The mixture was stirred at room temperature overnight. The mixture was loaded directly onto a silica gel column (Analogix) and eluted with dichlormethane/methanol (0-20%). MS (MALDI) m/z 2690.5; ¹H NMR (300 MHz, CDCl₃) 5.07-5.20 (m, 1H) 4.24 (dd, J=11.90, 3.17 Hz, 1H) 4.06 (dd, J=11.90, 6.35 Hz, 1H) 3.98 (s, 2H) 3.85-3.91 (m, 1H) 3.61-3.70 (m, 29H) 3.39-3.59 (m, 6H) 3.38 (s, 3H) 3.14-3.30 (m, 1H) 2.25-2.36 (m, 4H) 1.53-1.87 (m, 6H) 1.26 (s, 40H) 0.83-0.93 (m, 6H).

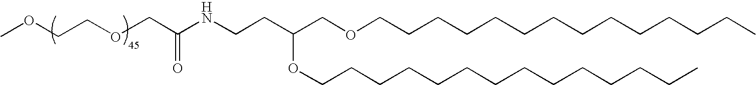

### Example 2

### N-[3,4-bis(tetradecyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanon atriacontahectan-139-amide

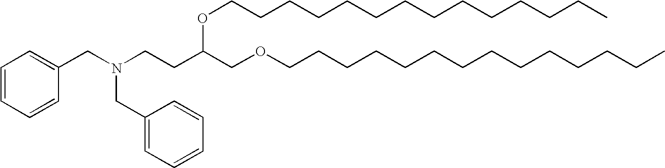

### Example 2A

### N,N-dibenzyl-3,4-bis(tetradecyloxy)butan-1-amine

EXAMPLE 1C (1 g) in toluene (6 mL) and added to NaH (0.336 g, dry, 95%) in toluene (6 mL). The mixture was stirred at room temperature for 1 hour. Tetradecyl methanesulfonate (2.15 g) was added. The mixture was heated to 90° C. overnight. The mixture was cooled to room temperature and ethanol was added followed by water until the excess NaH was destroyed. The mixture was poured into water and brine and extracted with ethyl acetate. The water was extracted with ethyl acetate, and the extract was dried (Na₂SO₄), filtered and concentrated. The concentrate was purified by an Analogix system (hexane:ethyl acetate, 0-50%)). MS (ESI) m/z 678.6 (M+H)⁺; ¹H NMR (300 MHz, CDCl₃) δ ppm 7.16-7.40 (m, 10H) 3.14-3.63 (m, 11H) 2.44-2.59 (m, 2H) 1.59-1.82 (m, 2H) 1.35-1.53 (m, 4H) 1.14-1.34 (m, 44H) 0.82-0.94 (m, 6H).

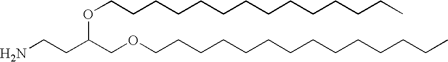

### Example 2B

### 3,4-bis(tetradecyloxy)butan-1-amine

EXAMPLE 2B was prepared using the procedure described for EXAMPLE 1E, substituting EXAMPLE 2A for EXAMPLE 1D. MS (ESI) m/z 498.5 (M+H)⁺; ¹H NMR (300 MHz, CDCl₃) δ ppm 8.24 (s, 2H) 3.53-3.70 (m, 1H) 3.34-3.53 (m, 6H) 3.07-3.34 (m, 2H) 1.87-2.13 (m, 2H) 1.48-1.67 (m, 4H) 1.16-1.39 (m, 44H) 0.82-0.94 (m, 6H).

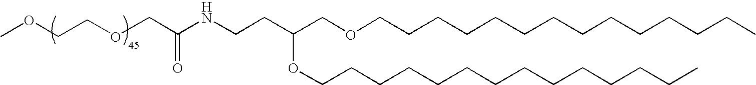

### Example 2C

### N-[3,4-bis(tetradecyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanon atriacontahectan-139-amide

EXAMPLE 2C was prepared using the procedure described for EXAMPLE 1F, substituting EXAMPLE 2B for EXAMPLE 1E. MS (MALDI) m/z 2617.6; ¹H NMR (300 MHz, CDCl₃) δ 3.95-4.02 (m, 2H) 3.83-3.92 (m, 1H) 3.68-3.72 (m, 1H) 3.65 (m, 180H) 3.35-3.60 (m, 10H) 1.59-1.73 (m, 2H) 1.49-1.60 (m, 4H) 1.18-1.36 (m, 44H) 0.82-0.94 (m, 6H).

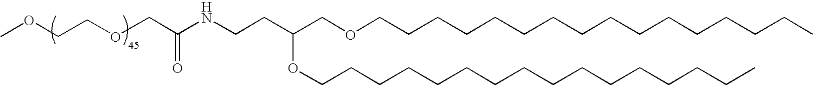

### Example 3

### N-[3,4-bis(hexadecyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanona triacontahectan-139-amide

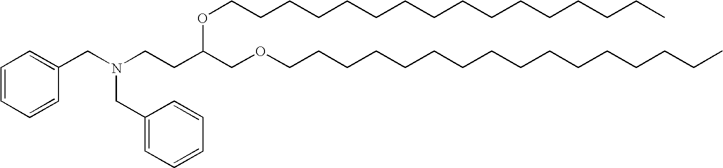

### Example 3A

### N,N-dibenzyl-3,4-bis(hexadecyloxy)butan-1-amine

EXAMPLE 3A was prepared using the procedure described for EXAMPLE 2A, substituting hexadecyl methanesulfonate for tetradecyl methanesulfonate. MS (ESI) m/z 734.6 (M+H)⁺; ¹H NMR (300 MHz, CDCl₃) δ ppm 7.15-7.41 (m, 10H) 3.12-3.64 (m, 11H) 2.41-2.64 (m, 2H) 1.35-1.80 (m, 6H) 1.15-1.34 (m, 52H) 0.81-0.94 (m, 6H).

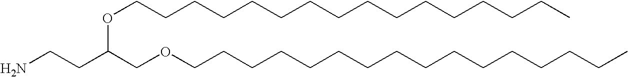

### Example 3B

### 3,4-bis(hexadecyloxy)butan-1-amine

EXAMPLE 3B was prepared using the procedure described for EXAMPLE 1E, substituting EXAMPLE 3A for EXAMPLE 2D. MS (ESI) m/z 554.6 (M+H)⁺; ¹H NMR (300 MHz, CDCl₃) δ ppm 8.12-8.38 (m, 2H) 3.54-3.70 (m, 1H) 3.33-3.53 (m, 6H) 3.06-3.33 (m, 2H) 1.84-2.14 (m, 2H) 1.46-1.71 (m, 4H) 1.14-1.37 (m, 52H) 0.81-0.94 (m, 6H).

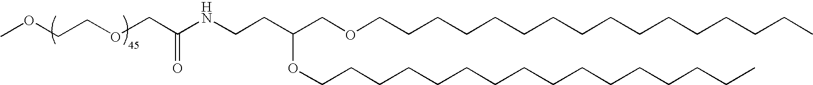

### Example 3C

### N-[3,4-bis(hexadecyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanona triacontahectan-139-amide

EXAMPLE 3C was prepared using the procedure described for EXAMPLE 1F, substituting EXAMPLE 3B for EXAMPLE 1E. MS (MALDI) m/z 2866.7; ¹H NMR (300 MHz, CDCl₃) δ ppm 3.98 (s, 2H) 3.84-3.91 (m, 1H) 3.60-3.68 (m, 180H) 3.36-3.60 (m, 11H) 1.50-1.72 (m, 6H) 1.26 (s, 52H) 0.84-0.92 (m, 6H).

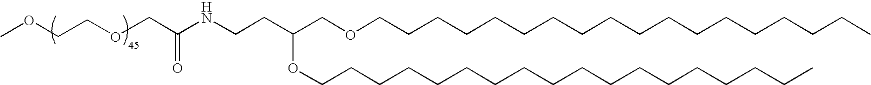

### Example 4

### N-[3,4-bis(octadecyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanona triacontahectan-139-amide

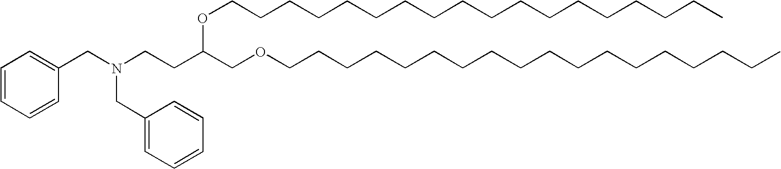

### Example 4A

### N,N-dibenzyl-3,4-bis(octadecyloxy)butan-1-amine

EXAMPLE 4A was prepared using the same procedure described for EXAMPLE 2A, substituting octadecyl methanesulfonate for tetradecyl methanesulfonate. LCMS (APCI) m/z 790.6; ¹H NMR (300 MHz, CDCl₃) δ ppm 7.15-7.41 (m, 10H) 3.10-3.68 (m, 11H) 2.39-2.68 (m, 2H) 1.35-1.80 (m, 6H) 1.14-1.34 (m, 60H) 0.81-0.94 (m, 6H).

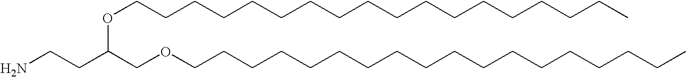

### Example 4B

### 3,4-bis(octadecyloxy)butan-1-amine

EXAMPLE 4B was prepared using the same procedure described for EXAMPLE 1E, substituting EXAMPLE 4A for EXAMPLE 1D. LCMS (APCI) m/z 610.9; ¹H NMR (300 MHz, CDCl₃) δ 3.08-3.70 (m, 9H) 1.85-2.15 (m, 2H) 1.55 (s, 4H) 1.15-1.37 (m, 60H) 0.84-0.92 (m, 6H).

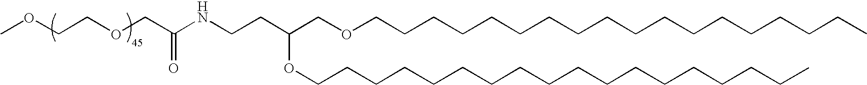

### Example 4C

### N-[3,4-bis(octadecyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanona triacontahectan-139-amide

EXAMPLE 4C was prepared using the same procedure described for EXAMPLE 1F, substituting EXAMPLE 4B for EXAMPLE 1E. MS (MALDI) m/z 2773.6; ¹H NMR (300 MHz, CDCl₃) δ ppm 3.95-4.01 (m, 2H) 3.84-3.91 (m, 1H) 3.59-3.70 (m, 180H) 3.27-3.59 (m, 11H) 1.49-1.86 (m, 6H) 1.18-1.35 (m, 60H) 0.80-0.94 (m, 6H).

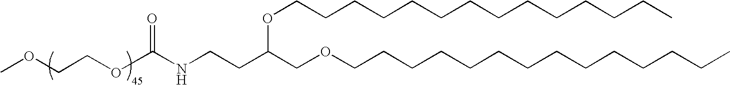

### Example 5

### 3,7,11,15,19,23,27,31,35,39,43,47,51,55,59,63,67,71,75,79,83,87,91,95,99,103,107,111,115, 119,123,127,131,135,139,143,147,151,155,159,163,167,171,175,179,182-hexatetracontaoxatrioctacontahect-1-yl 3,4-bis(tetradecyloxy)butylcarbamate

EXAMPLE 2B (100 mg) was dissolved in dichloromethane (1-2 mL) and mPEG-NPC (26.0 mg) was added. Hunig's base (26 mg) was added, and the mixture was stirred overnight at room temperature. The mixture was loaded directly onto a silica gel column (4 g Analogix) and chromatographed (Analogix 280, dichloromethane/methanol, 0-20%) to give EXAMPLE 5. MS (MALDI) m/z 2472.2; ¹H NMR (300 MHz, CDCl₃) δ 4.16-4.24 (m, 2H) 3.78-3.92 (m, 1H) 3.59-3.70 (m, 180H) 3.52-3.61 (m, 4H) 3.19-3.49 (m, 9H) 1.48-1.82 (m, 6H) 1.21-1.35 (m, 44H) 0.82-0.93 (m, 6H).

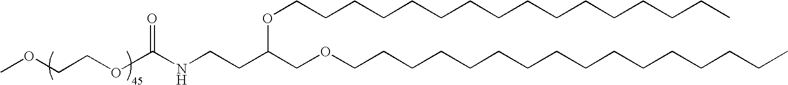

### Example 6

### 3,6,9,12,15,18,21,24,27,30,33,36,39,42,45,48,51,54,57,60,63,66,69,72,75,78,81,84,87,90, 93,96,99,102,105,108,111,114,117,120,123,126,129,132,135,138-hexatetracontaoxanonatriacontahect-1-yl 3,4-bis(hexadecyloxy)butylcarbamate

EXAMPLE 6 was prepared using the same procedure described for EXAMPLE 5, substituting EXAMPLE 3B for EXAMPLE 2B. MS (MALDI) m/z 2395.0; ¹H NMR (300 MHz, CDCl₃) δ 4.15-4.23 (m, 2H) 3.81-3.92 (m, 1H) 3.60-3.71 (m, 180H) 3.47-3.59 (m, 4H) 3.33-3.48 (m, 9H) 1.48-1.81 (m, 6H) 1.19-1.34 (m, 52H) 0.83-0.92 (m, 6H).

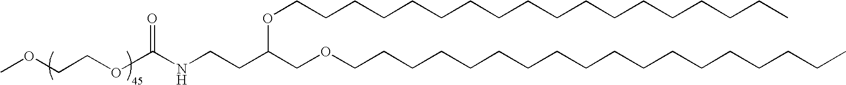

### Example 7

### 3,6,9,12,15,18,21,24,27,30,33,36,39,42,45,48,51,54,57,60,63,66,69,72,75,78,81,84,87,90, 93,96,99,102,105,108,111,114,117,120,123,126,129,132,135,138-hexatetracontaoxanonatriacontahect-1-yl 3,4-bis(octadecyloxy)butylcarbamate

EXAMPLE 7 was prepared using the same procedure described for EXAMPLE 5, substituting EXAMPLE 4B for EXAMPLE 2B. MS (MALDI) m/z 2495.8; ¹H NMR (300 MHz, CDCl₃) δ 4.16-4.24 (m, 2H) 3.82-3.92 (m, 1H) 3.60-3.71 (m, 180H) 3.49-3.59 (m, 4H) 3.17-3.49 (m, 9H) 1.48-1.80 (m, 6H) 1.18-1.37 (m, 60H) 0.82-0.93 (m, 6H).

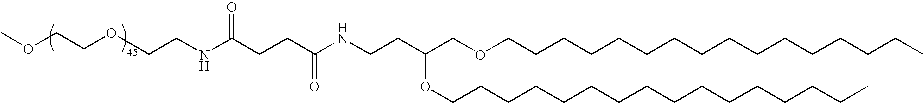

### Example 8

### N-[3,4-bis(hexadecyloxy)butyl]-N′-3,6,9,12,15,18,21,24,27,30,33,36,39,42,45,48,51,54,57,60,63,66,69,72,75,78,81,84,87,90, 93,96,99,102,105,108,111,114,117,120,123,126,129,132,135,138-hexatetracontaoxanonatriacontahect-1-ylsuccinamide

EXAMPLE 8 was prepared using the same procedure described for EXAMPLE 1F, substituting RAPP 12 2000-35 (Rapp Polymere) for mPEG2000-SCM. MS (MALDI) m/z 2584.3; ¹H NMR (300 MHz, CDCl₃) δ 6.43-6.61 (m, 2H) 3.60-3.68 (m, 200H) 3.36-3.58 (m, 16H) 2.42-2.57 (m, 4H) 1.49-1.85 (m, 6H) 1.19-1.35 (m, 52H) 0.82-0.92 (m, 6H).

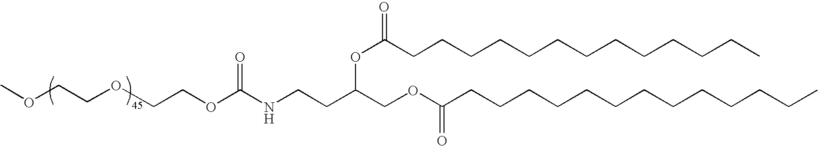

### Example 9

### 6-oxo-2-(tetradecanoyloxy)-7,10,13,16,19,22,25,28,31,34,37,40,43,46,49,52,55,58,61,64,67,70,73,76,79,82,85,88,91, 94,97,100,103,106,109,112,115,118,121,124,127,130,133,136,139,142,145-heptatetracontaox a-5-azahexatetracontahect-1-yl myristate

EXAMPLE 9 was prepared using the same procedure described for EXAMPLE 1F, substituting mPEG-NPC (Creative PEGWorks) for mPEG2000-SCM (Laysan Bio, Inc.). MS (MALDI) m/z 2588.5; ¹H NMR (300 MHz, CDCl₃) δ 5.14 (m, 1H) 4.17-4.26 (m, 3H) 4.01-4.11 (m, 1H) 3.83-3.91 (m, 1H) 3.60-3.71 (m, 180H) 3.48-3.60 (m, 4H) 3.35-3.44 (m, 5H) 2.23-2.37 (m, 4H) 1.62-1.86 (m, 6H) 1.21-1.37 (m, 40H) 0.83-0.93 (m, 6H).

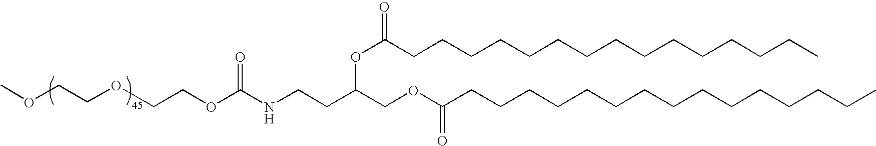

### Example 10

### 6-oxo-2-(palmitoyloxy)-7,10,13,16,19,22,25,28,31,34,37,40,43,46,49,52,55,58,61,64,67,70,73,76,79,82,85,88,91, 94,97,100,103,106,109,112,115,118,121,124,127,130,133,136,139,142,145-heptatetracontaoxa-5-azahexatetracontahect-1-yl palmitate

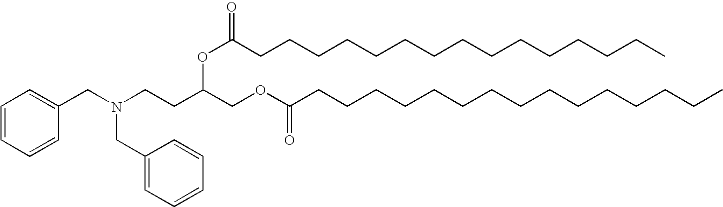

### Example 10A

### 4-(dibenzylamino)butane-1,2-diyldipalmitate

EXAMPLE 10A was prepared using the same procedure described for EXAMPLE 1D, substituting hexadecanoic acid for tetradecanoic acid. MS (ESI) m/z 762.4 (M+H)⁺; ¹H NMR (300 MHz, CDCl₃) δ ppm 7.15-7.42 (m, 10H) 5.06-5.21 (m, 1H) 4.12 (dd, J=11.90, 3.57 Hz, 1H) 3.91 (dd, J=11.90, 5.95 Hz, 1H) 3.43-3.62 (m, 4H) 2.34-2.58 (m, 2H) 2.25 (t, J=7.34 Hz, 2H) 2.01-2.16 (m, 2H) 1.77 (q, J=7.14 Hz, 2H) 1.40-1.64 (m, 4H) 1.14-1.37 (m, 48H) 0.82-0.95 (m, 6H).

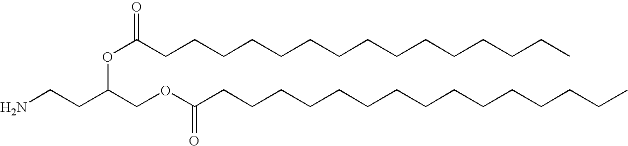

### Example 10B

### 4-aminobutane-1,2-diyldipalmitate

EXAMPLE 10B was prepared using the same procedure described for EXAMPLE 1E, substituting EXAMPLE 10A for 1D. MS (ESI) m/z 482.6 (M+H)⁺.

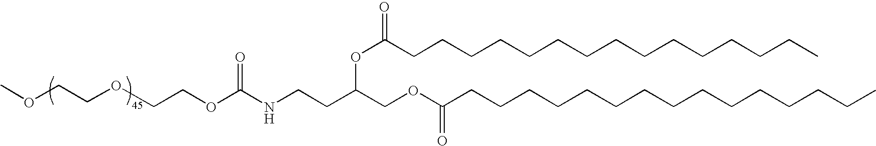

### Example 10C

### 6-oxo-2-(palmitoyloxy)-7,10,13,16,19,22,25,28,31,34,37,40,43,46,49,52,55,58,61,64,67,70,73,76,79,82,85,88,91, 94,97,100,103,106,109,112,115,118,121,124,127,130,133,136,139,142,145-heptatetracontaoxa-5-azahexatetracontahect-1-yl palmitate

EXAMPLE 10C was prepared using the same procedure described for EXAMPLE 1F, substituting EXAMPLE 10B for EXAMPLE 1E and substituting mPEG-NPC (Creative PEGWorks) for mPEG2000-SCM (Laysan Bio, Inc.). MS (MALDI) m/z 2689.0; ¹H NMR (300 MHz, CDCl₃) δ ppm 5.09-5.19 (m, 1H) 4.17-4.26 (m, 3H) 4.01-4.11 (m, 1H) 3.73-3.91 (m, 1H) 3.61-3.70 (m, 180H) 3.48-3.60 (m, 4H) 3.35-3.44 (m, 5H) 2.23-2.36 (m, 4H) 1.54-1.84 (m, 6H) 1.21-1.36 (m, 48H) 0.82-0.93 (m, 6H).

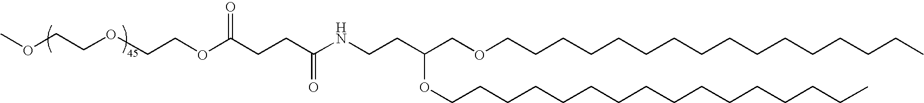

### Example 11

### 3,6,9,12,15,18,21,24,27,30,33,36,39,42,45,48,51,54,57,60,63,66,69,72,75,78,81,84,87,90, 93,96,99,102,105,108,111,114,117,120,123,126,129,132,135,138-hexatetracontaoxanonatriacontahect-1-yl 4-{[3,4-bis(hexadecyloxy)butyl]amino}-4-oxobutanoate

EXAMPLE 3B (100 mg) and mPEG-COOH (278 mg, PSA-288, Creative PEGWorks) were combined in dichloromethane (2 mL). N¹-((ethylimino)methylene)-N³,N³-dimethylpropane-1,3-diamine hydrochloride (346 mg) was added followed by 4-(dimethylamino)pyridine (2 mg). The mixture was stirred overnight at room temperature then loaded directly onto a 4 g silica gel column (Analogix) and purified (Analogix 280, dichloromethane:methanol 0-20%). (MALDI) m/z 2628.4; ¹H NMR (300 MHz, CDCl₃) δ ppm 4.19-4.28 (m, 2H) 3.83-3.92 (m, 1H) 3.65 (none, 180H) 3.36-3.59 (m, 16H) 2.69 (t, J=6.78 Hz, 2H) 2.43 (t, J=6.95 Hz, 2H) 1.47-1.71 (m, 6H) 1.22-1.32 (m, 52H) 0.84-0.92 (m, 6H).

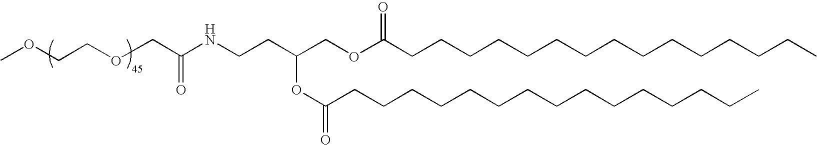

### Example 12

### 6-oxo-2-(palmitoyloxy)-8,11,14,17,20,23,26,29,32,35,38,41,44,47,50,53,56,59,62,65,68,71,74,77,80,83,86,89,92, 95,98,101,104,107,110,113,116,119,122,125,128,131,134,137,140,143-hexatetracontaoxa-5-azatetratetracontahect-1-yl palmitate

This example was prepared using the same procedure described for EXAMPLE 1F, substituting EXAMPLE 10B for EXAMPLE 1E. MS (MALDI) m/z 2835.3; ¹H NMR (300 MHz, CDCl₃) δ ppm 5.07-5.20 (m, 1H) 4.24 (dd, J=11.90, 3.57 Hz, 1H) 4.06 (dd, J=11.90, 6.35 Hz, 1H) 3.98 (s, 2H) 3.61-3.68 (m, 180H) 3.49-3.60 (m, 4H) 3.36-3.48 (m, 5H) 2.25-2.36 (m, 4H) 1.77-1.87 (m, 2H) 1.26 (m, 48H) 0.83-0.93 (m, 6H).

### Example 13

### 1-(2,3-bis((9Z,12Z)-octadeca-9,12-dienyloxy)propyl)pyrrolidine

3-(Pyrrolidin-1-yl)propane-1,2-diol (150 mg) and linoleyl methane sulfonate (1.068 g) were combined in toluene (5 mL). Sodium hydride (104 mg, 95% w/w) was added, and the mixture was stirred for 5 minutes, heated in a sealed vial at 100° C. for 2 hours, cooled to room temperature, quenched with methanol and partitioned between ethyl acetate (100 mL) and water (50 mL). The extract was dried over Na₂SO₄, filtered and concentrated. The concentrate was purified by flash chromatography on silica gel (0-5% methanol in dichloromethane). ¹H NMR (300 MHz, CDCl₃) δ 5.14-5.74 (m, 8H) 3.25-3.75 (m, 7H) 2.32-2.94 (m, 10H) 1.88-2.27 (m, 8H) 1.47-1.88 (m, 8H) 1.13-1.47 (m, 32H) 0.77-1.08 (m, 6H).

### Example 14

### 1-(3,4-bis((9Z,12Z)-octadeca-9,12-dienyloxy)butyl)pyrrolidine

### Example 14A

### 2-(2,2-dimethyl-1,3-dioxolan-4-yl)ethyl 4-methylbenzenesulfonate

2-(2,2-dimethyl-1,3-dioxolan-4-yl)ethanol (5 g) in dichloromethane (86 mL) at 0° C. was treated with TEA (6.9 g), para-toluenesulfonyl chloride chloride (6.5 g) and 4-DMAP (0.42 g), stirred overnight, quenched with saturated NH₄Cl and diluted with ethyl acetate. The extract was dried (Na₂SO₄), filtered, and concentrated. The concentrate was purified by flash column chromatography (0-100% ethyl acetate/hexanes, Analogix). ¹H NMR (400 MHz, CDCl₃) δ 7.79 (d, J=8.29 Hz, 2H) 7.35 (d, J=7.98 Hz, 2H) 4.06-4.23 (m, 3H) 4.01 (dd, J=7.98, 6.14 Hz, 1H) 3.51 (dd, J=8.13, 6.90 Hz, 1H) 2.45 (s, 3H) 1.82-1.98 (m, 2H) 1.31 (d, J=18.72 Hz, 6H).

### Example 14B

### 1-(3,4-bis((9Z,12Z)-octadeca-9,12-dienyloxy)butyl)pyrrolidine

2-(2,2-dimethyl-1,3-dioxolan-4-yl)ethyl 4-methylbenzenesulfonate (500 mg), pyrrolidine (1-2 eq) and Hunig's base (2 eq) in dioxane (2.2 mL) was microwaved (Biotage Initiator) for 15 minutes at 140° C., treated with 4NHCl (4 mL) until acidic, stirred overnight at room temperature, treated with 6N NaOH until basic, diluted with water and extracted with chloroform. The extract was dried (Na₂SO₄), filtered and concentrated. The concentrate in toluene (0.3 M) was treated with NaH (5-10 eq), stirred for 45 minutes, treated with (9Z,12Z)-octadeca-9,12-dienyl methanesulfonate (Nu-Check Prep, 2.5 eq), stirred at 80-90° C. for 4 hours, treated with ethanol then ethyl acetate and water. The water was extracted with ethyl acetate, and the extract was dried (Na₂SO₄), filtered and concentrated. The concentrate was purified by flash column chromatography (0-100% ethyl acetate/hexanes, Analogix) to afford the title compound. ¹H NMR (300 MHz, CDCl₃) δ 5.25-5.45 (m, 8H) 3.51-3.63 (m, 1H) 3.33-3.51 (m, 6H) 2.77 (t, J=6.10 Hz, 4H) 2.44-2.58 (m, 6H) 2.05 (q, J=6.55 Hz, 8H) 1.48-1.82 (m, 10H) 1.20-1.44 (m, 34H) 0.84-0.96 (m, 6H).

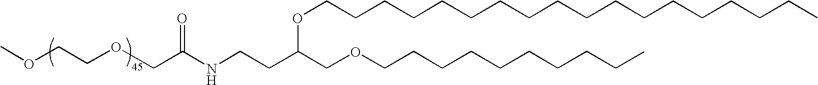

### Example 15

### N-[4-(decyloxy)-3-(octadecyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide

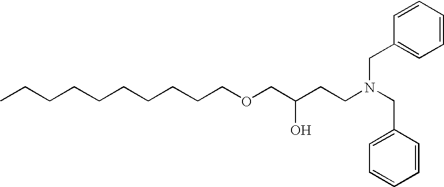

### 1-(decyloxy)-4-(dibenzylamino)butan-2-ol

### Example 15A

Into a 100 mL round-bottomed flask was added EXAMPLE 1C (1 g, 3.50 mmol) and the mixture was dissolved in tetrahydrofuran (11.68 ml), followed by NaH (0.252 g, 10.51 mmol) to give a suspension. The solution was stirred at room temperature for 30 minutes. 1-Bromodecane (1.598 ml, 7.71 mmol) was added at room temperature, then the mixture was warmed to 60° C. for 12 hours. The reaction was diluted with N,N-dimethylformamide and heated to 90° C. overnight. The reaction was cooled to room temperature, and quenched with water. The reaction was poured into ethyl acetate, and the resulting layers were separated. The organics were collected, dried over MgSO₄, filtered, and reduced in vacuo. The residue was purified via an Analogix flash chromatography system (hexanes:ethyl acetate) to afford the title compound. LC/MS m/z 426 (M+H)⁺.

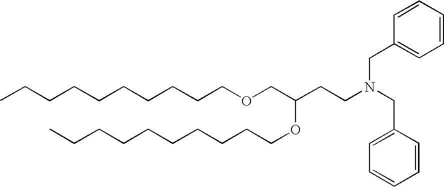

### N,N-dibenzyl-3,4-bis(decyloxy)butan-1-amine

### Example 15B

Into a 100 mL round-bottomed flask was added EXAMPLE 1C (1 g, 3.50 mmol) and the mixture was dissolved in tetrahydrofuran (11.68 ml), followed by NaH (0.252 g, 10.51 mmol) to give a suspension. The solution was stirred at room temperature for 30 minutes. 1-Bromodecane (1.598 ml, 7.71 mmol) was added at room temperature, then the mixture was warmed to 60° C. for 12 hours. The reaction was diluted with N,N-dimethylformamide and heated to 90° C. overnight. The reaction was cooled to room temperature, and quenched with water. The reaction was poured into ethyl acetate, and the resulting layers were separated. The organics were collected, dried over MgSO₄, filtered, and reduced in vacuo. The residue was purified via an Analogix flash chromatography system (hexanes:ethyl acetate) to afford the title compound. LC/MS m/z 566 (M+H)⁺.

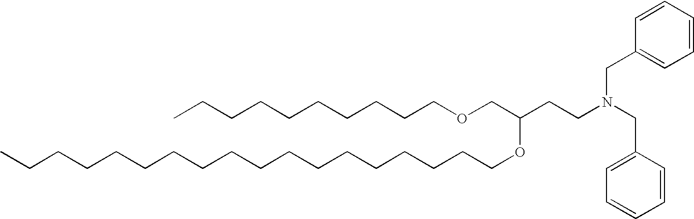

### N,N-dibenzyl-4-(decyloxy)-3-(octadecyloxy)butan-1-amine

### Example 15C

Into a 15 mL vial was added EXAMPLE 15A (0.52 g, 1.222 mmol) and NaH (0.088 g, 3.67 mmol) in N,N-dimethylformamide (6.11 ml) to give a suspension, and the reaction stirred for 15 minutes at room temperature. Octadecyl methanesulfonate (0.468 g, 1.344 mmol) was added and the reaction was heated to 90° C. overnight. The reaction was cooled to room temperature, quenched with water, and diluted with diethyl ether. The organics were separated, and the aqueous layer was extracted with diethyl ether. The organic layers were combined, dried over MgSO₄, filtered and reduced in vacuo. The residue was purified via Analogix using a gradient elution (100% to 90% Hexane/ethyl acetate) to afford the title compound. MS (ESI) m/z 678.8 (M+H)⁺.

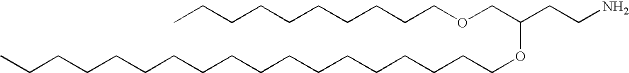

### 4-(decyloxy)-3-(octadecyloxy)butan-1-amine

### Example 15D

Into a 50 mL round-bottomed flask was added EXAMPLE 15C (0.3 g, 0.442 mmol) and Pd/C (0.047 g, 0.044 mmol) in CH₂Cl₂ (2.212 ml)/methanol (2.212 ml) to give a black suspension, the system was purged via vacuum, then 1 atm H₂. The process was repeated 3 times. The reaction was stirred at room temperature under 1 atm of H₂ for 18 hrs. The reaction was treated with Celite, filtered over Celite. The Celite pad was washed with CH₂Cl₂/MeOH. The organics were reduced in vacuo to afford a solid. The residue was purified via Analogix using a gradient elution (100% to 80% CH₂Cl₂/MeOH) to afford EXAMPLE 15D. MS (ESI) m/z 498.7 (M+H)⁺.

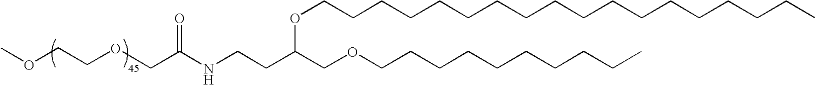

### Example 15E

### N-[4-(decyloxy)-3-(octadecyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide

EXAMPLE 15D (75 mg, 0.151 mmol) and Hunig's base (30.1 uL) were combined in dichloromethane (2 mL) at room temperature. mPEG-SCM (MW 2000, Laysan Bio, 172 mg, 0.086 mmol) was added to the solution and the mixture was stirred overnight at room temperature. The reaction mixture was loaded directly onto silica gel and purified by flash column chromatography (Analogix) (100% ethyl acetate, followed by 0-15% methanol in dichloromethane) to afford the title compound. MS (MALDI) m/z 2750.8; ¹H NMR (300 MHz, CHLOROFORM-D) δ ppm 3.98 (s, 2H) 3.85-3.90 (m, 1H) 3.61-3.72 (m, 180H) 3.36-3.60 (m, 11H) 1.25 (s, 44H) 0.83-0.93 (m, 6H).

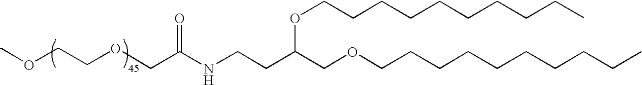

### Example 16

### N-[3,4-bis(decyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide

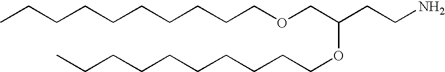

### 3,4-bis(decyloxy)butan-1-amine

### Example 16A

Into a 50 mL round-bottomed flask was added EXAMPLE 15B (0.636 g, 1.124 mmol) and Pd/C (0.239 g, 0.225 mmol) in methanol (1.873 ml)/CH₂Cl₂ (1.873 ml) to give a suspension. The reaction mixture was purged with H₂, and evacuated in vacuo. This cycle was repeated 3 times, and the mixture was allowed to stir under 1 atm of H₂ at room temperature overnight. The mixture was treated with diatomaceous earth, and filtered over diatomaceous earth. The diatomaceous earth was washed with CH₂Cl₂ and methanol. The organics were reduced in vacuo. The residue was purified via Analogix using a gradient elution (0 to 20% methanol in CH₂Cl₂) to afford the title compound. MS (ESI) m/z 386.3 (M+H)⁺.

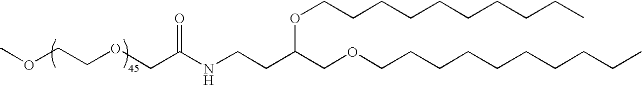

### Example 16B

### N-[3,4-bis(decyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide

Example 16B was prepared using the same procedure described for Example 1F, substituting Example 16A for Example 1E. MS (MALDI) m/z 2726.3; ¹H NMR (300 MHz, CHLOROFORM-D) δ ppm 3.98 (s, 2H) 3.87 (dd, J=5.76, 4.07 Hz, 1H) 3.61-3.68 (m, 180H) 3.36-3.59 (m, 11H) 1.50-1.61 (m, 6H) 1.26 (s, 28H) 0.83-0.93 (m, 6H).

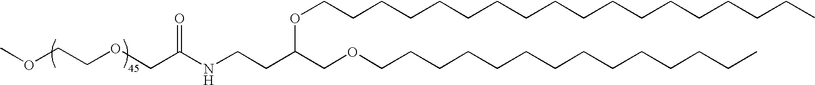

### Example 17

### N-[3-(octadecyloxy)-4-(tetradecyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide

Example 17 was prepared using the same procedure described for Example 15, substituting 1-bromotetradecane for 1-bromodecane in Example 15A. MS (MALDI) m/z 2895.9; ¹H NMR (300 MHz, CHLOROFORM-D) δ ppm 3.98 (s, 2H) 3.84-3.92 (m, 1H) 3.62-3.68 (m, 180H) 3.35-3.60 (m, 11H) 1.46-1.57 (m, 6H) 1.25 (s, 52H) 0.83-0.92 (m, 6H).

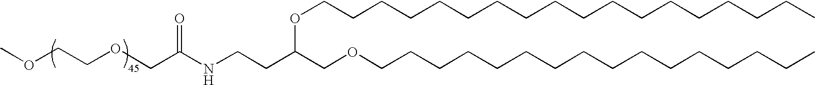

### Example 18

### N-[4-(hexadecyloxy)-3-(octadecyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide

Example 18 was prepared using the same procedure described for Example 15, substituting 1-bromohexadecane for 1-bromodecane in Example 15A. MS (MALDI) m/z 2878.5; ¹H NMR (300 MHz, CHLOROFORM-D) δ ppm 3.98 (s, 2H) 3.84-3.91 (m, 1 H) 3.62-3.67 (m, 180H) 3.34-3.60 (m, 11H) 1.54 (d, J=7.46 Hz, 6H) 1.21-1.35 (m, 56H) 0.84-0.91 (m, 6H).

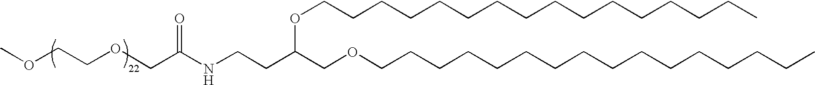

### Example 19

### N-[3,4-bis(hexadecyloxy)butyl]-2,5,8,11,14,17,20,23,26,29,32,35,38,41,44,47,50,53,56,59,62,65,68-tricosaoxaheptacontan-70-amide

Example 19 was prepared using the same procedure described for Example 1F, substituting mPEG1000-SCM (Laysan Bio, Inc.) for mPEG2000-SCM (Laysan Bio, Inc.) and Example 3B for Example 1E. MS (MALDI) m/z 1794.3; ¹H NMR (300 MHz, CHLOROFORM-D) δ ppm 3.98 (s, 2H) 3.82-3.91 (m, 1H) 3.62-3.68 (m, 88H) 3.35-3.61 (m, 11H) 1.48-1.60 (m, 6H) 1.20-1.36 (m, 52H) 0.83-0.93 (m, 6H).

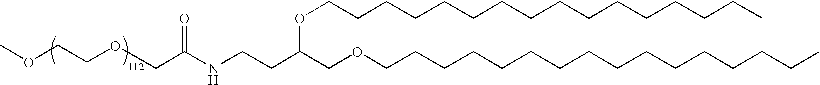

### Example 20

### N-[3,4-bis(hexadecyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137,140,143,146,149,152,155, 158,161,164,167,170,173,176,179,182,185,188,191,194,197,200,203,206,209,212,215, 218,221,224,227,230,233,236,239,242,245,248,251,254,257,260,263,266,269,272,275,278, 281,284,287,290,293,296,299,302,305,308,311,314,317,320,323,326,329,332,335,338-113 oxa340n-340-amide

Example 20 was prepared using the same procedure described for Example 1F, substituting mPEG5000-SCM (Laysan Bio, Inc.) for mPEG2000-SCM (Laysan Bio, Inc.) and Example 3B for Example 1E. MS (MALDI) m/z 5978.3; ¹H NMR (300 MHz, CHLOROFORM-D) δ ppm 3.98 (s, 2H) 3.87 (dd, J=5.93, 4.24 Hz, 1H) 3.61-3.68 (m, 448H) 3.35-3.60 (m, 11H) 1.46-1.62 (m, 6H) 1.25 (s, 52H) 0.82-0.92 (m, 6H).

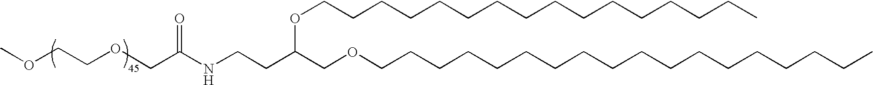

### Example 21

### N-[3-(hexadecyloxy)-4-(octadecyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide

Example 21 was prepared using the same procedure described for Example 15, substituting 1-bromooctadecane for 1-bromodecane in Example 15A and hexadecyl methanesulfonate for octadecyl methanesulfonate in Example 15B. MS (MALDI) m/z 2746.3; ¹H NMR (300 MHz, CHLOROFORM-D) δ ppm 3.98 (s, 2H) 3.87 (dd, J=5.76, 4.07 Hz, 1H) 3.60-3.69 (m, 180H) 3.36-3.61 (m, 11H) 1.55 (s, 6H) 1.25 (s, 56H) 0.83-0.93 (m, 6H).

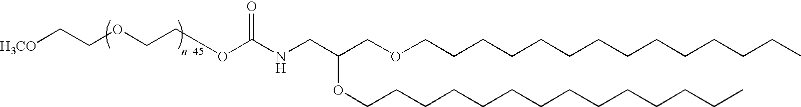

### Example 22

### N-(2,3-dimyristyloxypropyl)carbamate polyethyleneglycol-2000 methyl ether

EXAMPLE 22 was prepared using the known synthetic route; see: Heyes, J.; Hall, K.; Tailor, V.; Lenz, R.; MacLachlan, I. J. Controlled Release 2006, 112, 280-290.

### Example 23

### N,N-dimethyl-2,3-bis((9Z,12Z)-octadeca-9,12-dienyloxy)propan-1-amine

Example 23 was prepared using procedures disclosed in the following reference: J. Controlled Release 2005, 107, 276-287.

We claim:

1. A polyethylene glycol (PEG)-lipid conjugate having Formula I 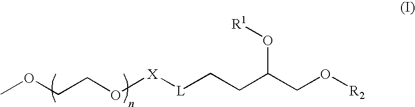
wherein
R¹ and R² are independently R³, or C(O)R³; or
R¹ and R² together are C(R³)₂;
R³ is C₈-C₂₄ alkyl;
L is C(OCH₃)₂, NHC(O), C(O)NH, OC(O)NH, NHC(O)O, NHC(O)NH, N(N)C(O), C(O)N(N), SS, NHC(O)L²C(O)O, NHC(O)L²C(O)NH, OC(O)L²C(O)O, OC(O)L²C(O)NH, C(O)O, OC(O), S, O, CH₂CH(═N)NHR⁴C(O), or C(═NNHCH₃)R⁴;
R⁴ is aryl or heteroaryl;
L² is C₁-C₆ alkyl;
X is a bond or C₁-C₆ alkyl; and
n is 10-200.

2. A Cationic-Based Lipid Encapsulation System (CaBLES) comprising:
one or more (PEG)-lipid conjugates of claim 1,
one or more non-cationic lipids, and
one or more a cationic lipids.

3. A Lipid-Based Particle, comprising:
one or more (PEG)-lipid conjugates of claim 1,
one or more non-cationic lipids,
one or more cationic lipids, and
one or more a therapeutic agents.

4. The CaBLES of claim 2, or the Lipid-Based Particle of claim 3, wherein the PEG-lipid conjugate comprises 0.1 to about 20 weight/weight percent of total lipid in the particle.

5. The CaBLES of claim 2, or the Lipid-Based Particle of claim 3, wherein one or more non-cationic lipids is chosen from cholesterol, cholesterol sulfate, ceramide, sphingomyelin, lecithin, sphingomyelin, egg sphingomyelin, milk sphingomyelin; egg phosphatidylcholine, hydrogenated egg phosphatidylcholine, hydrogenated soybean phosphatidylethanolamine, egg phosphatidylethanolamine, hydrogenated soybean phosphatidylcholine, soybean phosphatidylcholine, 1,2-dilauroyl-sn-glycerol, 1,2-dimyristoyl-sn-glycerol, 1,2-dipalmitoyl-sn-glycerol, 1,2-distearoyl-sn-glycerol, 1,2-dilauroyl-sn-glycero-3-phosphatidic acid, 1,2-dimyristoyl-sn-glycero-3-phosphatidic acid, 1,2-dipalmitoyl-sn-glycero-3-phosphatidic acid, 1,2-distearoyl-sn-glycero-3-phosphatidic acid, 1,2-diarachidoyl-sn-glycero-3-phosphocholine, 1,2-dilauroyl-sn-glycero-3-phosphocholine, 1,2-dimyristoyl-sn-glycero-3-phosphocholine, dioleoylphosphatidylcholine, 1,2-dierucoyl-sn-glycero-3-phosphocholine, 1-myristoyl-2-palmitoyl-sn-glycero-3-phosphocholine, 1-myristoyl-2-stearoyl-sn-glycero-3-phosphocholine, 1-palmitoyl-2-myristoyl-sn-glycero-3-phosphocholine, 1-palmitoyl-2-stearoyl-sn-glycero-3-phosphocholine, 1-stearoyl-2-myristoyl-sn-glycero-3-phosphocholine, 1-stearoyl-2-palmitoyl-sn-glycero-3-phosphocholine, 1-myristoyl-2-oleoyl-sn-glycero-3-phosphocholine, 1-palmitoyl-2-oleoyl-sn-glycero-3-phosphocholine; 1-stearoyl-2-oleoyl-sn-glycero-3-phosphocholine, 1-myristoyl-2-lyso-sn-glycero-3-phosphocholine, 1-palmitoyl-2-lyso-sn-glycero-3-phosphocholine, 1-stearoyl-2-lyso-sn-glycero-3-phosphocholine, 1,2-dipalmitoyl-sn-glycero-O-ethyl-3-phosphocholine, 1,2-dipalmitoyl-sn-glycero-3-phosphocholine; 1,2-distearoyl-sn-glycero-3-phosphocholine; 1-palmitoyl-2-linoleoyl-sn-glycero-3-phosphocholine, dioleoylphosphatidylethanolamine, palmitoyloleoyl-phosphatidylethanolamine, dioleoylphosphatidylglycerol, 1,2-dilauroyl-sn-glycero-3-phosphoethanolamine, 1,2-dimyristoyl-sn-glycero-3-phosphoethanolamine, 1,2-dipalmitoyl-sn-glycero-3-phosphoethanolamine, 1,2-distearoyl-sn-glycero-3-phosphoethanolamine, 1,2-dioleoyl-sn-glycero-3-phosphoethanolamine, 1,2-dilauroyl-sn-glycero-3-phosphoglycerol, 1,2-dimyristoyl-sn-glycero-3-phosphoglycerol, 1,2-dimyristoyl-sn-glycero-3-phospho-sn-1-glycerol, 1,2-dipalmitoyl-sn-glycero-3-phosphoglycerol, 1,2-distearoyl-sn-glycero-3-phosphoglycero, 1,2-distearoyl-sn-glycero-3-phospho-sn-1-glycerol, 1-palmitoyl-2-oleoyl-sn-glycero-3-phosphoglycerol, 1-palmitoyl-2-oleoyl-sn-glycero-3-phosphoglycerol, 1,2-dipalmitoyl-sn-glycero-3-phospho-L-serine, 1,2-dimyristoyl-sn-glycero-3-phospho-L-serine, 1,2-dipalmitoyl-sn-glycero-3-phospho-L-serine, 1,2-distearoyl-sn-glycero-3-phospho-L-serine, 1,2-dioleoyl-sn-glycero-3-phospho-L-serine, 1-palmitoyl-2-oleoyl-sn-glycero-3-phospho-L-serine or a mixture thereof.

6. The CaBLES of claim 2, or the Lipid-Based Particle of claim 3, wherein the non-cationic lipid comprises about 5 to about 90 weight/weight percent of total lipid in the particle.

7. The CaBLES of claim 2, or the Lipid-Based Particle of claim 3, wherein the cationic lipid is N,N-dioleyl-N,N-dimethylammonium chloride, DC-Chol; 1,3-dioleoyloxy-2-(6-carboxyspermyl)-propyl amide, dioctadecylamidoglycyl spermine, N,N-distearyl-N,N-dimethylammonium bromide, N-(2,3-dioleyloxy)propyl)-N,N-dimethylammonium chloride, 1,2-dioleoyl-3-trimethylammonium-propane chloride, 1,2-dilineoyl-3-dimethylammonium-propane, N-(1-(2,3-dioleyloxy)propyl)-N,N,N-trimethylammonium chloride, 1,2-dioleoyl-3-dimethylammonium propane, 1,2-distearyloxy-N,N-dimethyl-3-aminopropane; didodecyldimethylammonium bromide, dioleoyloxy-N-(2-sperminecarboxamido)ethyl)-N,N-dimethyl-1-propanaminiumtrifluoroacetate, 1,2-dimyristyloxypropyl-3-dimethyl-hydroxyethyl ammonium bromide, 1,2-dioleoylcarbamyl-3-dimethylammoniumpropane, tetramethyltetrapalmitoyl spermine, tetramethyltetraoleyl spermine, tetramethyldioleyl spermine, tetramethyltetramyristyl spermine, tetramethyltetralauryl spermine, 1-(2-((9Z,12Z)-octadeca-9,12-dienyloxy)-1-(((9Z,12Z)-octadeca-9,12-dienyloxy)methyl)ethyl)pyrrolidine; N,N-dimethyl-N-(2-((9Z,12Z)-octadeca-9,12-dienyloxy)-1-(((9Z,12Z)-octadeca-9,12-dienyloxy)methyl)ethyl)amine; N-(3-(1H-imidazol-1-yl)propyl)-N-(2((9Z,12Z)-octadeca-9,12-dienyloxy)-1-(((9Z,12Z)-octadeca-9,12-dienyloxy)methyl)ethyl)amine; 1-methyl-4-(2((9Z,12Z)-octadeca-9,12-dienyloxy)-1-(((9Z,12Z)-octadeca-9,12-dienyloxy)methyl)ethyl)piperazine; 4-(2-((9Z,12Z)-octadeca-9,12-dienyloxy)-1-(((9Z,12Z)-octadeca-9,12-dienyloxy)methyl)ethyl)morpholine; N-(2-((9Z,12Z)-octadeca-9,12-dienyloxy)-1-(((9Z,12Z)-octadeca-9,12-dienyloxy)methyl)ethyl)-N-(3-pyrrolidin-1-ylpropyl)amine; N,N-dimethyl-N′-(2-((9Z,12Z)-octadeca-9,12-dienyloxy)-1-(((9Z,12Z)-octadeca-9,12-dienyloxy)methyl)ethyl)ethane-1,2-diamine; N-(2-(4-methylpiperazin-1-yl)ethyl)-N-(2-((9Z,12Z)-octadeca-9,12-dienyloxy)-1-(((9Z,12Z)-octadeca-9,12-dienyloxy)methyl)ethyl)amine; N-(2-(1H-imidazol-4-yl)ethyl)-N-(2-((9Z,12Z)-octadeca-9,12-dienyloxy)-1-(((9Z,12Z)-octadeca-9,12-dienyloxy)methyl)ethyl)amine; N,N-dimethyl-N-(3-(4-(2-((9Z,12Z)-octadeca-9,12-dienyloxy)-1-(((9Z,12Z)-octadeca-9,12-dienyloxy)methyl)ethyl)piperazin-1-yl)propyl)amine; 1,3-bis((9Z,12Z)-octadeca-9,12-dienyloxy)propan-2-amine; N-((1-methylpiperidin-4-yl)methyl)-N-(2((9Z,12Z)-octadeca-9,12-dienyloxy)-1-(((9Z,12Z)-octadeca-9,12-dienyloxy)methyl)ethyl)amine; N-(2-((9Z,12Z)-octadeca-9,12-dienyloxy)-1-(((9Z,12Z)-octadeca-9,12-dienyloxy)methyl)ethyl)-N-(3-(pyrrolidin-1-ylmethyl)benzyl)amine; N-methyl-N-(2-((9Z,12Z)-octadeca-9,12-dienyloxy)-1-(((9Z,12Z)-octadeca-9,12\dienyloxy)methyl)ethyl)-N-(3-pyrrolidin-1-ylpropyl)amine; N-(3-((4-methylpiperazin-1-yl)methyl)benzyl)-N-(2((9Z,12Z)-octadeca-9,12-dienyloxy)-1-(((9Z,12Z)-octadeca-9,12-dienyloxy)methyl)ethyl)amine; N-methyl-N-((1-methylpiperidin-4-yl)methyl)-N-(2((9Z,12Z)-octadeca-9,12-dienyloxy)-1-(((9Z,12Z)-octadeca-9,12-dienyloxy)methyl)ethyl)amine; N,N,N′-trimethyl-N′-(2-((9Z,12Z)-octadeca-9,12-dienyloxy)-1-(((9Z,12Z)-octadeca-9,12-dienyloxy)methyl)ethyl)propane-1,3-diamine; N-methyl-N-(2-((9Z,12Z)-octadeca-9,12-dienyloxy)-1-(((9Z,12Z)-octadeca-9,12-dienyloxy)methyl)ethyl)-N-(3-(pyrrolidin-1-ylmethyl)benzyl)amine; 1-(2-(1H-imidazol-1-yl)ethyl)-4-(2-((9Z,12Z)-octadeca-9,12-dienyloxy)-1-(((9Z,12Z)-octadeca-9,12-dienyloxy)methyl)ethyl)piperazine; N-(2-((9Z,12Z)-octadeca-9,12-dienyloxy)-1-(((9Z,12Z)-octadeca-9,12-dienyloxy)methyl)ethyl)-N-((2-pyrrolidin-1-ylpyridin-3-yl)methyl)amine; (9Z,9′Z,12Z,12′Z)-2-(4-methylpiperazin-1-yl)propane-1,3-diyl dioctadeca-9,12-dienoate; (9Z,9′Z,12Z,12′Z)-2-(3-(pyrrolidin-1-yl)propylamino)propane-1,3-diyl dioctadeca-9,12-dienoate; 1-methyl-4-(3((9Z,12Z)-octadeca-9,12-dienyloxy)-2-(((9Z,12Z)-octadeca-9,12-dienyloxy)methyl)propyl)piperazine; 1-(3-((9Z,12Z)-octadeca-9,12-dienyloxy)-2-(((9Z,12Z)-octadeca-9,12-dienyloxy)methyl)propyl)pyrrolidine; N-(3-aminopropyl)-N′-{3-[(2-[(9Z,12Z)-octadeca-9,12-dienyloxy]-1-{[(9Z,12Z)-octadeca-9,12-dienyloxy]methyl}ethyl)amino]propyl}butane-1,4-diamine; N-(3-[(9Z,12Z)-octadeca-9,12-dienyloxy]-2-{[(9Z,12Z)-octadeca-9,12-dienyloxy]methyl}propyl)-N-(3-pyrrolidin-1-ylpropyl)amine; N,N-dimethyl-N-(3-[(9Z,12Z)-octadeca-9,12-dienyloxy]-2-{[(9Z,12Z)-octadeca-9,12-dienyloxy]methyl}propyl)amine; 3-[(9Z,12Z)-octadeca-9,12-dienyloxy]-2-{[(9Z,12Z)-octadeca-9,12-dienyloxy]methyl}propyl 2-(diethylamino)ethylcarbamate; 3-[(9Z,12Z)-octadeca-9,12-dienyloxy]-2-{[(9Z,12Z)-octadeca-9,12-dienyloxy]methyl}propyl 2-pyrrolidin-1-ylethylcarbamate; 3-[(9Z,12Z)-octadeca-9,12-dienyloxy]-2-{[(9Z,12Z)-octadeca-9,12-dienyloxy]methyl}propyl 2-(dimethylamino)ethylcarbamate; 1-(2-[(9Z,12Z)-octadeca-9,12-dienyloxy]-1-{[(9Z,12Z)-octadeca-9,12-dienyloxy]methyl}ethyl)-4-(2-pyrrolidin-1-ylethyl)piperazine; N-(2-[(9Z)-octadec-9-enyloxy]-1-{[(9Z)-octadec-9-enyloxy]methyl}ethyl)-N-(3-pyrrolidin-1-ylpropyl)amine, 1-(2-[(9Z,12Z)-octadeca-9,12-dienyloxy]-1-{[(9Z,12Z)-octadeca-9,12-dienyloxy]methyl}ethyl)azetidine, 2-methyl-1-(2-[(9Z,12Z)-octadeca-9,12-dienyloxy]-1-{[(9Z,12Z)-octadeca-9,12-dienyloxy]methyl}ethyl)aziridine, 1-{3,4-bis[(9Z,12Z)-octadeca-9,12-dienyloxy]butyl}piperidine, 4-{3,4-bis[(9Z,12Z)-octadeca-9,12-dienyloxy]butyl}morpholine, N,N-diethyl-3,4-bis[(9Z,12Z)-octadeca-9,12-dienyloxy]butan-1-amine, N,N-dimethyl-3,4-bis[(9Z,12Z)-octadeca-9,12-dienyloxy]butan-1-amine, 1-{3,4-bis[(9Z,12Z)-octadeca-9,12-dienyloxy]butyl}-4-phenylpiperazine, 1-{3,4-bis[(9Z,12Z)-octadeca-9,12-dienyloxy]butyl}-4-methylpiperazine, N-(2-methoxyethyl)-N-methyl-3,4-bis[(9Z,12Z)-octadeca-9,12-dienyloxy]butan-1-amine, 1-{3,4-bis[(9Z,12Z)-octadeca-9,12-dienyloxy]butyl}-4-(2-methoxyphenyl)piperazine, N-{3,4-bis[(9Z,12Z)-octadeca-9,12-dienyloxy]butyl}-N,N′,N′-trimethylethane-1,2-diamine, N-{3,4-bis[(9Z,12Z)-octadeca-9,12-dienyloxy]butyl}-N-methyl-N-(2-pyridin-2-ylethyl)amine, N-benzyl-N-{3,4-bis[(9Z,12Z)-octadeca-9,12-dienyloxy]butyl}-N-methylamine, N-{3,4-bis[(9Z,12Z)-octadeca-9,12-dienyloxy]butyl}-N-(4-fluorobenzyl)-N-methylamine, 1-{3,4-bis[(9Z,12Z)-octadeca-9,12-dienyloxy]butyl}-4-(2-fluorophenyl)piperazine, N-benzyl-N-{3,4-bis[(9Z,12Z)-octadeca-9,12-dienyloxy]butyl}-N-ethylamine, N-{3,4-bis[(9Z,12Z)-octadeca-9,12-dienyloxy]butyl}-N-ethyl-N′,N′-dimethylethane-1,2-diamine, 1-{3,4-bis[(9Z,12Z)-octadeca-9,12-dienyloxy]butyl}-N,N-dimethylpiperidin-4-amine, 1-{3,4-bis[(9Z,12Z)-octadeca-9,12-dienyloxy]butyl}-N,N-dimethylpyrrolidin-3-amine, N,N-bis(2-methoxyethyl)-3,4-bis[(9Z,12Z)-octadeca-9,12-dienyloxy]butan-1-amine, 1-{3,4-bis[(9Z,12Z)-octadeca-9,12-dienyloxy]butyl}-4-methoxypiperidine, 1-{(3R)-3,4-bis[(9Z,12Z)-octadeca-9,12-dienyloxy]butyl}pyrrolidine, 1-{(3S)-3,4-bis[(9Z,12Z)-octadeca-9,12-dienyloxy]butyl}pyrrolidine, N-{(3R)-3,4-bis[(9Z,12Z)-octadeca-9,12-dienyloxy]butyl}-N,N-diethylamine, N-{(3S)-3,4-bis[(9Z,12Z)-octadeca-9,12-dienyloxy]butyl}-N,N-diethylamine, 1-{3,4-bis[(9Z,12Z)-octadeca-9,12-dienyloxy]butyl}pyrrolidine, N-(2-{3,4-bis[(9Z,12Z)-octadeca-9,12-dienyloxy]butoxy}ethyl)-N,N-diethylamine, 2-(2-{3,4-bis[(9Z,12Z)-octadeca-9,12-dienyloxy]butoxy}ethyl)-1-methylpyrrolidine, 1-(2-{3,4-bis[(9Z,12Z)-octadeca-9,12-dienyloxy]butoxy}ethyl)aziridine, 1-(2-{3,4-bis[(9Z,12Z)-octadeca-9,12-dienyloxy]butoxy}ethyl)-4-methylpiperazine, N-(2-{3,4-bis[(9Z,12Z)-octadeca-9,12-dienyloxy]butoxy}ethyl)-N,N-dimethylamine, 4-(diethylamino)-2-[(9Z,12Z)-octadeca-9,12-dienoyloxy]butyl (9Z,12Z)-octadeca-9,12-dienoate, 1-(2-{3,4-bis[(9Z,12Z)-octadeca-9,12-dienyloxy]butoxy}ethyl)pyrrolidine, N,N-diethyl-N-(2-{2-[(8Z,11Z)-heptadeca-8,11-dienyl]-2-[(9Z,12Z)-octadeca-9,12-dienyl]-1,3-dioxolan-4-yl}ethyl)amine, 1-{[(9Z)-octadec-9-enoyloxy]methyl}-3-pyrrolidin-1-ylpropyl (9Z)-octadec-9-enoate, 1-{3,4-bis[(9Z)-octadec-9-enyloxy]butyl}pyrrolidine, 1-{[(5Z,8Z,11Z,14Z)-icosa-5,8,11,14-tetraenoyloxy]methyl}-3-pyrrolidin-1-ylpropyl (5Z,8Z,11Z,14Z)-icosa-5,8,11,14-tetraenoate, (3S)-3,4-bis[(9Z,12Z)-octadeca-9,12-dienyloxy]butyl 3-pyrrolidin-1-ylpropylcarbamate, 1-[3,4-bis(octadecyloxy)butyl]pyrrolidine, 1-[3,4-bis(hexadecyloxy)butyl]pyrrolidine, 1-{3,4-bis[(9E)-hexadec-9-enyloxy]butyl}pyrrolidine, 1-{3,4-bis[(9E)-octadec-9-enyloxy]butyl}pyrrolidine, 1-{3,4-bis[(9E,12E)-octadeca-9,12-dienyloxy]butyl}pyrrolidine, 1-{3,4-bis[(9Z,12Z,15Z)-octadeca-9,12,15-trienyloxy]butyl}pyrrolidine, N¹-{(3S)-3,4-bis[(9Z,12Z)-octadeca-9,12-dienyloxy]butyl}-N³,N³-diethyl-beta-alaninamide, N-{3,4-bis[(9Z,12Z)-octadeca-9,12-dienyloxy]butyl}-N-[3-(1H-imidazol-1-yl)propyl]amine, N-{3,4-bis[(9Z,12Z)-octadeca-9,12-dienyloxy]butyl}-N,N′,N′-trimethylpropane-1,3-diamine, 1-(1-{3,4-bis[(9Z,12Z)-octadeca-9,12-dienyloxy]butyl}pyrrolidin-3-yl)-1H-imidazole, N-{3,4-bis[(9Z,12Z)-octadeca-9,12-dienyloxy]butyl}-N-(3-pyrrolidin-1-ylpropyl)amine, N-{3,4-bis[(9Z,12Z)-octadeca-9,12-dienyloxy]butyl}-N′,N′-dimethylpropane-1,3-diamine, 1-{3,4-bis[(9Z,12Z)-octadeca-9,12-dienyloxy]butyl}azetidine, 1-{3,4-bis[(9Z,12Z)-octadeca-9,12-dienyloxy]butyl}-2-methylpyrrolidine, 1-{3,4-bis[(9Z,12Z)-octadeca-9,12-dienyloxy]butyl}-2,5-dimethylpyrrolidine, are 1-(2,3-bis((9Z,12Z)-octadeca-9,12-dienyloxy)propyl)pyrrolidine, 1-(2,3-bis((9Z,12Z)-octadeca-9,12-dienyloxy)propyl)-1H-imidazole, 1-(2,3-bis((9Z,12Z)-octadeca-9,12-dienyloxy)propyl)-4-methylpiperazine, 1-(2,3-bis((9Z,12Z)-octadeca-9,12-dienyloxy)propyl)-4-methyl-1,4-diazepane, 1-(2,3-bis((9Z,12Z)-octadeca-9,12-dienyloxy)propyl)-4-phenylpiperazine, 1-(2,3-bis((9Z,12Z)-octadeca-9,12-dienyloxy)propyl)-4-pyridin-2-ylpiperazine, 1-(2,3-bis((9Z,12Z)-octadeca-9,12-dienyloxy)propyl)piperidine, 4-(2,3-bis((9Z,12Z)-octadeca-9,12-dienyloxy)propyl)morpholine, 1-((2R)-2,3-bis((9Z,12Z)-octadeca-9,12-dienyloxy)propyl)pyrrolidine, 1-((2S)-2,3-bis((9Z,12Z)-octadeca-9,12-dienyloxy)propyl)pyrrolidine, 1-(2,3-bis((9Z,12Z)-octadeca-9,12-dienyloxy)propyl)-4-ethylpiperazine, N-(2,3-bis((9Z,12Z)-octadeca-9,12-dienyloxy)propyl)-N-methyl-N-(3-(pyrrolidin-1-ylmethyl)benzyl)amine, N-(2-(4-(2,3-bis((9Z,12Z)-octadeca-9,12-dienyloxy)propyl)piperazin-1-yl)ethyl)-N,N-dimethylamine, 1-((2S)-2,3-bis((9Z,12Z)-octadeca-9,12-dienyloxy)propyl)-4-methylpiperazine, 1-((2R)-2,3-bis((9Z,12Z)-octadeca-9,12-dienyloxy)propyl)-4-methylpiperazine, 1-(2,3-bis((9Z,12Z)-octadeca-9,12-dienyloxy)propyl)-4-(2-pyrrolidin-1-ylethyl)piperazine, 2-(4-(2,3-bis((9Z,12Z)-octadeca-9,12-dienyloxy)propyl)piperazin-1-yl)pyrimidine, 1-(2,3-bis((9Z,12Z)-octadeca-9,12-dienyloxy)propyl)-N,N-diethylpyrrolidin-3-amine, 1-((9Z,12Z)-octadeca-9,12-dienyloxy)-3-pyrrolidin-1-ylpropan-2-ol, 2-[(9Z,12Z)-octadeca-9,12-dienoyloxy]-1-(pyrrolidin-1-ylmethyl)ethyl (9Z,12Z)-octadeca-9,12-dienoate, 2-[(9Z,12Z)-octadeca-9,12-dienyloxy]-1-(pyrrolidin-1-ylmethyl)ethyl (9Z,12Z)-octadeca-9,12-dienoate, 1-({2-[(8Z,11Z)-heptadeca-8,11-dienyl]-2-[(9Z,12Z)-octadeca-9,12-dienyl]-1,3-dioxolan-4-yl}methyl)pyrrolidine, 1-{2,3-bis[(5Z,8Z,11Z,14Z)-icosa-5,8,11,14-tetraenyloxy]propyl}pyrrolidine, 1-{3-[(5Z,8Z,11Z,14Z)-icosa-5,8,11,14-tetraenyloxy]-2-[(9Z,12Z)-octadeca-9,12-dienyloxy]propyl}pyrrolidine, 1-{2,3-bis[(9E,12E)-octadeca-9,12-dienyloxy]propyl}pyrrolidine, 1-{2-[(9E,12E)-octadeca-9,12-dienyloxy]-3-[(9Z,12Z)-octadeca-9,12-dienyloxy]propyl}pyrrolidine, 1-[2,3-bis(tetradecyloxy)propyl]pyrrolidine, 1-[2,3-bis(octadecyloxy)propyl]pyrrolidine, 1-{2,3-bis[(9Z)-octadec-9-enyloxy]propyl}pyrrolidine, 1-[2,3-bis(dodecyloxy)propyl]pyrrolidine, 1-{2,3-bis[(9Z,12Z)-octadeca-9,12-dienyloxy]propyl}pyrrolidin-3-ol, 1-{3-[(9Z,12Z)-hexadeca-9,12-dienyloxy]-2-[(9Z)-octadec-9-enyloxy]propyl}pyrrolidine, 1-{2,3-bis[(9Z,12Z)-octadeca-9,12-dienyloxy]propyl}-N,N-dimethylpyrrolidin-3-amine and 1-[3-[(9Z,12Z)-hexadeca-9,12-dienyloxy]-2-(tetradecyloxy)propyl]pyrrolidine, or a mixture thereof.

8. The CaBLES of claim 2, or the Lipid-Based Particle of claim 3, wherein the cationic lipid comprises about 2 to about 60 weight/weight percent of total lipid in the particle.

9. The compound of claim 1 which is 6-oxo-2-(tetradecanoyloxy)-8,11,14,17,20,23,26,29,32,35,38,41,44,47,50,53,56,59,62,65,68,71,74,77,80,83,86,89,92, 95,98,101,104,107,110,113,116,119,122,125,128,131,134,137,140,143-hexatetracontaoxa-5-azatetratetracontahect-1-yl myristate; N-[3,4-bis(tetradecyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide; N-[3,4-bis(hexadecyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide; N-[3,4-bis(octadecyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide; 3,7,11,15,19,23,27,31,35,39,43,47,51,55,59,63,67,71,75,79,83,87,91,95,99,103,107,111,115, 119,123,127,131,135,139,143,147,151,155,159,163,167,171,175,179,182-hexatetracontaoxatrioctacontahect-1-yl 3,4-bis(tetradecyloxy)butylcarbamate; 3,6,9,12,15,18,21,24,27,30,33,36,39,42,45,48,51,54,57,60,63,66,69,72,75,78,81,84,87,90, 93,96,99,102,105,108,111,114,117,120,123,126,129,132,135,138-hexatetracontaoxanonatriacontahect-1-yl 3,4-bis(hexadecyloxy)butylcarbamate; 3,6,9,12,15,18,21,24,27,30,33,36,39,42,45,48,51,54,57,60,63,66,69,72,75,78,81,84,87,90, 93,96,99,102,105,108,111,114,117,120,123,126,129,132,135,138-hexatetracontaoxanonatriacontahect-1-yl 3,4-bis(octadecyloxy)butylcarbamate; N-[3,4-bis(hexadecyloxy)butyl]-N′-3,6,9,12,15,18,21,24,27,30,33,36,39,42,45,48,51,54,57,60,63,66,69,72,75,78,81,84,87,90, 93,96,99,102,105,108,111,114,117,120,123,126,129,132,135,138-hexatetracontaoxanonatriacontahect-1-ylsuccinamide; 6-oxo-2-(tetradecanoyloxy)-7,10,13,16,19,22,25,28,31,34,37,40,43,46,49,52,55,58,61,64,67,70,73,76,79,82,85,88,91, 94,97,100,103,106,109,112,115,118,121,124,127,130,133,136,139,142,145-heptatetracontaoxa-5-azahexatetracontahect-1-yl myristate; 6-oxo-2-(palmitoyloxy)-7,10,13,16,19,22,25,28,31,34,37,40,43,46,49,52,55,58,61,64,67,70,73,76,79,82,85,88,91, 94,97,100,103,106,109,112,115,118,121,124,127,130,133,136,139,142,145-heptatetracontaoxa-5-azahexatetracontahect-1-yl palmitate; 3,6,9,12,15,18,21,24,27,30,33,36,39,42,45,48,51,54,57,60,63,66,69,72,75,78,81,84,87,90, 93,96,99,102,105,108,111,114,117,120,123,126,129,132,135,138-hexatetracontaoxanonatriacontahect-1-yl 4-{[3,4-bis(hexadecyloxy)butyl]amino}-4-oxobutanoate; 6-oxo-2-(palmitoyloxy)-8,11,14,17,20,23,26,29,32,35,38,41,44,47,50,53,56,59,62,65,68,71,74,77,80,83,86,89,92, 95,98,101,104,107,110,113,116,119,122,125,128,131,134,137,140,143-hexatetracontaoxa-5-azatetratetracontahect-1-yl palmitate; N-[4-(decyloxy)-3-(octadecyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide; N-[3,4-bis(decyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide; N-[3-(octadecyloxy)-4-(tetradecyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide; N-[4-(hexadecyloxy)-3-(octadecyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide; N-[3,4-bis(hexadecyloxy)butyl]-2,5,8,11,14,17,20,23,26,29,32,35,38,41,44,47,50,53,56,59,62,65,68-tricosaoxaheptacontan-70-amide; N-[3,4-bis(hexadecyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137,140,143,146,149,152,155, 158,161,164,167,170,173,176,179,182,185,188,191,194,197,200,203,206,209,212,215, 218,221,224,227,230,233,236,239,242,245,248,251,254,257,260,263,266,269,272,275,278, 281,284,287,290,293,296,299,302,305,308,311,314,317,320,323,326,329,332,335,338-113oxa340n-340-amide; or N-[3-(hexadecyloxy)-4-(octadecyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide.

10. The Lipid-Based Particle of claim 3, wherein the therapeutic agent is RNA, antisense oligonucleotide, a DNA, a plasmid, a ribozymal RNA (rRNA), a micro RNA (miRNA), transfer RNA (tRNA), a small inhibitory RNA (siRNA), small nuclear RNA (snRNA), an antigen, fragments thereof, a protein, a peptide, a small-molecule, or a mixture thereof.

11. The Lipid-Based Particle of claim 3, wherein said PEG lipid conjugate is about 0.1-20 weight/weight % of total lipid in particle, DSPC is about 1-30 weight/weight % of total lipid in particle, cholesterol is about 5-45 weight/weight % of total lipid in particle, and said cationic lipid is about 5-60 weight/weight % of total lipid in particle.

12. A pharmaceutical composition comprising a Lipid-Based Particle of claim 3 and a pharmaceutically acceptable carrier.

13. A pharmaceutical composition of claim 12, wherein said Lipid-Based Particle comprises cholesterol, DSPC, 1-(2,3-bis((9Z,12Z)-octadeca-9,12-dienyloxy)propyl)pyrrolidine, 2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide and one or more nucleic acids.

14. A pharmaceutical composition of claim 13, wherein said 2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide is about 1-25 weight/weight % of total lipid in particle, said DSPC is about 1-30 weight/weight % of total lipid in particle, said cholesterol is about 5-45 weight/weight % of total lipid in particle, and 1-(2,3-bis((9Z,12Z)-octadeca-9,12-dienyloxy)propyl)pyrrolidine is about 5-60 weight/weight % of total lipid in particle.

15. The Lipid-Based Particle of claim 3, wherein said non-cationic lipids are cholesterol and DSPC, said cationic lipid is 1-(2,3-bis((9Z,12Z)-octadeca-9,12-dienyloxy)propyl)pyrrolidine, said PEG-lipid conjugate is 2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide, and said therapeutic agent is siRNA.

16. The Lipid-Based Particle of claim 15, wherein said 2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide is about 1-25 weight/weight % of total lipid in particle, said DSPC is about 1-30 weight/weight % of total lipid in particle, said cholesterol is about 5-45 weight/weight % of total lipid in particle, and said 1-(2,3-bis((9Z,12Z)-octadeca-9,12-dienyloxy)propyl)pyrrolidine is about 5-60 weight/weight % of total lipid in particle.

17. A pharmaceutical composition of claim 12, wherein said Lipid-Based Particle comprises cholesterol, DSPC, 1-(2,3-bis((9Z,12Z)-octadeca-9,12-dienyloxy)propyl)pyrrolidine, N-[3,4-bis(tetradecyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanon atriacontahectan-139-amide and one or more nucleic acids.

18. A pharmaceutical composition of claim 17, wherein said N-[3,4-bis(tetradecyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanon atriacontahectan-139-amide is about 1-25 weight/weight % of total lipid in particle, said DSPC is about 1-30 weight/weight % of total lipid in particle, said cholesterol is about 5-45 weight/weight % of total lipid in particle, and 1-(2,3-bis((9Z,12Z)-octadeca-9,12-dienyloxy)propyl)pyrrolidine is about 5-60 weight/weight % of total lipid in particle.

19. The Lipid-Based Particle of claim 3, wherein said non-cationic lipids are cholesterol and DSPC, said cationic lipid is 1-(2,3-bis((9Z,12Z)-octadeca-9,12-dienyloxy)propyl)pyrrolidine, said PEG-lipid conjugate is N-[3,4-bis(tetradecyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanon atriacontahectan-139-amide, and said therapeutic agent is siRNA.

20. The Lipid-Based Particle of claim 19, wherein said N-[3,4-bis(tetradecyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanon atriacontahectan-139-amide is about 1-25 weight/weight % of total lipid in particle, said DSPC is about 1-30 weight/weight % of total lipid in particle, said cholesterol is about 5-45 weight/weight % of total lipid in particle, and said 1-(2,3-bis((9Z,12Z)-octadeca-9,12-dienyloxy)propyl)pyrrolidine is about 5-60 weight/weight % of total lipid in particle.

21. A pharmaceutical composition of claim 12, wherein said Lipid-Based Particle comprises cholesterol, DSPC, 1-(2,3-bis((9Z,12Z)-octadeca-9,12-dienyloxy)propyl)pyrrolidine, 2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide, 1,2-distearoyl-sn-glycerol-methoxypolyethyleneglycol-2000, and one or more nucleic acids.

22. A pharmaceutical composition of claim 13, wherein said 2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide and 1,2-distearoyl-sn-glycerol-methoxypolyethyleneglycol-2000 are about 1-25 weight/weight % of total lipid in particle, said DSPC is about 1-30 weight/weight % of total lipid in particle, said cholesterol is about 5-45 weight/weight % of total lipid in particle, and 1-(2,3-bis((9Z,12Z)-octadeca-9,12-dienyloxy)propyl)pyrrolidine is about 5-60 weight/weight % of total lipid in particle.

23. The Lipid-Based Particle of claim 3, wherein said non-cationic lipids are cholesterol and DSPC, said cationic lipid is 1-(2,3-bis((9Z,12Z)-octadeca-9,12-dienyloxy)propyl)pyrrolidine, said PEG-lipid conjugates are 2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide and 1,2-distearoyl-sn-glycerol-methoxypolyethyleneglycol-2000, and said therapeutic agent is siRNA.

24. The Lipid-Based Particle of claim 23, wherein said 2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide and 1,2-distearoyl-sn-glycerol-methoxypolyethyleneglycol-2000 are about 1-25 weight/weight % of total lipid in particle, said DSPC is about 1-30 weight/weight % of total lipid in particle, said cholesterol is about 5-45 weight/weight % of total lipid in particle, and said 1-(2,3-bis((9Z,12Z)-octadeca-9,12-dienyloxy)propyl)pyrrolidine is about 5-60 weight/weight % of total lipid in particle.

25. A pharmaceutical composition of claim 12, wherein said Lipid-Based Particle comprises cholesterol, DSPC, 1-(2,3-bis((9Z,12Z)-octadeca-9,12-dienyloxy)propyl)pyrrolidine, 2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide, N-(carbonyl-methoxypolyethyleneglycol-2000)-1,2-distearoyl-sn-glycero-3-phosphoethanolamine, and one or more nucleic acids.

26. A pharmaceutical composition of claim 25, wherein said 2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide and N-(carbonyl-methoxypolyethyleneglycol-2000)-1,2-distearoyl-sn-glycero-3-phosphoethanolamine are about 1-25 weight/weight % of total lipid in particle, said DSPC is about 1-30 weight/weight % of total lipid in particle, said cholesterol is about 5-45 weight/weight % of total lipid in particle, and 1-(2,3-bis((9Z,12Z)-octadeca-9,12-dienyloxy)propyl)pyrrolidine is about 5-60 weight/weight % of total lipid in particle.

27. The Lipid-Based Particle of claim 3, wherein said non-cationic lipids are cholesterol and DSPC, said cationic lipid is 1-(2,3-bis((9Z,12Z)-octadeca-9,12-dienyloxy)propyl)pyrrolidine, said PEG-lipid conjugates are 2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide and N-(carbonyl-methoxypolyethyleneglycol-2000)-1,2-distearoyl-sn-glycero-3-phosphoethanolamine, and said therapeutic agent is siRNA.

28. The Lipid-Based Particle of claim 27, wherein said 2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide and N-(carbonyl-methoxypolyethyleneglycol-2000)-1,2-distearoyl-sn-glycero-3-phosphoethanolamine are about 1-25 weight/weight % of total lipid in particle, said DSPC is about 1-30 weight/weight % of total lipid in particle, said cholesterol is about 5-45 weight/weight % of total lipid in particle, and said 1-(2,3-bis((9Z,12Z)-octadeca-9,12-dienyloxy)propyl)pyrrolidine is about 5-60 weight/weight % of total lipid in particle.

29. A pharmaceutical composition of claim 12, wherein said Lipid-Based Particle comprises cholesterol, DSPC, 1-{3,4-bis[(9Z,12Z)-octadeca-9,12-dienyloxy]butyl}pyrrolidine, 2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide and one or more nucleic acids.

30. A pharmaceutical composition of claim 29, wherein said 2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide is about 1-25 weight/weight % of total lipid in particle, said DSPC is about 1-30 weight/weight % of total lipid in particle, said cholesterol is about 5-45 weight/weight % of total lipid in particle, and 1-{3,4-bis[(9Z,12Z)-octadeca-9,12-dienyloxy]butyl}pyrrolidine is about 5-60 weight/weight % of total lipid in particle.

31. The Lipid-Based Particle of claim 3, wherein said non-cationic lipids are cholesterol and DSPC, said cationic lipid is 1-{3,4-bis[(9Z,12Z)-octadeca-9,12-dienyloxy]butyl}pyrrolidine, said PEG-lipid conjugate is 2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide, and said therapeutic agent is siRNA.

32. The Lipid-Based Particle of claim 31, wherein said 2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide is about 1-25 weight/weight % of total lipid in particle, said DSPC is about 1-30 weight/weight % of total lipid in particle, said cholesterol is about 5-45 weight/weight % of total lipid in particle, and said 1-{3,4-bis[(9Z,12Z)-octadeca-9,12-dienyloxy]butyl}pyrrolidine is about 5-60 weight/weight % of total lipid in particle.

33. A pharmaceutical composition of claim 12, wherein said Lipid-Based Particle comprises cholesterol, DSPC, 1-{3,4-bis[(9Z,12Z)-octadeca-9,12-dienyloxy]butyl}pyrrolidine, N-[3,4-bis(octadecyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide and one or more nucleic acids.

34. A pharmaceutical composition of claim 33, wherein said N-[3,4-bis(octadecyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide is about 1-25 weight/weight % of total lipid in particle, said DSPC is about 1-30 weight/weight % of total lipid in particle, said cholesterol is about 5-45 weight/weight % of total lipid in particle, and 1-{3,4-bis[(9Z,12Z)-octadeca-9,12-dienyloxy]butyl}pyrrolidine is about 5-60 weight/weight % of total lipid in particle.

35. The Lipid-Based Particle of claim 3, wherein said non-cationic lipids are cholesterol and DSPC, said cationic lipid is 1-{3,4-bis[(9Z,12Z)-octadeca-9,12-dienyloxy]butyl}pyrrolidine, said PEG-lipid conjugate is N-[3,4-bis(octadecyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide, and said therapeutic agent is siRNA.

36. The Lipid-Based Particle of claim 35, wherein said N-[3,4-bis(octadecyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide is about 1-25 weight/weight % of total lipid in particle, said DSPC is about 1-30 weight/weight % of total lipid in particle, said cholesterol is about 5-45 weight/weight % of total lipid in particle, and said 1-{3,4-bis[(9Z,12Z)-octadeca-9,12-dienyloxy]butyl}pyrrolidine is about 5-60 weight/weight % of total lipid in particle.

37. A pharmaceutical composition of claim 12, wherein said Lipid-Based Particle comprises cholesterol, DSPC, 1-(2,3-bis((9Z,12Z)-octadeca-9,12-dienyloxy)propyl)pyrrolidine, 6-oxo-2-(palmitoyloxy)-8,11,14,17,20,23,26,29,32,35,38,41,44,47,50,53,56,59,62,65,68,71,74,77,80,83,86,89,92, 95,98,101,104,107,110,113,116,119,122,125,128,131,134,137,140,143-hexatetracontaoxa-5-azatetratetracontahect-1-yl palmitate and one or more nucleic acids.

38. A pharmaceutical composition of claim 37, wherein said 6-oxo-2-(palmitoyloxy)-8,11,14,17,20,23,26,29,32,35,38,41,44,47,50,53,56,59,62,65,68,71,74,77,80,83,86,89,92, 95,98,101,104,107,110,113,116,119,122,125,128,131,134,137,140,143-hexatetracontaoxa-5-azatetratetracontahect-1-yl palmitate is about 1-25 weight/weight % of total lipid in particle, said DSPC is about 1-30 weight/weight % of total lipid in particle, said cholesterol is about 5-45 weight/weight % of total lipid in particle, and 1-(2,3-bis((9Z,12Z)-octadeca-9,12-dienyloxy)propyl)pyrrolidine is about 5-60 weight/weight % of total lipid in particle.

39. The Lipid-Based Particle of claim 3, wherein said non-cationic lipids are cholesterol and DSPC, said cationic lipid is 1-(2,3-bis((9Z,12Z)-octadeca-9,12-dienyloxy)propyl)pyrrolidine, said PEG-lipid conjugate is 6-oxo-2-(palmitoyloxy)-8,11,14,17,20,23,26,29,32,35,38,41,44,47,50,53,56,59,62,65,68,71,74,77,80,83,86,89,92, 95,98,101,104,107,110,113,116,119,122,125,128,131,134,137,140,143-hexatetracontaoxa-5-azatetratetracontahect-1-yl palmitate, and said therapeutic agent is siRNA.

40. The Lipid-Based Particle of claim 39, wherein said 6-oxo-2-(palmitoyloxy)-8,11,14,17,20,23,26,29,32,35,38,41,44,47,50,53,56,59,62,65,68,71,74,77,80,83,86,89,92, 95,98,101,104,107,110,113,116,119,122,125,128,131,134,137,140,143-hexatetracontaoxa-5-azatetratetracontahect-1-yl palmitate is about 1-25 weight/weight % of total lipid in particle, said DSPC is about 1-30 weight/weight % of total lipid in particle, said cholesterol is about 5-45 weight/weight % of total lipid in particle, and said 1-(2,3-bis((9Z,12Z)-octadeca-9,12-dienyloxy)propyl)pyrrolidine is about 5-60 weight/weight % of total lipid in particle.

41. A pharmaceutical composition of claim 12, wherein said Lipid-Based Particle comprises cholesterol, DSPC, 1-(2,3-bis((9Z,12Z)-octadeca-9,12-dienyloxy)propyl)pyrrolidine, N-[3,4-bis(hexadecyloxy)butyl]-N′-3,6,9,12,15,18,21,24,27,30,33,36,39,42,45,48,51,54,57,60,63,66,69,72,75,78,81,84,87,90, 93,96,99,102,105,108,111,114,117,120,123,126,129,132,135,138-hexatetracontaoxanonatriacontahect-1-ylsuccinamide and one or more nucleic acids.

42. A pharmaceutical composition of claim 41, wherein said N-[3,4-bis(hexadecyloxy)butyl]-N′-3,6,9,12,15,18,21,24,27,30,33,36,39,42,45,48,51,54,57,60,63,66,69,72,75,78,81,84,87,90, 93,96,99,102,105,108,111,114,117,120,123,126,129,132,135,138-hexatetracontaoxanonatriacontahect-1-ylsuccinamide is about 1-25 weight/weight % of total lipid in particle, said DSPC is about 1-30 weight/weight % of total lipid in particle, said cholesterol is about 5-45 weight/weight % of total lipid in particle, and 1-(2,3-bis((9Z,12Z)-octadeca-9,12-dienyloxy)propyl)pyrrolidine is about 5-60 weight/weight % of total lipid in particle.

43. The Lipid-Based Particle of claim 3, wherein said non-cationic lipids are cholesterol and DSPC, said cationic lipid is 1-(2,3-bis((9Z,12Z)-octadeca-9,12-dienyloxy)propyl)pyrrolidine, said PEG-lipid conjugate is N-[3,4-bis(hexadecyloxy)butyl]-N′-3,6,9,12,15,18,21,24,27,30,33,36,39,42,45,48,51,54,57,60,63,66,69,72,75,78,81,84,87,90, 93,96,99,102,105,108,111,114,117,120,123,126,129,132,135,138-hexatetracontaoxanonatriacontahect-1-ylsuccinamide, and said therapeutic agent is siRNA.

44. The Lipid-Based Particle of claim 43, wherein said N-[3,4-bis(hexadecyloxy)butyl]-N′-3,6,9,12,15,18,21,24,27,30,33,36,39,42,45,48,51,54,57,60,63,66,69,72,75,78,81,84,87,90, 93,96,99,102,105,108,111,114,117,120,123,126,129,132,135,138-hexatetracontaoxanonatriacontahect-1-ylsuccinamide is about 1-25 weight/weight % of total lipid in particle, said DSPC is about 1-30 weight/weight % of total lipid in particle, said cholesterol is about 5-45 weight/weight % of total lipid in particle, and said 1-(2,3-bis((9Z,12Z)-octadeca-9,12-dienyloxy)propyl)pyrrolidine is about 5-60 weight/weight % of total lipid in particle.

45. A pharmaceutical composition of claim 12, wherein said Lipid-Based Particle comprises cholesterol, DSPC, 1-(2,3-bis((9Z,12Z)-octadeca-9,12-dienyloxy)propyl)pyrrolidine, 3,6,9,12,15,18,21,24,27,30,33,36,39,42,45,48,51,54,57,60,63,66,69,72,75,78,81,84,87,90, 93,96,99,102,105,108,111,114,117,120,123,126,129,132,135,138-hexatetracontaoxanonatriacontahect-1-yl 4-{[3,4-bis(hexadecyloxy)butyl]amino}-4-oxobutanoate and one or more nucleic acids.

46. A pharmaceutical composition of claim 45, wherein said 3,6,9,12,15,18,21,24,27,30,33,36,39,42,45,48,51,54,57,60,63,66,69,72,75,78,81,84,87,90, 93,96,99,102,105,108,111,114,117,120,123,126,129,132,135,138-hexatetracontaoxanonatriacontahect-1-yl 4-{[3,4-bis(hexadecyloxy)butyl]amino}-4-oxobutanoate is about 1-25 weight/weight % of total lipid in particle, said DSPC is about 1-30 weight/weight % of total lipid in particle, said cholesterol is about 5-45 weight/weight % of total lipid in particle, and 1-(2,3-bis((9Z,12Z)-octadeca-9,12-dienyloxy)propyl)pyrrolidine is about 5-60 weight/weight % of total lipid in particle.

47. The Lipid-Based Particle of claim 3, wherein said non-cationic lipids are cholesterol and DSPC, said cationic lipid is 1-(2,3-bis((9Z,12Z)-octadeca-9,12-dienyloxy)propyl)pyrrolidine, said PEG-lipid conjugate is 3,6,9,12,15,18,21,24,27,30,33,36,39,42,45,48,51,54,57,60,63,66,69,72,75,78,81,84,87,90, 93,96,99,102,105,108,111,114,117,120,123,126,129,132,135,138-hexatetracontaoxanonatriacontahect-1-yl 4-{[3,4-bis(hexadecyloxy)butyl]amino}-4-oxobutanoate, and said therapeutic agent is siRNA.

48. The Lipid-Based Particle of claim 47, wherein said 3,6,9,12,15,18,21,24,27,30,33,36,39,42,45,48,51,54,57,60,63,66,69,72,75,78,81,84,87,90, 93,96,99,102,105,108,111,114,117,120,123,126,129,132,135,138-hexatetracontaoxanonatriacontahect-1-yl 4-{[3,4-bis(hexadecyloxy)butyl]amino}-4-oxobutanoate is about 1-25 weight/weight % of total lipid in particle, said DSPC is about 1-30 weight/weight % of total lipid in particle, said cholesterol is about 5-45 weight/weight % of total lipid in particle, and said 1-(2,3-bis((9Z,12Z)-octadeca-9,12-dienyloxy)propyl)pyrrolidine is about 5-60 weight/weight % of total lipid in particle.

49. A pharmaceutical composition of claim 12, wherein said Lipid-Based Particle comprises cholesterol, DSPC, 1-(2,3-bis((9Z,12Z)-octadeca-9,12-dienyloxy)propyl)pyrrolidine, N-[4-(decyloxy)-3-(octadecyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide and one or more nucleic acids.

50. A pharmaceutical composition of claim 49, wherein said N-[4-(decyloxy)-3-(octadecyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide is about 1-25 weight/weight % of total lipid in particle, said DSPC is about 1-30 weight/weight % of total lipid in particle, said cholesterol is about 5-45 weight/weight % of total lipid in particle, and 1-(2,3-bis((9Z,12Z)-octadeca-9,12-dienyloxy)propyl)pyrrolidine is about 5-60 weight/weight % of total lipid in particle.

51. The Lipid-Based Particle of claim 3, wherein said non-cationic lipids are cholesterol and DSPC, said cationic lipid is 1-(2,3-bis((9Z,12Z)-octadeca-9,12-dienyloxy)propyl)pyrrolidine, said PEG-lipid conjugate is N-[4-(decyloxy)-3-(octadecyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide, and said therapeutic agent is siRNA.

52. The Lipid-Based Particle of claim 51, wherein said N-[4-(decyloxy)-3-(octadecyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide is about 1-25 weight/weight % of total lipid in particle, said DSPC is about 1-30 weight/weight % of total lipid in particle, said cholesterol is about 5-45 weight/weight % of total lipid in particle, and said 1-(2,3-bis((9Z,12Z)-octadeca-9,12-dienyloxy)propyl)pyrrolidine is about 5-60 weight/weight % of total lipid in particle.

53. A pharmaceutical composition of claim 12, wherein said Lipid-Based Particle comprises cholesterol, DSPC, 1-(2,3-bis((9Z,12Z)-octadeca-9,12-dienyloxy)propyl)pyrrolidine, N-[3-(octadecyloxy)-4-(tetradecyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide and one or more nucleic acids.

54. A pharmaceutical composition of claim 53, wherein said N-[3-(octadecyloxy)-4-(tetradecyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide is about 1-25 weight/weight % of total lipid in particle, said DSPC is about 1-30 weight/weight % of total lipid in particle, said cholesterol is about 5-45 weight/weight % of total lipid in particle, and 1-(2,3-bis((9Z,12Z)-octadeca-9,12-dienyloxy)propyl)pyrrolidine is about 5-60 weight/weight % of total lipid in particle.

55. The Lipid-Based Particle of claim 3, wherein said non-cationic lipids are cholesterol and DSPC, said cationic lipid is 1-(2,3-bis((9Z,12Z)-octadeca-9,12-dienyloxy)propyl)pyrrolidine, said PEG-lipid conjugate is N-[3-(octadecyloxy)-4-(tetradecyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide, and said therapeutic agent is siRNA.

56. The Lipid-Based Particle of claim 55, wherein said N-[3-(octadecyloxy)-4-(tetradecyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide is about 1-25 weight/weight % of total lipid in particle, said DSPC is about 1-30 weight/weight % of total lipid in particle, said cholesterol is about 5-45 weight/weight % of total lipid in particle, and said 1-(2,3-bis((9Z,12Z)-octadeca-9,12-dienyloxy)propyl)pyrrolidine is about 5-60 weight/weight % of total lipid in particle.

57. A pharmaceutical composition of claim 12, wherein said Lipid-Based Particle comprises cholesterol, DSPC, 1-(2,3-bis((9Z,12Z)-octadeca-9,12-dienyloxy)propyl)pyrrolidine, N-[3,4-bis(hexadecyloxy)butyl]-2,5,8,11,14,17,20,23,26,29,32,35,38,41,44,47,50,53,56,59,62,65,68-tricosaoxaheptacontan-70-amide and one or more nucleic acids.

58. A pharmaceutical composition of claim 57, wherein said N-[3,4-bis(hexadecyloxy)butyl]-2,5,8,11,14,17,20,23,26,29,32,35,38,41,44,47,50,53,56,59,62,65,68-tricosaoxaheptacontan-70-amide is about 1-25 weight/weight % of total lipid in particle, said DSPC is about 1-30 weight/weight % of total lipid in particle, said cholesterol is about 5-45 weight/weight % of total lipid in particle, and 1-(2,3-bis((9Z,12Z)-octadeca-9,12-dienyloxy)propyl)pyrrolidine is about 5-60 weight/weight % of total lipid in particle.

59. The Lipid-Based Particle of claim 3, wherein said non-cationic lipids are cholesterol and DSPC, said cationic lipid is 1-(2,3-bis((9Z,12Z)-octadeca-9,12-dienyloxy)propyl)pyrrolidine, said PEG-lipid conjugate is N-[3,4-bis(hexadecyloxy)butyl]-2,5,8,11,14,17,20,23,26,29,32,35,38,41,44,47,50,53,56,59,62,65,68-tricosaoxaheptacontan-70-amide, and said therapeutic agent is siRNA.

60. The Lipid-Based Particle of claim 59, wherein said N-[3,4-bis(hexadecyloxy)butyl]-2,5,8,11,14,17,20,23,26,29,32,35,38,41,44,47,50,53,56,59,62,65,68-tricosaoxaheptacontan-70-amide is about 1-25 weight/weight % of total lipid in particle, said DSPC is about 1-30 weight/weight % of total lipid in particle, said cholesterol is about 5-45 weight/weight % of total lipid in particle, and said 1-(2,3-bis((9Z,12Z)-octadeca-9,12-dienyloxy)propyl)pyrrolidine is about 5-60 weight/weight % of total lipid in particle.

61. A pharmaceutical composition of claim 12, wherein said Lipid-Based Particle comprises cholesterol, DSPC, 1-(2,3-bis((9Z,12Z)-octadeca-9,12-dienyloxy)propyl)pyrrolidine, N-[3,4-bis(hexadecyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137,140,143,146,149,152,155, 158,161,164,167,170,173,176,179,182,185,188,191,194,197,200,203,206,209,212,215, 218,221,224,227,230,233,236,239,242,245,248,251,254,257,260,263,266,269,272,275,278, 281,284,287,290,293,296,299,302,305,308,311,314,317,320,323,326,329,332,335,338-113oxa340n-340-amide and one or more nucleic acids.

62. A pharmaceutical composition of claim 61, wherein said N-[3,4-bis(hexadecyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137,140,143,146,149,152,155, 158,161,164,167,170,173,176,179,182,185,188,191,194,197,200,203,206,209,212,215, 218,221,224,227,230,233,236,239,242,245,248,251,254,257,260,263,266,269,272,275,278, 281,284,287,290,293,296,299,302,305,308,311,314,317,320,323,326,329,332,335,338-113oxa340n-340-amide is about 1-25 weight/weight % of total lipid in particle, said DSPC is about 1-30 weight/weight % of total lipid in particle, said cholesterol is about 5-45 weight/weight % of total lipid in particle, and 1-(2,3-bis((9Z,12Z)-octadeca-9,12-dienyloxy)propyl)pyrrolidine is about 5-60 weight/weight % of total lipid in particle.

63. The Lipid-Based Particle of claim 3, wherein said non-cationic lipids are cholesterol and DSPC, said cationic lipid is 1-(2,3-bis((9Z,12Z)-octadeca-9,12-dienyloxy)propyl)pyrrolidine, said PEG-lipid conjugate is N-[3,4-bis(hexadecyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137,140,143,146,149,152,155, 158,161,164,167,170,173,176,179,182,185,188,191,194,197,200,203,206,209,212,215, 218,221,224,227,230,233,236,239,242,245,248,251,254,257,260,263,266,269,272,275,278, 281,284,287,290,293,296,299,302,305,308,311,314,317,320,323,326,329,332,335,338-113oxa340n-340-amide, and said therapeutic agent is siRNA.

64. The Lipid-Based Particle of claim 63, wherein said N-[3,4-bis(hexadecyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137,140,143,146,149,152,155, 158,161,164,167,170,173,176,179,182,185,188,191,194,197,200,203,206,209,212,215, 218,221,224,227,230,233,236,239,242,245,248,251,254,257,260,263,266,269,272,275,278, 281,284,287,290,293,296,299,302,305,308,311,314,317,320,323,326,329,332,335,338-113oxa340n-340-amide is about 1-25 weight/weight % of total lipid in particle, said DSPC is about 1-30 weight/weight % of total lipid in particle, said cholesterol is about 5-45 weight/weight % of total lipid in particle, and said 1-(2,3-bis((9Z,12Z)-octadeca-9,12-dienyloxy)propyl)pyrrolidine is about 5-60 weight/weight % of total lipid in particle.

65. A pharmaceutical composition of claim 12, wherein said Lipid-Based Particle comprises cholesterol, DSPC, 1-(2,3-bis((9Z,12Z)-octadeca-9,12-dienyloxy)propyl)pyrrolidine, N-[3-(hexadecyloxy)-4-(octadecyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide and one or more nucleic acids.

66. A pharmaceutical composition of claim 65, wherein said N-[3-(hexadecyloxy)-4-(octadecyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide is about 1-25 weight/weight % of total lipid in particle, said DSPC is about 1-30 weight/weight % of total lipid in particle, said cholesterol is about 5-45 weight/weight % of total lipid in particle, and 1-(2,3-bis((9Z,12Z)-octadeca-9,12-dienyloxy)propyl)pyrrolidine is about 5-60 weight/weight % of total lipid in particle.

67. The Lipid-Based Particle of claim 3, wherein said non-cationic lipids are cholesterol and DSPC, said cationic lipid is 1-(2,3-bis((9Z,12Z)-octadeca-9,12-dienyloxy)propyl)pyrrolidine, said PEG-lipid conjugate is N-[3-(hexadecyloxy)-4-(octadecyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide, and said therapeutic agent is siRNA.

68. The Lipid-Based Particle of claim 67, wherein said N-[3-(hexadecyloxy)-4-(octadecyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide is about 1-25 weight/weight % of total lipid in particle, said DSPC is about 1-30 weight/weight % of total lipid in particle, said cholesterol is about 5-45 weight/weight % of total lipid in particle, and said 1-(2,3-bis((9Z,12Z)-octadeca-9,12-dienyloxy)propyl)pyrrolidine is about 5-60 weight/weight % of total lipid in particle.

69. The Lipid-Based Particle of claim 3, wherein said non-cationic lipids are cholesterol and DSPC, said cationic lipid is 1-(2,3-bis((9Z,12Z)-octadeca-9,12-dienyloxy)propyl)pyrrolidine, said PEG-lipid conjugate is 6-oxo-2-(palmitoyloxy)-8,11,14,17,20,23,26,29,32,35,38,41,44,47,50,53,56,59,62,65,68,71,74,77,80,83,86,89,92, 95,98,101,104,107,110,113,116,119,122,125,128,131,134,137,140,143-hexatetracontaoxa-5-azatetratetracontahect-1-yl palmitate, and said therapeutic agent is siRNA.

70. The Lipid-Based Particle of claim 3, wherein said non-cationic lipids are cholesterol and DSPC, said cationic lipid is 1-(2,3-bis((9Z,12Z)-octadeca-9,12-dienyloxy)propyl)pyrrolidine, said PEG-lipid conjugate is N-[3,4-bis(hexadecyloxy)butyl]-N′-3,6,9,12,15,18,21,24,27,30,33,36,39,42,45,48,51,54,57,60,63,66,69,72,75,78,81,84,87,90, 93,96,99,102,105,108,111,114,117,120,123,126,129,132,135,138-hexatetracontaoxanonatriacontahect-1-ylsuccinamide, and said therapeutic agent is siRNA.

71. The Lipid-Based Particle of claim 3, wherein said non-cationic lipids are cholesterol and DSPC, said cationic lipid is 1-(2,3-bis((9Z,12Z)-octadeca-9,12-dienyloxy)propyl)pyrrolidine, said PEG-lipid conjugate is N-[3-(octadecyloxy)-4-(tetradecyloxy)butyl]-2,5,8,11,14,17,20,23,26,29,32,35,38,41,44,47,50,53,56,59,62,65,68,71,74,77,80,83,86,89, 92,95,98,101,104,107,110,113,116,119,122,125,128,131,134,137-hexatetracontaoxanonatriacontahectan-139-amide, and said therapeutic agent is siRNA.

72. The Lipid-Based Particle of claim 3, wherein said non-cationic lipids are cholesterol and DSPC, said cationic lipid is 1-(2,3-bis((9Z,12Z)-octadeca-9,12-dienyloxy)propyl)pyrrolidine, said PEG-lipid conjugate is N-[3,4-bis(hexadecyloxy)butyl]-2,5,8,11,14,17,20,23,26,29,32,35,38,41,44,47,50,53,56,59,62,65,68-tricosaoxaheptacontan-70-amide, and said therapeutic agent is siRNA.

73. A method of making the Lipid-Based Particle of claim 3, comprising:
(a) mixing the cationic lipid(s), the non-cationic lipid(s) and the PEG-lipid conjugate(s);
(b) adding the mixture of step (a) to one or more therapeutic agents; and
(c) separating and purifying resulting suspension of step (b).

74. The method of claim 69, wherein said mixture of step (a) and one or more said therapeutic agents are warmed to about 60° C. prior to the addition of said mixture of step (a) to one or more therapeutic agents via needle injection.

75. The CaBLES of claim 2 which effectively encapsulate therapeutic agents, with efficiencies from about 50-100%.

76. The CaBLES of claim 2 which effectively encapsulate therapeutic agents, with efficiencies from about 80-100%.

77. The Lipid-Based Particle of claim 3, wherein the ratio of one or more (PEG)-lipid conjugates, one or more non-cationic lipids, and one or more cationic lipids of claim 1, to one or more therapeutic agents is between about 50:1 to about 5:1.

78. The Lipid-Based Particle of claim 3, wherein the ratio of one or more (PEG)-lipid conjugates, one or more non-cationic lipids, and one or more cationic lipids of claim 1, to one or more therapeutic agents is between about 30:1 to about 10:1.


SEQUENCE LISTING (1 sequence)

SEQ ID NO: 1 - RNA, 18 nt, Artificial Sequence
  siRNA sequence
ugguuuacauguugugug